(19) United States
(12) Patent Application Publication
(10) Pub. No.: US 2023/0192745 A1
(43) Pub. Date: Jun. 22, 2023

(54) NOVEL INTERMEDIATES FOR THE PREPARATION OF GBS POLYSACCHARIDE ANTIGENS

(71) Applicant: GLAXOSMITHKLINE BIOLOGICALS SA, Rixensart (BE)

(72) Inventors: Roberto ADAMO, Siena (IT); Linda DEL BINO, Siena (IT)

(73) Assignee: GLAXOSMITHKLINE BIOLOGICALS SA, Rixensart (BE)

(21) Appl. No.: 16/620,900

(22) PCT Filed: Jul. 12, 2018
(86) PCT No.: PCT/IB2018/055158

§ 371 Date: Dec. 10, 2019

(30) Foreign Application Priority Data
Jul. 13, 2017 (GB) 1711274.9

(51) Int. Cl. C07H 1/06; C07H 15/04; A61K 47/64

(52) U.S. Cl. CPC C07H 1/06; C07H 15/04; A61K 47/6415

(57) ABSTRACT

The present invention generally refers to novel intermediate polysaccharide units, useful for the preparation of polysaccharide antigen of GBS Ia, Ib and III; the invention also refers to a process for their preparation and their use as intermediate for the preparation of conjugated derivatives useful in vaccines.

### CROSS-REFERENCE TO RELATED APPLICATIONS

This application is filed pursuant to 35 U.S.C. § 371 as a United States National Phase Application of International Application No. PCT/IB2018/055158 filed Jul. 12, 2018 which claims priority from GB 1711274.9 filed Jul. 13, 2017.

### FIELD OF THE INVENTION

The present invention generally refers to novel intermediate polysaccharide units, useful for the preparation of polysaccharide antigen of GBS Ia, Ib and III; the invention also refers to a process for their preparation and their use as intermediate for the preparation of conjugated derivatives useful in vaccines.

### BACKGROUND OF THE INVENTION

Despite the enormous structural variability of carbohydrates, some motifs are recurrently expressed both by prokaryotic and eukaryotic cells: one example is the disaccharide GlcNAc-β-(1→3) Gal, which is present in several bacterial carbohydrates, including the capsular polysaccharides (PSs) of Group B Streptococcus (GBS) type Ia, Ib and III, and Streptococcus pneumonia type 14 (Pn14), as well as Neisseriae meningitidis lipooligosaccharide (LOS). Particularly, in GBS PS Ia and Ib, the GlcNAc-β-(1→3) Gal disaccharide is further β-(1→4) substituted at position 4 of Gal with a Glc residue.

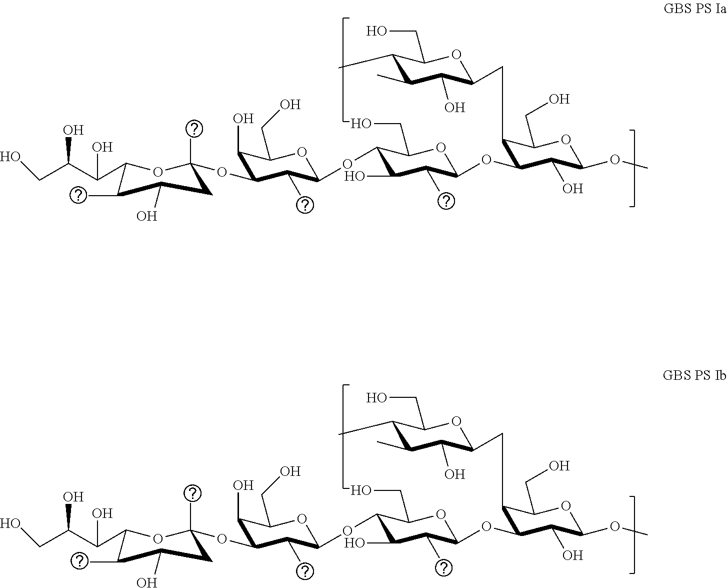

Synthesis of the GlcNAc-β-(1→3) Gal disaccharide generally requires a Gal acceptor protected on 4-OH. For instance, Craft et al. in Synthesis of lacto-N-tetraose. Carbohydr Res 2017, 440-441, 43-50. recently observed that a 4-O-acetyl group was needed in the Gal acceptor to achieve glycosylation at position 3 with an N-Trichloroethoxycarbamoyl protected glucosamine (GlcN) donor. This finding was in line with previous reports (see e.g. Pozsgay, V.; Gaudino, J.; Paulson, J. C.; Jennings, H. J., Chemo-enzymatic synthesis of a branching decasaccharide fragment of the capsular polysaccharide of type III Group B Streptococcus. Bioorg. Med. Chem. Lett. 1991, 1, 991-394 and Cattaneo, V.; Carboni, F.; Oldrini, D.; Ricco, R. D.; Donadio, N.; Ros, I. M. Y.; Berti, F.; Adamo, R., Synthesis of Group B Streptococcus type III polysaccharide fragments for evaluation of their interactions with monoclonal antibodies. Pure and Applied Chemistry 2017, 89(7), 855-875) where the position 3 of Gal was glycosylated with N-phtalimido protected GlcN thiolgycosides in the presence of 0-acetyl in the position 4 during the synthesis GBS PSIII fragments.

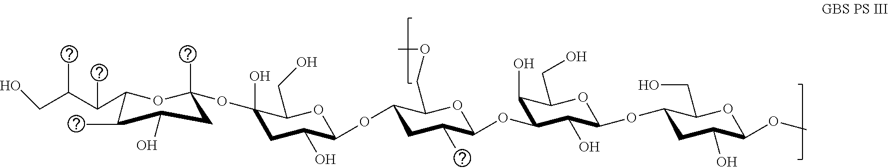

Demechenko et al (A Highly Convergent Synthesis of a Complex Oligosaccharide Derived from Group B Type III Streptococcus. J. Org. Chem. 2001, 66 (8), 2547-2554) synthesized an heptasaccharide portion of GBS PSIII by regioselective glycosylation of the Gal unit from a lactose acceptor, however this GalNAc moiety was already substituted at position 4 with a NeuNAc-α-(2→3) Gal branching. The possibility of regioselectively glycosylating the position 3 of Gal in the presence of 4-OH has been described also for the synthesis of fragments from Pn14, although in this case the use of a 3,4,6-tri-O-acetylated GlcNAc donor did not foresee further chemical elongation of this residue. A similar approach was used for the preparation of a pentasaccharide from N. meningitidis LOS, where the sialic acid was inserted by enzymatic reaction with the deprotected Pn14-like fragment (Yan, F.; Wakarchuk, W. W.; Gilbert, M.; Richards, J. C.; Whitfield, D. M., Polymer-supported and chemoenzymatic synthesis of the Neisseria meningitidis pentasaccharide: a methodological comparison. Carbohydr. Res. 2000, 328, 3-16).

For the synthesis of PSIa related glycans protection of the 4-OH of Gal has typically been used. Recently Guo et al. (Mondal, P. K.; Liao, G.; Mondal, M. A.; Guo, Z., Chemical synthesis of the repeating unit of type Ia group B Streptococcus capsular polysaccharide. Org Lett 2015, 17 (5), 1102-5) used a 4,6-O-benzylidene protection in the Gal acceptor for glycosylation with a GlcN trichloroacetimidate donor, to be subjected to regioselective ring opening before further glycosylation of position 4 for the construction of the trisaccharide GlcNAc-β-(1→3)-[Glc-β-(1→4)] Gal.

We have now envisaged in the regioselective glycosylation of Gal position 3 a method for accelerating the synthesis of the GlcNAc-β-(1→3) Gal disaccharide and render the 4-OH available for glycosylation. This approach could be used to build up a convergent synthetic route towards defined fragments from PSIa, Ib and III.

In this respect, we have found that the role of protective groups is fundamental to control the regioselectivity of the reaction, tuning the relative reactivity of acceptor and donor by the arming or disarming effect and determining the stereochemical orientation of the linkage originated between two sugars.

### SUMMARY OF THE INVENTION

In a first aspect, the invention refers to a compound of formula:

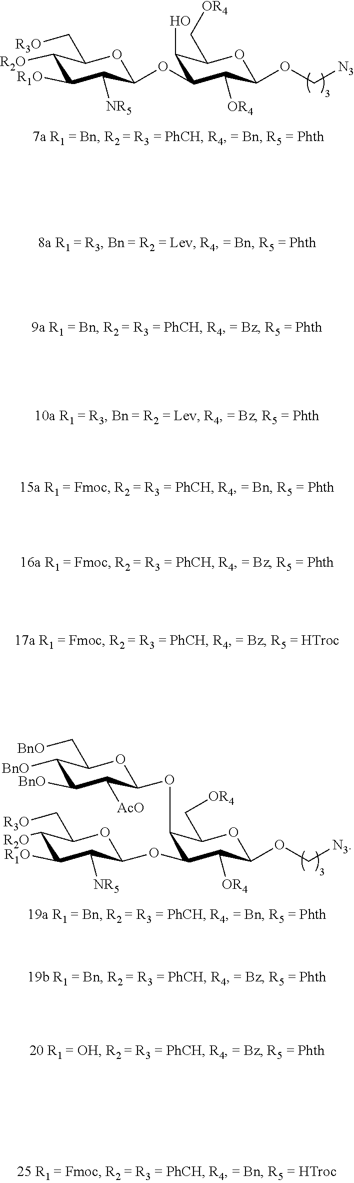

Unless otherwise provided, in the above indicated formulae, R2=R3=PhCH means that the groups R2 and R3 are both connected to the corresponding oxygen to give an acetal moiety of formula:

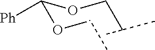

wherein the dotted lines indicate the attachment to the carbon atom of the sugar.

Preferably, the invention refers to compounds of formula:

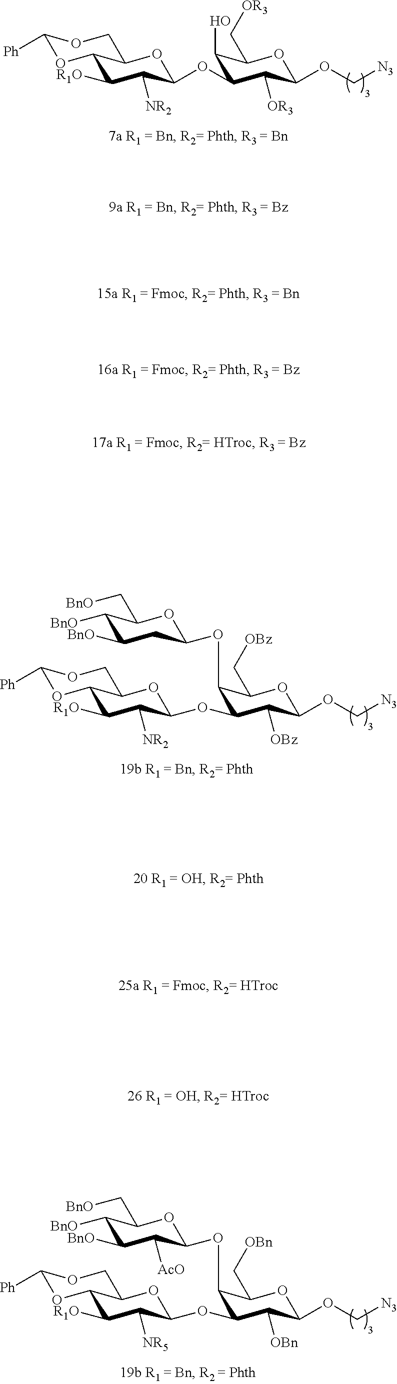

In particular, the above identified disaccharides of formula 7a and 9a are useful as intermediates for the preparation of the trisaccharides of formula 19a and 19b respectively, being these latter useful intermediates for the preparation of fragments from Group B Streptococcus (GBS) capsular polysaccharide type Ia.

The above identified disaccharide of formula 16a and 17a are useful as intermediates for the preparation of the trisaccharides of formula 25 and 26, being these latter useful intermediates for the preparation of fragments from Group B Streptococcus (GBS) capsular polysaccharide type Ib.

According to a further aspect, the invention refers to conjugated derivatives comprising oligosaccharide synthesized via the above indicated trisaccharides of formula 19a, 19b, 20, 25 and 26 connected to a carrier protein, preferably CRM 197. The invention also comprises the use of said conjugates in the preparation of portion of GBS PS Ia, Ib and III, having different length or different number of repeating units.

In this respect, the invention also refers to a process for the preparation of fragments from Group B Streptococcus (GBS) capsular polysaccharide type Ia, Ib, III comprising the polymerization of the above indicated trimer repeating unit, optionally conjugated to a carrier protein.

### BRIEF DESCRIPTION OF THE DRAWINGS

FIG. 1: ¹H NMR spectrum of compound 32a.

FIG. 2: ¹H NMR spectrum of compound 37.

FIG. 3: 1H NMR spectrum of compound 36.

FIG. 4A: Characterization of glycoconjugate 32a-CRM197. SDS Page electrophoresis. 1. branched-CRM197 ˜2.5 mol/mol ratio; 2. branched-CRM197˜20 mol/mol ratio; 3.32a-CRM197

FIG. 4B: Characterization of glycoconjugate 32a-CRM197. Western blot with anti GBS PSIII murine serum.

### DETAILED DESCRIPTION

The present invention provides a defined method for the selective preparation of the repeating units of the GBS polysaccharides Ia, Ib and III, using specific protective groups pattern that allows the regioselective glycosylation at 3-OH of the Galactose (Gal) moiety. The 4-OH position was therefore available for glycosylation with Glucose (Glc), allowing the synthesis of PS Ia/b fragments, which cannot be obtained by depolymerisation of the entire polysaccharide. In addition, this strategy can give access to polysaccharide derivatives such as the hexa- and octasaccharide from PSIII, covering the recently identified epitope.

In other words, the present invention generally refers to regioselective synthesis of GlcNAc-β-(1→3) Gal building blocks, for construction of fragments from Group B Streptococcus (GBS) capsular polysaccharide type Ia, Ib and III, useful for the preparation of polymeric derivatives covering the epitope of the indicated GBS polysaccharides.

Advantageously over the prior art, the present invention allows for the preparation of portions of GBS PS Ia, Ib and III antigens with a reliable and more convenient synthetic process, which encompasses the use of building blocks able to undergo selective regiospecifical functionalization to obtain the final polysaccharides in high yield and avoiding protection/de-protection steps as so far necessary according to the prior art synthetic approaches.

Unless otherwise indicated, the skilled person will recognize that all the abbreviations used in the description regarding the protecting groups are well known in organic synthesis. As general reference on protecting group, se e.g. Peter G. M. Wuts: “Greene's protective groups in organic synthesis”.

As an example, the following table lists some of the protecting groups as herein indicated:

TABLE 1
Protecting Groups

| Abbreviation | Chemical name | Formula |
|---|---|---|
| Lev | Levulinoyl ester | CH₃CO CH₂CH₂CO— |
| Phth | N-phthalimido | 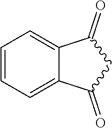 |
| Troc | trichloroethoxycarbonyl | Cl₃CCH₂NHCO— |
| Bn | Benzyl | 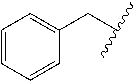 [SMILES: *Cc1ccccc1] |
| Bz | Benzoil | 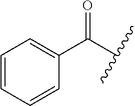 [SMILES: *C(=O)c1ccccc1] |
| Fmoc | Fluorenylmethylocycarbonyl | 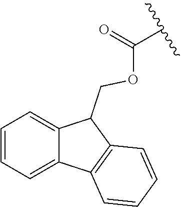 [SMILES: *C(=O)OCC1c2ccccc2-c2ccccc21] |
| OTf | Triflate | CF₃SO₂O— |

Unless otherwise indicated, the indication “PS” means polysaccharides.

Preferably, the invention refers to a polysaccharide of formula 19a and 19b for the preparation of repeating unit of GBS PS Ia.

Preferably, the invention refers to a polysaccharide of formula 25 and 26 for the preparation of the repeating unit of GBS PS Ib. Of note, the intermediate 25 may also be used for the preparation of the repeating unit of GBS PS Ia as well as PSIa.

Preferably, the invention refers to a polysaccharide of formula 16a and 17a for the repeating unit of GBS PS Ib.

Preferably, the invention refers to a polysaccharide of formula 9a and 10a for the preparation of the repeating unit of GBS PS Ia.

According to one embodiment of the invention, the regioselective glycosylation at the position 3 occurs according to the following retro synthetic Scheme A:

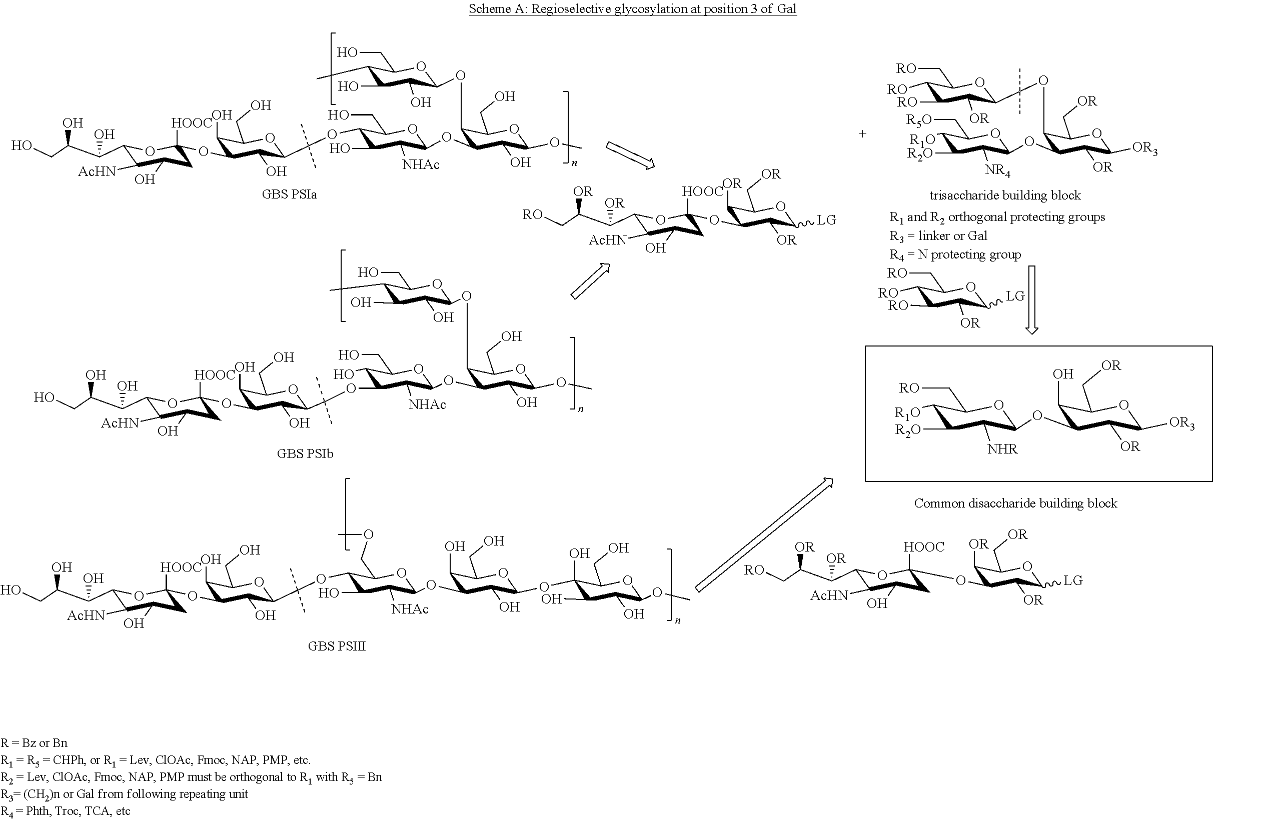

A shown in the above Scheme A, GBS PSIa and Ib repeating units differs for the connection of the NeuNAc-α-(2→3)-Gal with the trisaccharide GlcNAc-β-(1→3)-[Glc-β-(1→4)]Gal, which is β-(1→4) and β-(1→3), respectively. Taking this into account, the two structures can by synthesized from a common trisaccharide intermediate with an appropriate combination of orthogonal protecting groups at positions 3 and 4 of the Glucosamine (GlcN). The trisaccharide acceptor could in turn derive from regioselective glycosylation of the Gal 3-OH with a GlcN donor. The availability of a GlcNAcβ(1→3) Gal disaccharide building block is key to this approach. In addition, the regioselective β-(1→3) insertion of GlcN on Gal could offer access to fragments of PSIII longer than those recently reported (see e.g. Pozsgay, V.; Gaudino, J.; Paulson, J. C.; Jennings, H. J., Chemo-enzymatic synthesis of a branching decasaccharide fragment of the capsular polysaccharide of type III Group B Streptococcus. Bioorg. Med. Chem. Lett. 1991, 1, 991-394, and Demchenko, A. V.; Boons, G.-J., A Highly Convergent Synthesis of a Complex Oligosaccharide Derived from Group B Type III Streptococcus. J. Org. Chem. 2001, 66 (8), 2547-2554).

To achieve regioselective glycosylation of Gal with an appropriate GlcN donor, we focused on the effect of arming benzyl and disarming benzoyl groups at position 2 and 6 of Gal 2 in tuning the reactivity of the 3- and 4-OH, respectively as illustrated in Scheme B. To this end we initially explored a series of GlcN thioglycoside or trichloroacetimidate donors with the amine protected by a phthalimido, a trichlorethylcarbamate or a trichloroacetyl protection. Lev and Fmoc were selected for protection of either position 3 or 4. Alternatively 4,6-O-benzylidene was used to lock the 4 and 6 hydroxyls to be subjected to regioselective ring opening delivering the 4-OH at a later stage of the synthesis.

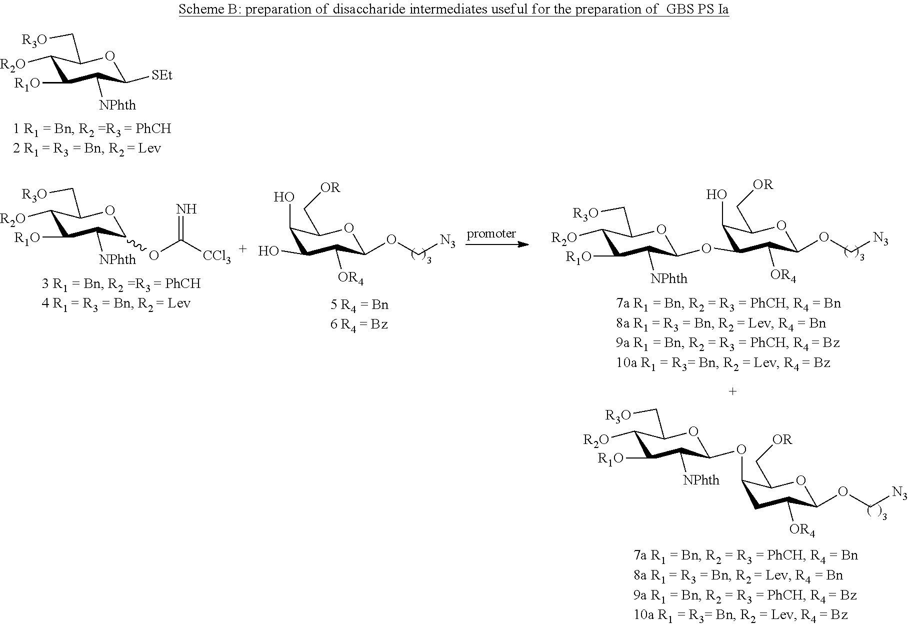

TABLE 2
Reaction of GlcN donors 1-4 with Gal acceptor 5-6

| Entry | Donor | Acceptor | Reaction Conditions | Products (Yields) |
|---|---|---|---|---|
| 1 | 1 | 5 | NIS/TfOH, −30° C. | nd^{a} |
| 2 | 1 | 6 | NIS/TfOH, −30° C. | nd^{a} |
| 3 | 1 | 5 | NIS/Ag(OTf), −30° C. | 7a (43%), 7b (26%) |
| 4 | 1 | 6 | NIS/Ag(OTf), −30° C. | 9a (53%) |
| 5 | 2 | 5 | NIS/Ag(OTf), −30° C. | 8a (40%), 8b (28%) |
| 6 | 2 | 6 | NIS/Ag(OTf), −30° C. | 10a (65%) |
| 7 | 3 | 5 | TMSOTf, −10° C. | 7a (31%) |
| 8 | 3 | 6 | TMSOTf, −10° C. | 9a (64%) |
| 9 | 4 | 5 | TMSOTf, −10° C. | 8a (45%) |
| 10 | 4 | 6 | TMSOTf, −10° C. | 10a (33%) |

^{a}nd = not determined, product could not be detected.

When the ethylthioglycoside 1 was tested with acceptors 5 and 6 using NIS/TfOH as promoters in DCM at −30° C., no product formation was observed (Entry 1-2, Table 2) due to donor decomposition. We therefore decided to use a milder Lewis acid, and NIS/AgOTf at −30° C. was tried (Entry 3, Table 2). Under these conditions the 4,6-O-benzylidene thioglycoside 1 afforded the desired product 7a in 43% together with the 4-O-glycosylated product 7b (28%). Similarly the 4-O-Lev thioglycoside 2 gave (Entry 5, Table 2) gave a 4:3 molar ratio of the β(1→3) 8a and β(1→4) 8b linked disaccharides. The formation of the corresponding GlcNAc-β-(1→4)-Gal disaccharide was confirmed by acetylation of the secondary product. In the ¹H NMR spectrum of this compound a shift from 3.32 to 4.69 of the H-3 signal of Gal, appearing as a doublet of doublets with J_{2,3}=10.3 Hz and J_{3,4}=2.5 Hz and indicated that the glycosylation had occurred at position 4.

Imidate donors were tested as alternative to thioglycosides Reaction of 5 with the 4,6-O-benzylidene GlcN trichloloroacetomidate 3 in presence of TMSOTf in DCM at −10° C. (Entry 7, Table 2) provided the desired product 7a in 31% yield, due to concomitant formation of the anomeric acetamide byproduct from the donor. On the other hand, the imidate 5 gave exclusive formation of 8a with higher yield (45%, Entry 9, Table 2), highlighting that combination of 4,6-O-benzylidene protection and trichloroacetidimoyl leaving group resulted in the best reaction outcome.

When acceptor the di-O-benzoyl acceptor 6 was exploited, reaction with donor 1 under NIS/AgOTf activation afforded regioselectively compound 9a (53%, Entry 4, Table 2). A good yield was obtained in the same conditions with donor 2 to give 10a (Entry 6, Table 2). The imidate 4 (Entry 10, Table 2) also gave compound 10a, but in a lower yield (33%) compared to the 4,6-O-benzylidene 3 (Entry 8, Table 2) which gave 9a in 64% yield.

To summarize, these findings indicated that 2,6-di-OBz Gal acceptor generally lead to higher regioselectivity and yields compared to the 2,6-di-OBn derivative. Trichloroacetimidate donors 3 and 4 showed higher regioselectivity with both the 2,6-di-OBz and 2,6-di-O-Bn Gal acceptors, but with variable yields. The most efficient routes to GlcNAc-β-(1→3)Gal were achieved by combination of the 2,6-di-OBz acceptor 5 with either 4-O-levulinoyl ethylthiol 2 by NIS/AgOTf mediated activation or the 4,6-O-benzylidene GlcN imidate 3 in presence of TMTSOTf, which lead to disaccharides 10a and 9a, respectively. Furthermore, the 3,6-di-O-benzyl ether 2 with NIS/AgOTf activation performed better than the TCA counterpart 4.

These results can be rationalized considering that the regioselectivity of the reaction benefits of the more pronounced electron withdrawing effect of the 2,6-O-benzoyl as compared to 2,6-di-O-benzyl substituents in the Gal acceptor. The benzoyl groups further decrease the intrinsically lower nucleophilicity of the axial 4-hyodroxyl respect to the 3-hydroxyl group. In addition, mild activation conditions (NIS/AgOTf) for the thiolglycoside donor or the torsional disarming effect of the benzylidene group for the trichloroacetimidate donor appear to favor the glycosylation reaction over donor decomposition.

Having identified conditions to obtain a selective β-(1→3)glycosylation with GlcN donors bearing a temporary group at position 4, the same strategy was transferred to GlcN donors with an orthogonal protection at position 3, in order to obtain a disaccharide that can be elongated at that position, useful for the preparation of the repeating unit of the GBS PS Ib. With reference to Scheme C, Fmoc was introduced at the 3-OH of GlcN, and corresponding ethylthiol protected as N-pthalimido 11 and N-trichloroethoxycarbamyl 12 derivatives were tested as donors for 5 and 6. N-troc protected GlcN was also tested as trichloroacetimidate donor, considering the good selectivity reached with this class of donors in the previous set of reactions.

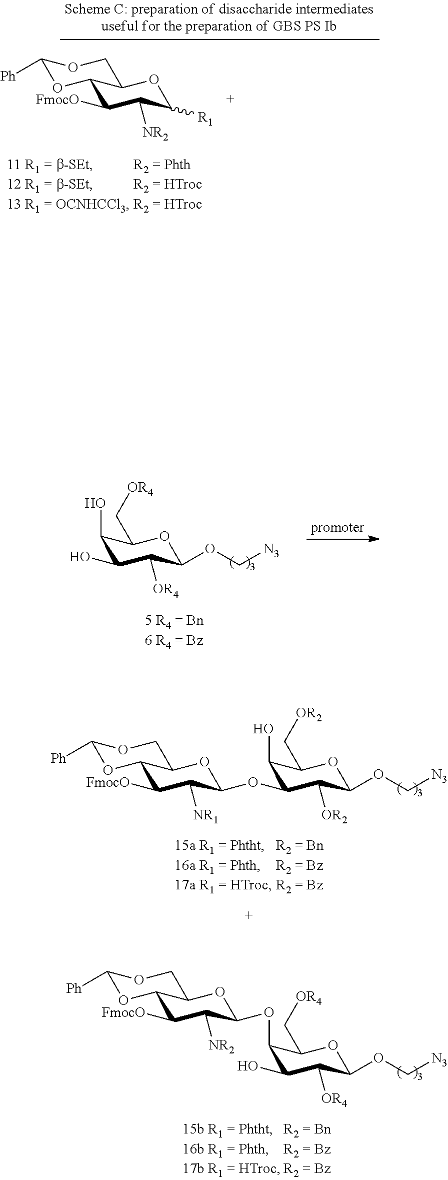

TABLE 3
Reaction of GlcN donors 11-13 with Gal acceptors 5-6.

| | Donor | Acceptor | Reaction Conditions | Products (Yields) |
|---|---|---|---|---|
| 1 | 11 | 5 | NIS/TfOH, −30° C. | 15a (30%), 15b (<5%) |
| 2 | 11 | 5 | NIS/AgOTf, −30° C. | 15a (38%), 15b (26%) |
| 3 | 11 | 6 | NIS/TfOH, −30° C. | 16a (40%) |
| 4 | 11 | 6 | NIS/AgOTf, −30° C. | 16a (68%) |
| 5 | 12 | 6 | NIS/TfOH, −30° C. | nd^{a} |
| 6 | 12 | 6 | NIS/AgOTf, −30° C. | 17a (65%) |
| 7 | 13 | 6 | TMSOTf | 17a (70%) |

^{a}nd = not determined, product could not be detected.

The glycosylation of di-O-benzyl acceptor 5 with donor 11 using NIS with either TfOH or AgOTf as promoters gave mixtures of the β-(1→3) 15a and β-(1→4) 15b disaccharides (Entry 1-2, Table 3). Again, switch to the di-O-benzoyl acceptor 6 in presence of NIS/TfOH allowed to obtain the desired product 16a (40%, Entry 3, Table 3) in mixture with a non identified byproduct. The use of NIS/AgOTf at −30° C. further improved the yield up to 63% (Entry 4, Table 3), confirming a better capacity of the benzoyl substituents to orient the regioselectivity of the reaction. These conditions were proven efficient also for NTroc donor 12 which gave 17a in 65% yield (Entry 6, Table 3). When the corresponding trichloroacetimidate 13 was exploited, the yield was increased to 70% (Entry 7, Table 3), corroborating the potential of this type of donors for the regioselective control of the reaction.

Glycosylation of Gal at Position 4

Having set up conditions to access regioselectively to the GlcNAc-β-(1→3)-Gal motif, the possibility to glycosylate the 4-OH on the Gal unit was explored (Scheme 1).

While the reaction of the peracetylated trichloroacetimidate 17 and disaccharide 9a did not take place, the armed donor 18 gave thrisaccarides 19a and 19b in 73% and 65% yield, respectively. In this case despite the deactivating effect of the 6-O-benzoyl ester compared to the 6-O-benzyl ether in the reactivity of the Gal 4-OH, reaction proceeded with almost equal efficiency.

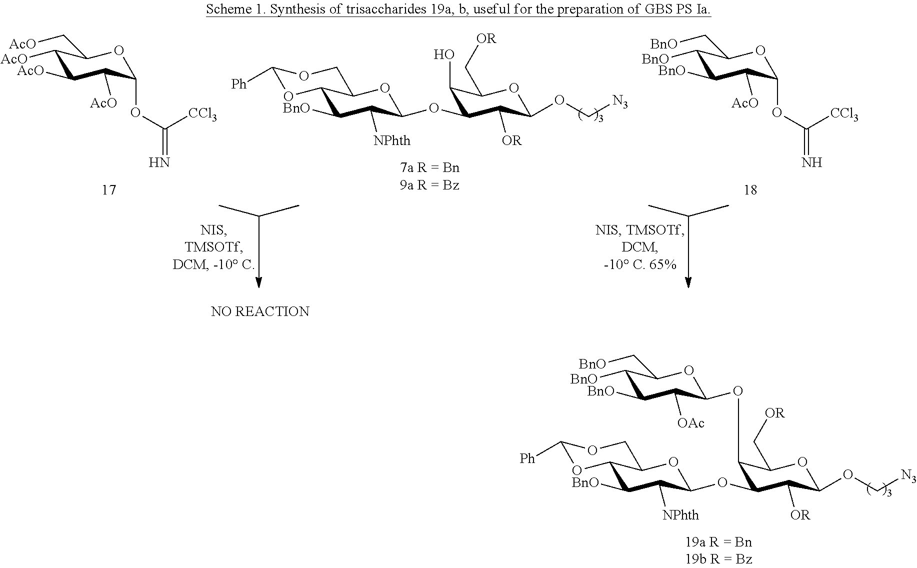

The trisaccharide motifs so far synthesized could be further extended to form the full repeating units of GBS type Ia and b polysaccharides. To test this hypothesis compound 19b was subjected to selective opening of the benzylidene ring to provide in 70% yield the 4-OH acceptor 20, which was elongated with the known sialylated disaccharide 21 (see Cattaneo, V.; Carboni, F.; Oldrini, D.; Ricco, R. D.; Donadio, N.; Ros, I. M. Y.; Berti, F.; Adamo, R., Synthesis of Group B Streptococcus type III polysaccharide fragments for evaluation of their interactions with monoclonal antibodies. Pure and Applied Chemistry 2017, 89(7), 855-875) under TMOTf activation to give the pentasaccharide 22, corresponding to the protected GBS PSIa repeating unit as per Scheme 2.

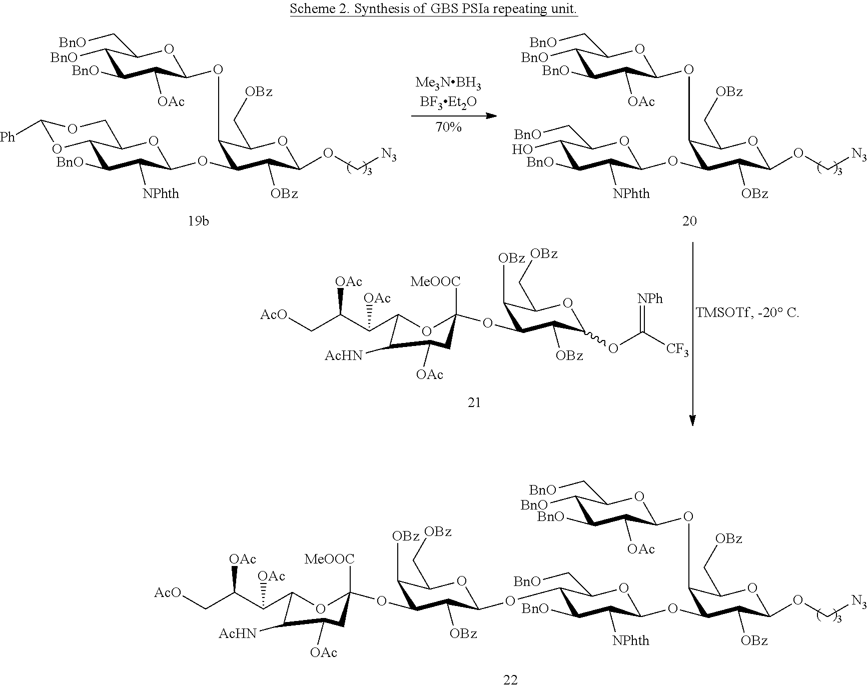

Attempts to use a similar the trisaccharide acceptor with an Fmoc protection at position 3 of the GlcN unit to leading to the assembly of GBS PIb repeating unit failed (Scheme 3). After constructing compound 20 by reaction of disaccharide 15a and the glucosyl donor 23 by NIS/TfOH activation, the Fmoc group was removed by treatment of the formed disaccharide with pyperidine. The trisaccharide acceptor 24 resulted too deactivated for reaction with 21 in the presence of TMSOTf, and no product formation was observed. We therefore anticipated that replacement of the NPhth protection with the corresponding NTroc derivative would result in a higher nucloephilicity of the vicinal 3-OH. Accordingly, the trisaccharide acceptor 26 was assembled by reaction of 17a with 18, followed by Fmoc removal. Glycosylation in this case proceeded to give pentasaccharide 27.

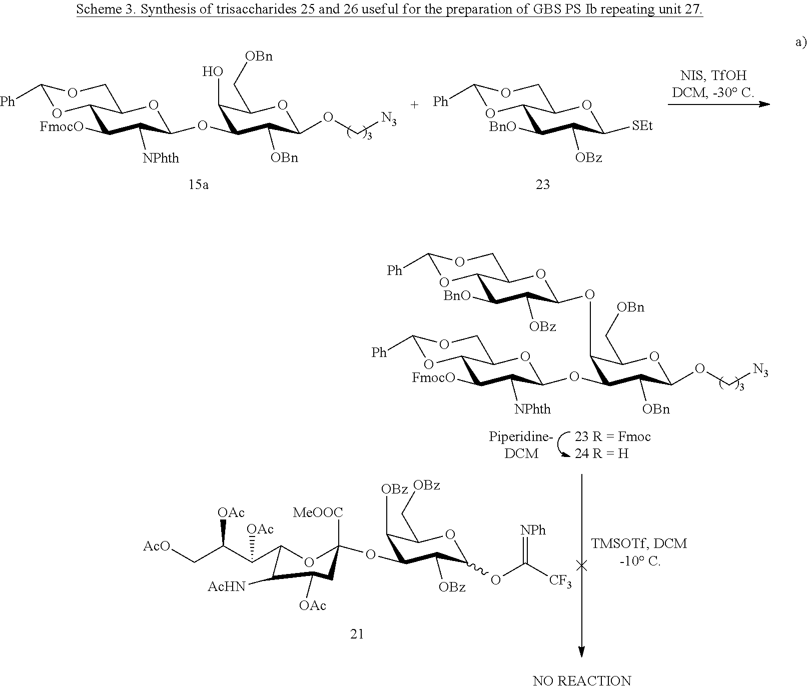

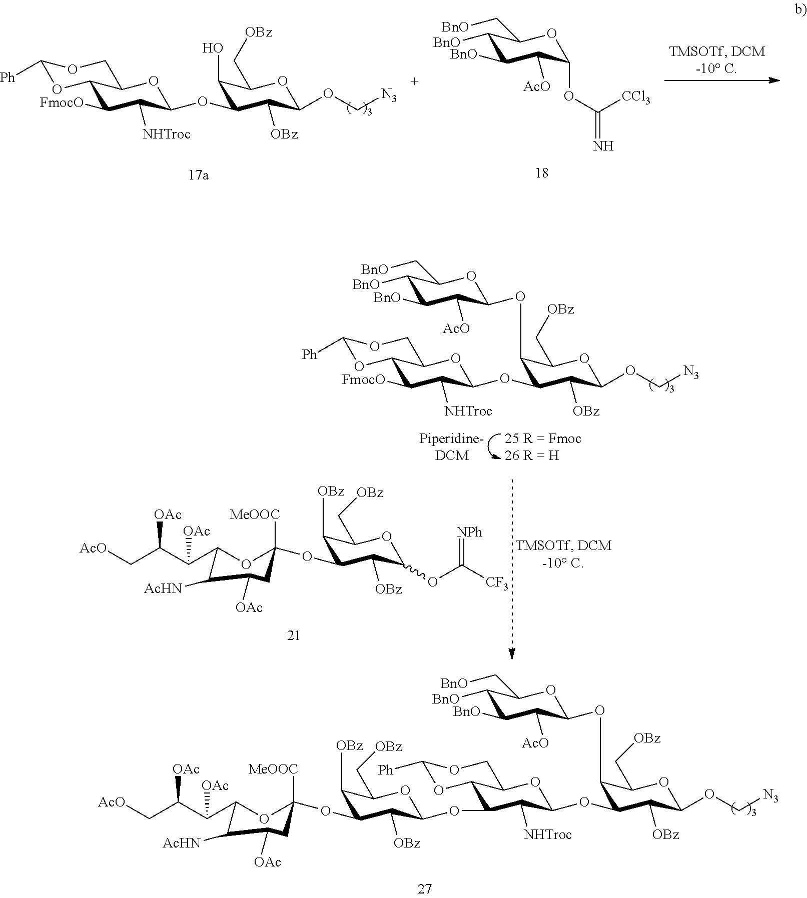

According to a further embodiment of the invention, the described approaches was used also to obtain GBS PSIa and Ib repeating units from a common intermediate (Scheme 4).

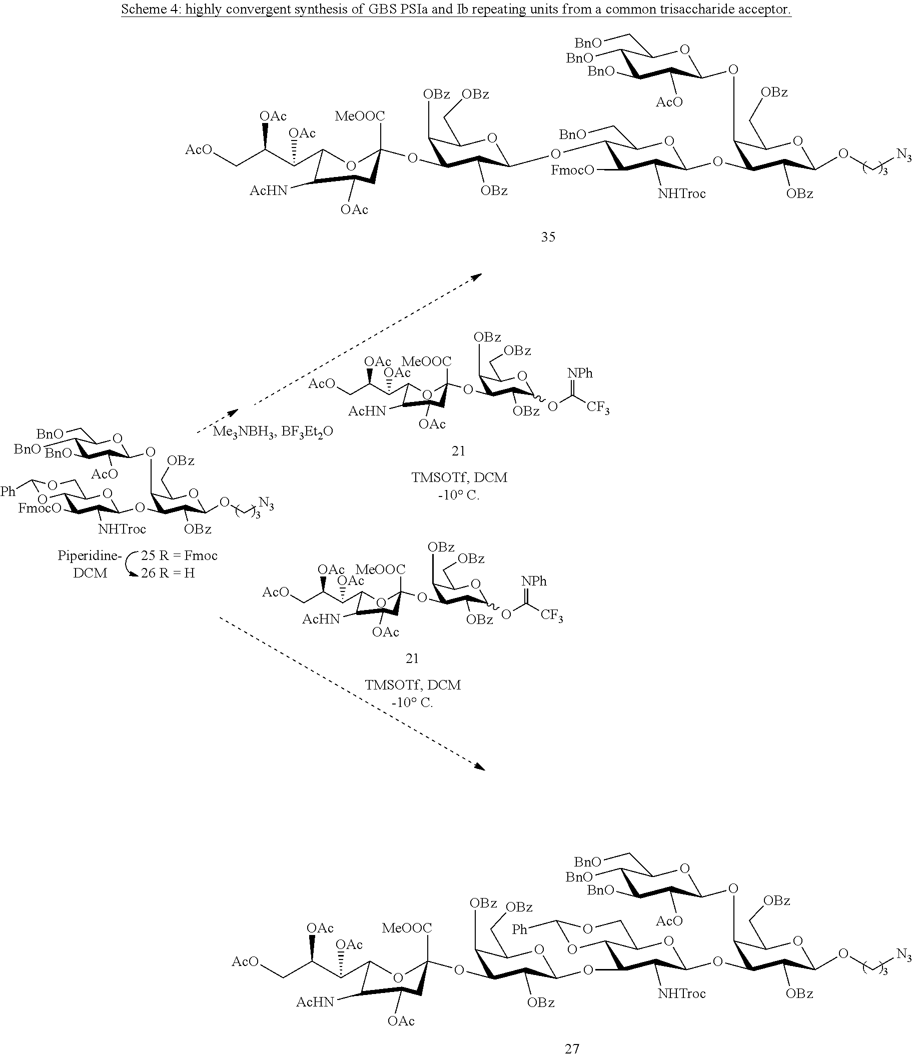

### Elongation of Repeating Units

The regioselective glycosylation of Gal 3-OH could be useful to access larger GBS PS structures as illustrated in Schemes 5 and 6. To proof this concept the repeating unit of GBS PSIII was assembled similarly as described in literature, except that a 3,4-O-protected lactoside (Sundgren, A.; Lahmann, M.; Oscarson, S., Block Synthesis of Streptococcus pneumoniae Type 14 Capsular Polysaccharide Structures*. Journal of Carbohydrate Chemistry 2005, 24 (4-6), 379-391) donor was used for glycosylation of trisaccharide 28, according to Scheme 5.

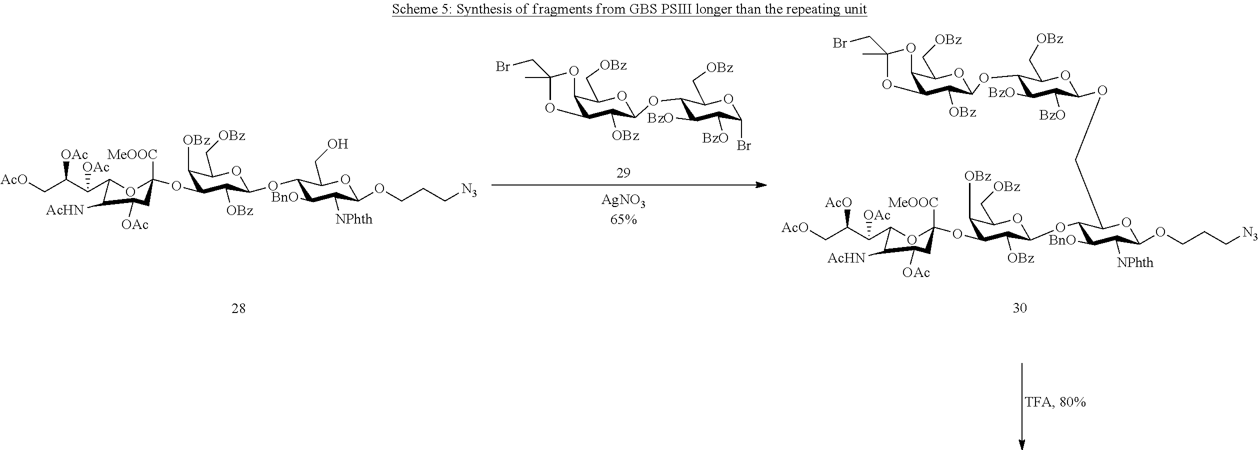

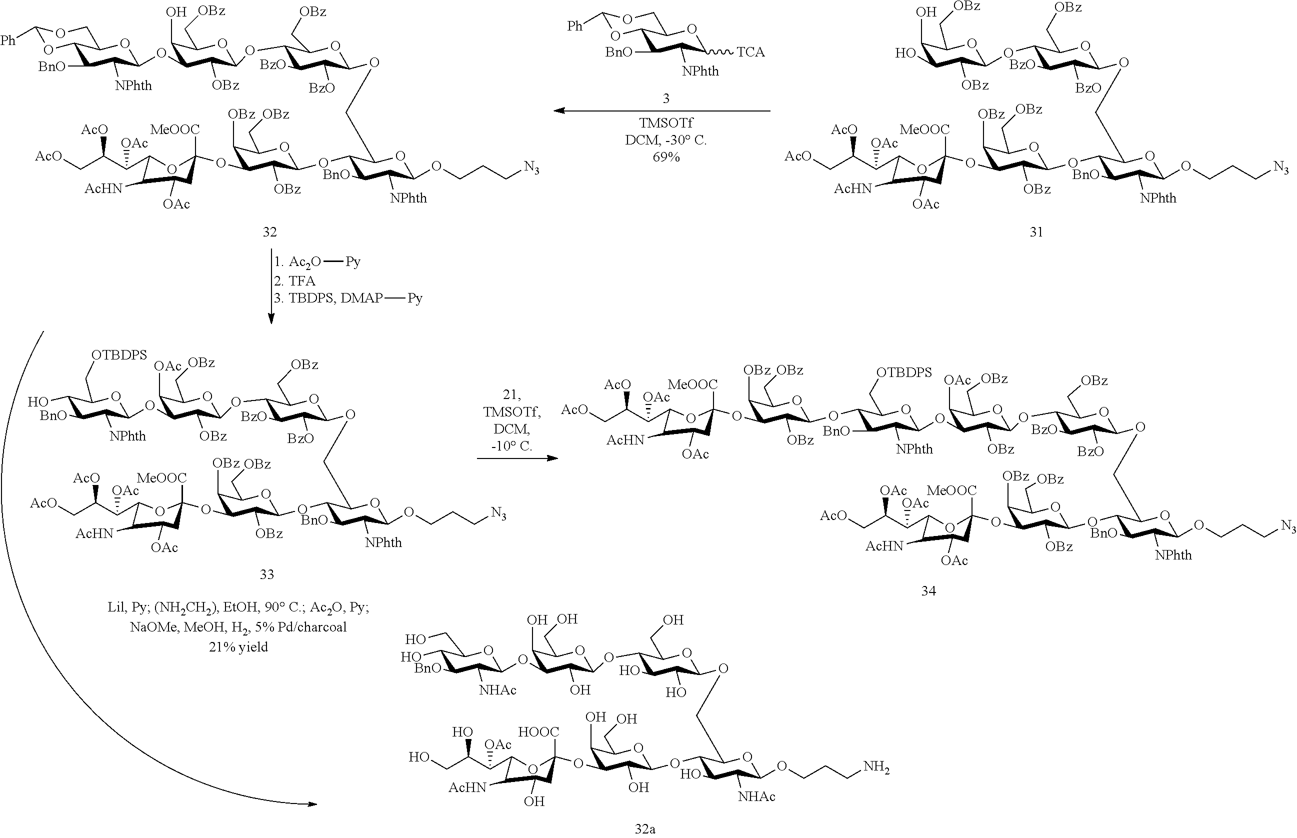

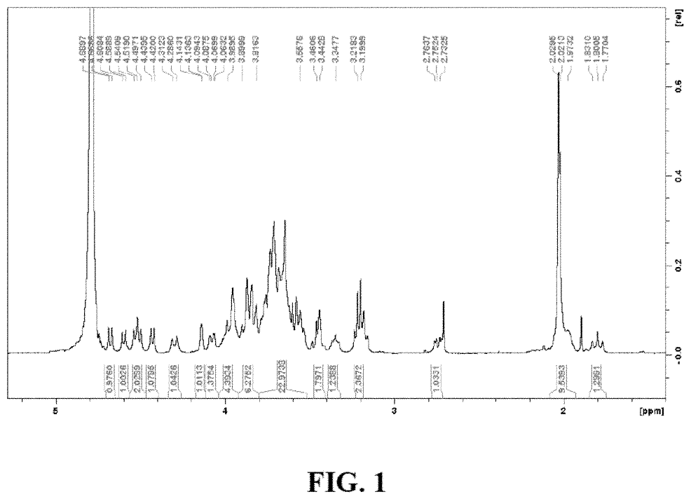

After de-O-isopropylidination of the formed pentasaccharide 31 in 80% yield, regioselective glycosylation with donor 3 afforded the hexasaccharide 32 (69%). Compound 32 was deprotected by a six-step procedure consisting of saponification with Litium iodide in pyridine of the sialic methyl ester moiety; reaction with ethylenediamine in refluxing ethanol for concomitant removal of the O-acyl esters and the phthalimido groups; reacetylation of the intermediate amino oligosaccharide with acetic anhydride in pyridine to install the acetamide group of the GlcpNAc residues; deacetylation with NaOMe/MeOH and final catalytic hydrogenation over 5% Pd/charcoal of the deacetylated product, purified by reverse phase chromatography on a C18 column. The released target hexasaccharide 32a (FIG. 1), where the azide of the spacer has been reduced to amino group, was purified by size exclusion column chromatography on Sephadex G-10, to obtain the final compound in overall 21% yield from 32, as estimated by spectrophotometric quantification of the sialic acid content. FIG. 1 depicts the ¹H NMR spectrum of compound 32a.

Compound 32 was further elongated by acid hydrolysis of the benzylidene protection, followed by selective silylation of the primary alcohol to give 33. Glycosylation of 33 with 21, gave the octasaccharide 34.

Through the develop method a panel of different GBS PSIa and PSIb related fragments can be accessed.

An example of such structures is depicted in Scheme 6.

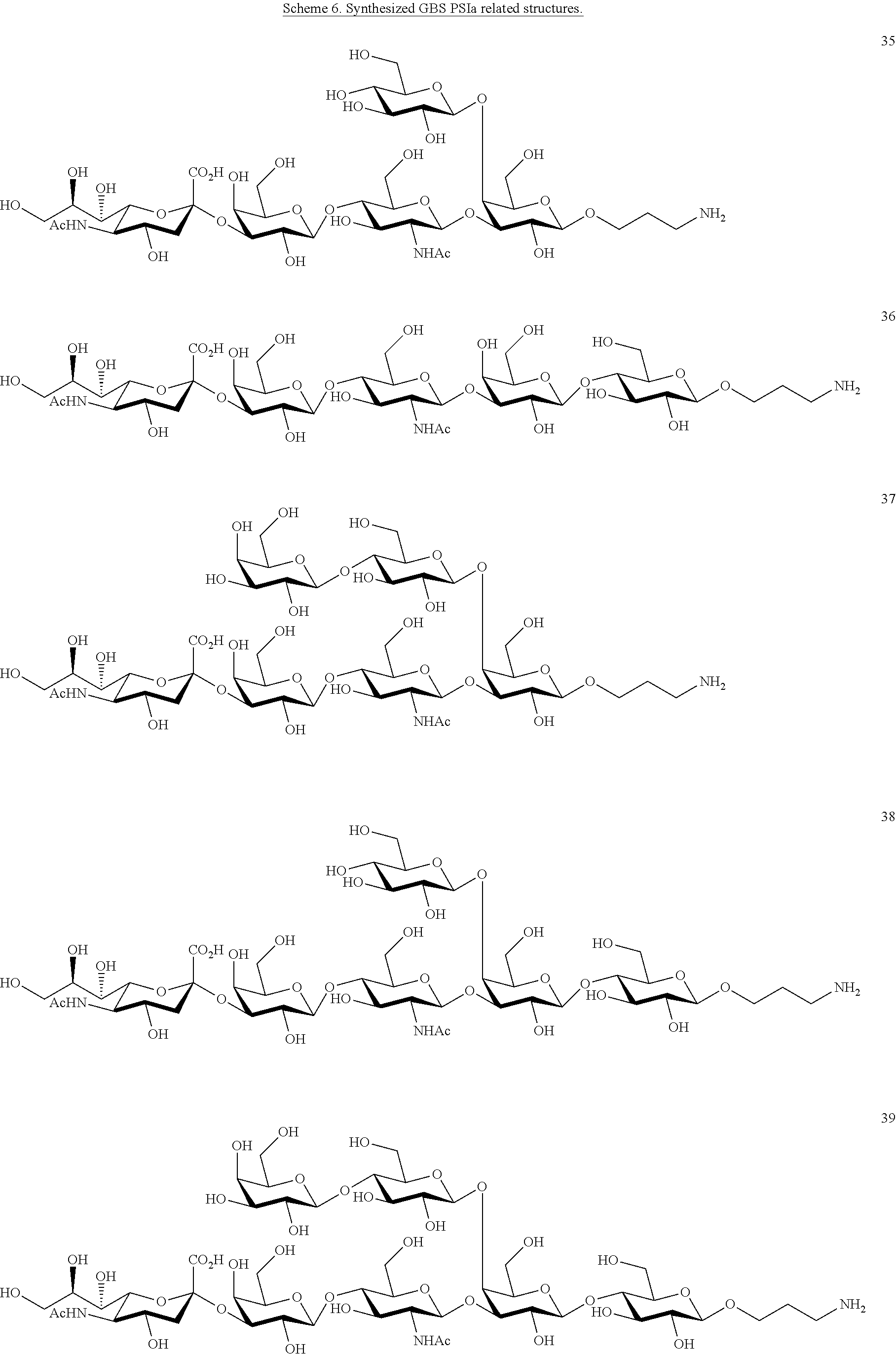

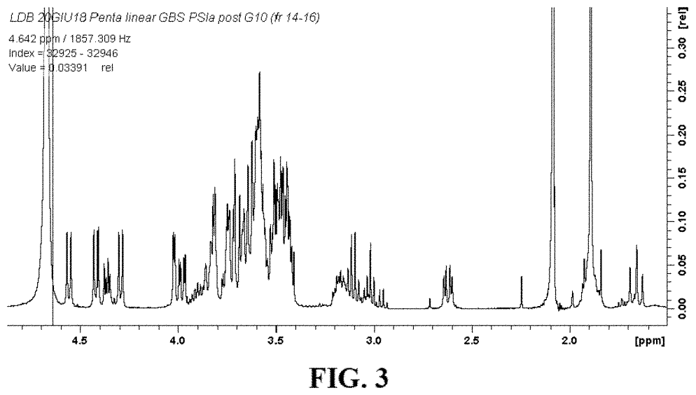

For instance, the key disaccharide 9a can be glycosylated with the lactose donor 40 to obtain the tetrasaccharide 41 (Scheme 7). After regioselective ring opening of the benzylidene group in the GlcN residue, glycosylation with the NeuAc-Gal donor 21 provided the protected hexasaccharide 43, which was subjected to the removal of temporary protections to afford the target oligosaccharide 37 (FIG. 3).

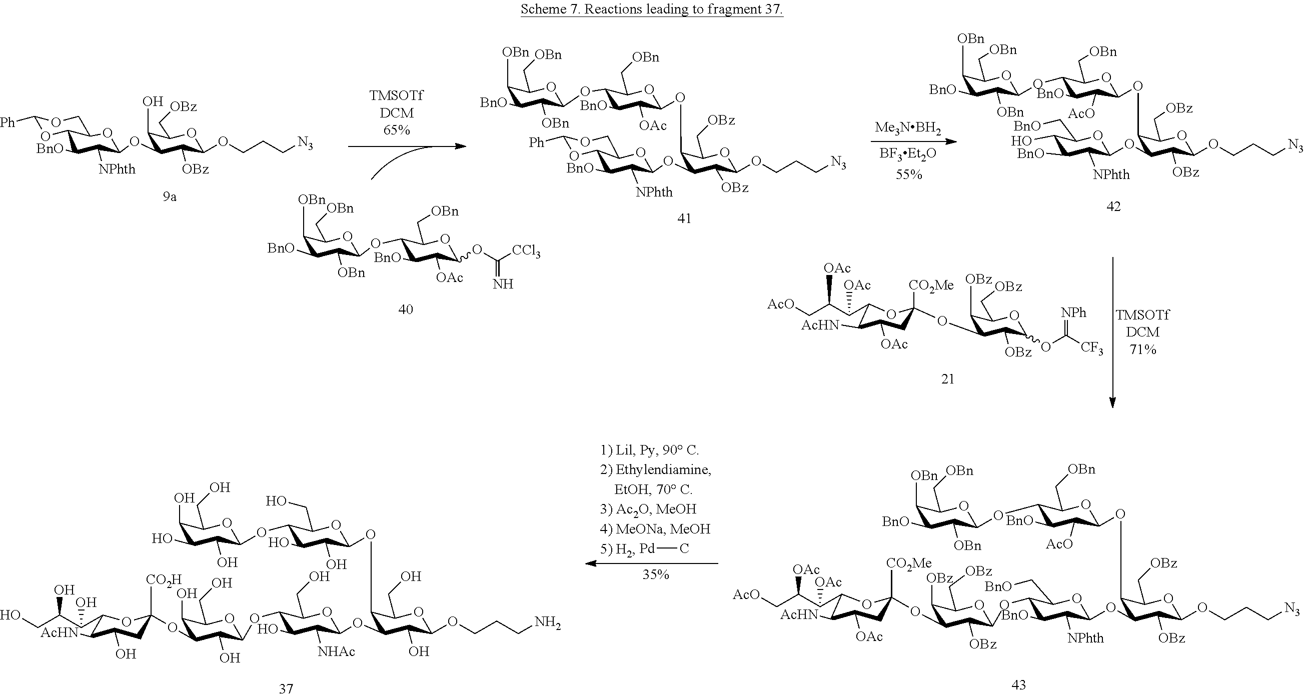

Similarly, regioselective glycosylation of lactose acceptor 44 with GlcN donor 3 provided the trisaccharide 45 which could be in turn glycosylated with the Glc donor 47 obtaining the ramified tetrasaccharide 46. Regioselective ring opening of the benzylidene group and following glycosylation with 21 of the generated hydroxyl group in 47 afforded the hexasaccharide 48, which was deprotected to yield 38.

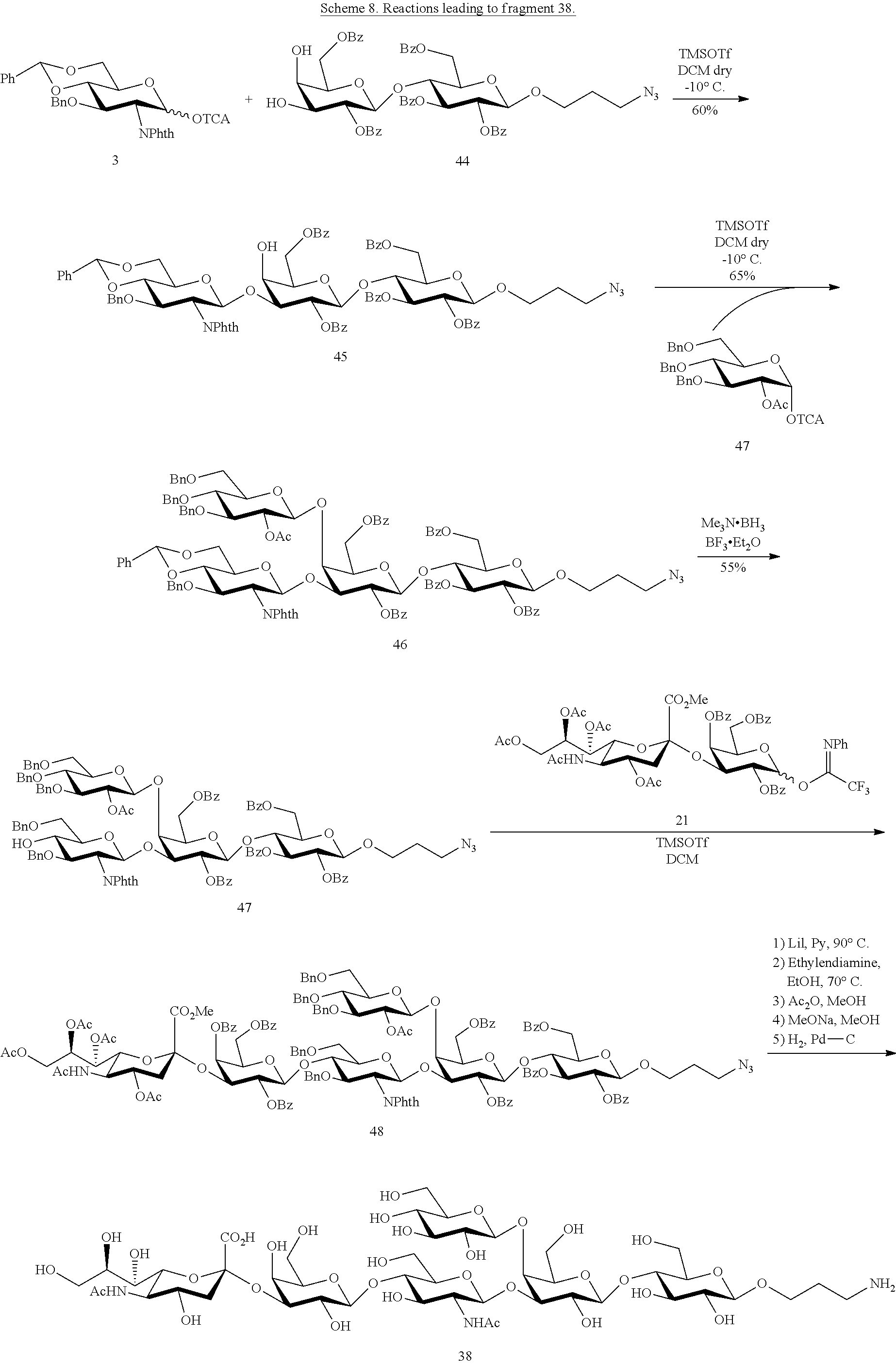

Trisaccharide 45 underwent regioselective ring opening of the benzylidene group for following glycosylation with 21 to give the linear pentasaccharide 47, which was deprotected to provide 36 (Scheme 8, FIG. 3).

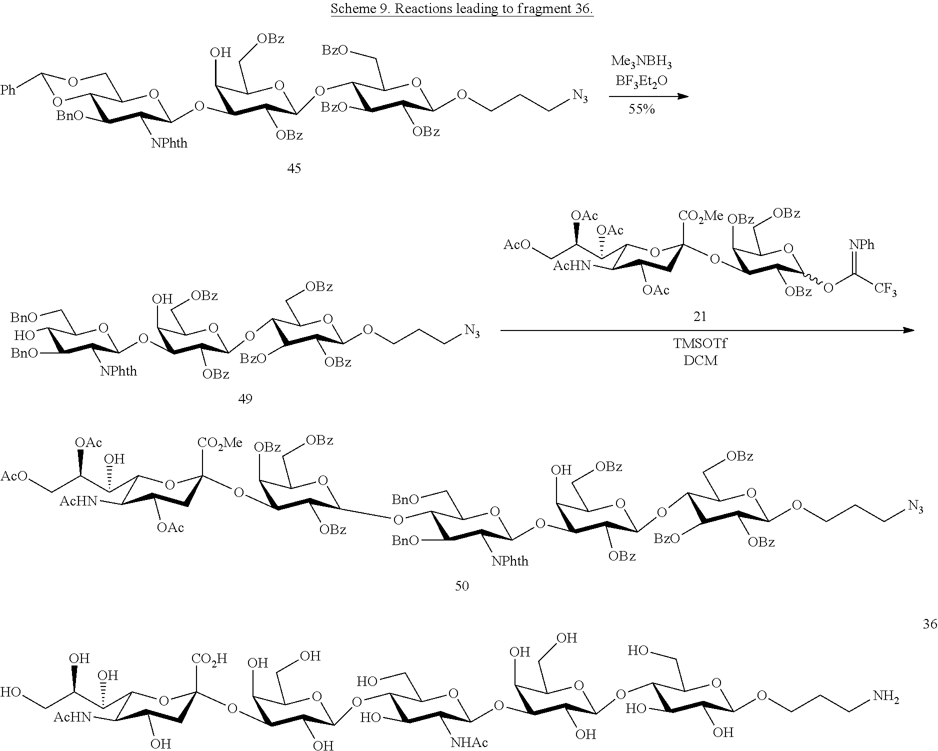

The Scheme 9 illustrates a similar procedure for the elongation of the repeating unit of the GBS PS Ia and Ib, starting from repeating units 35 and 27 respectively.

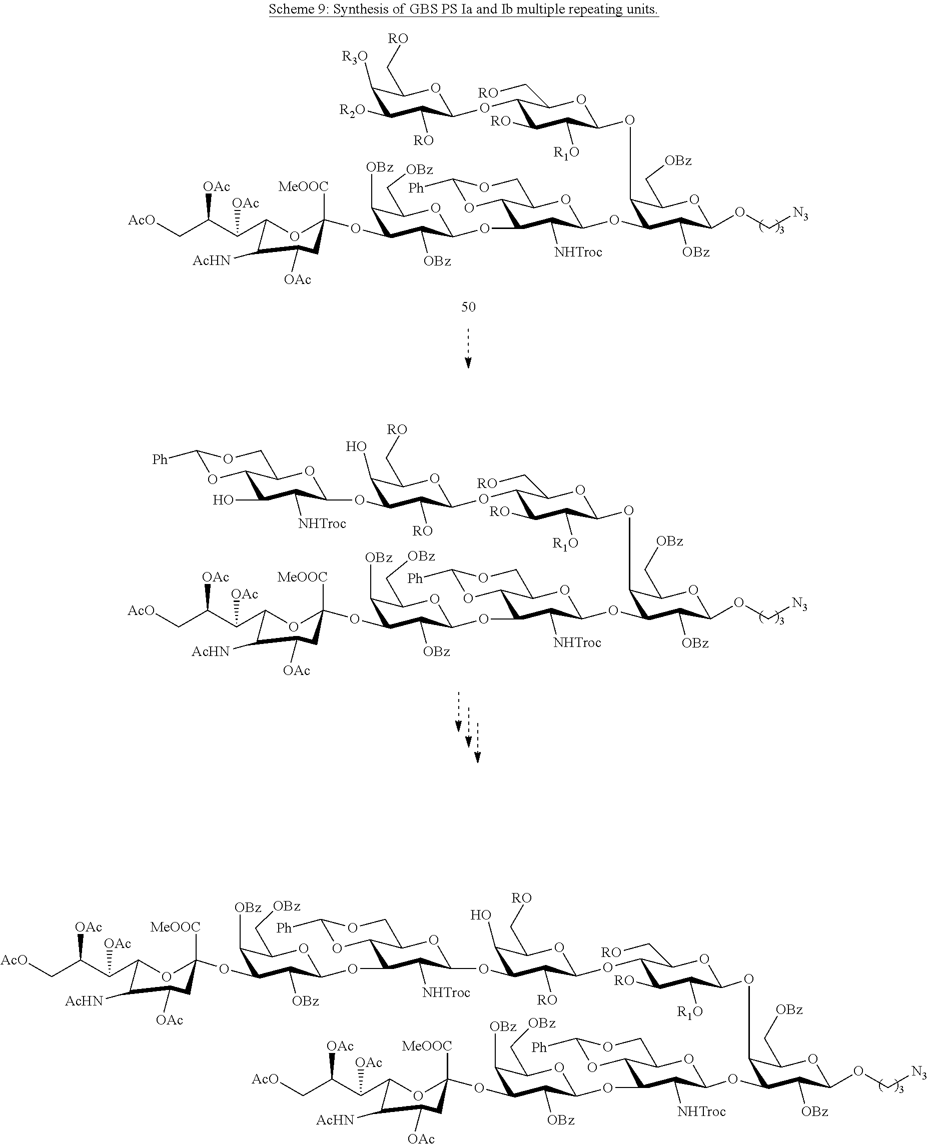

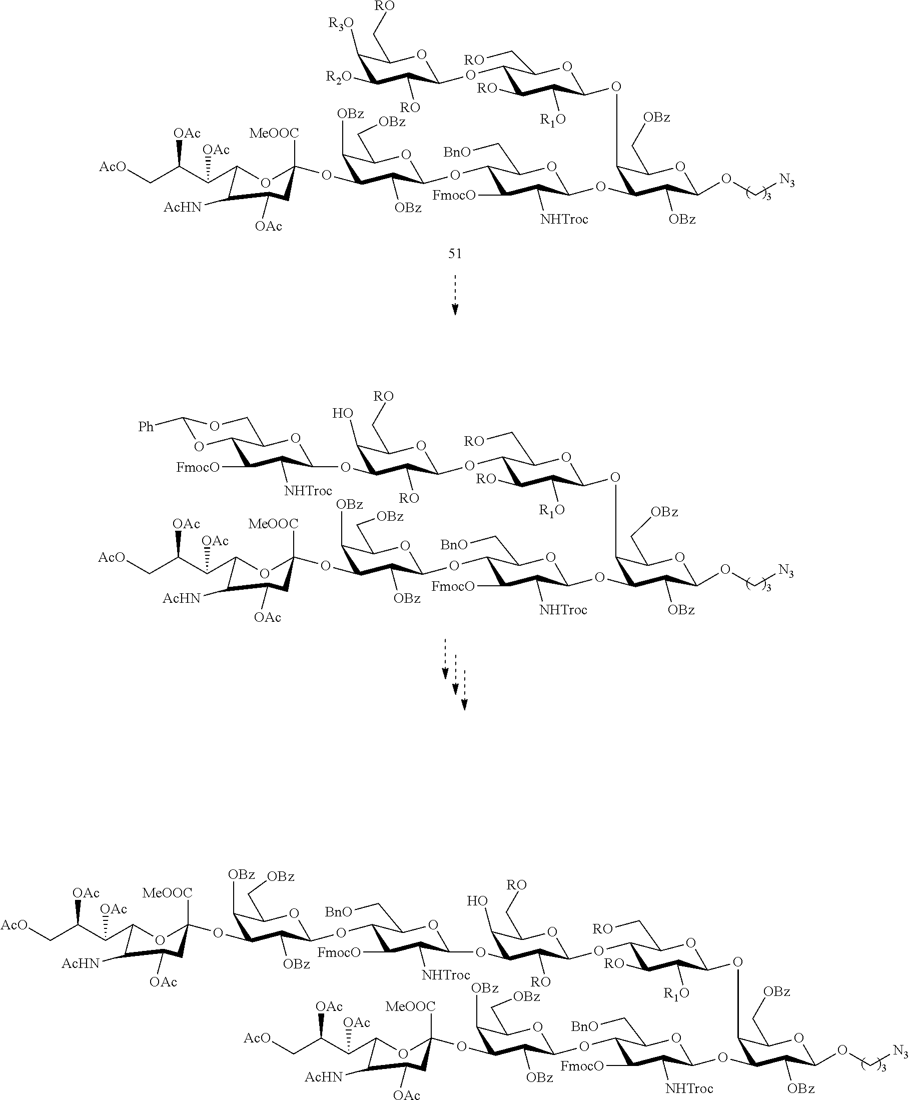

In another embodiment of the invention, the GBS PS Ia, Ib and III fragments obtained according to the present invention longer than one repeating unit could be synthesized by iteration of the developed procedures, as depicted in Scheme 6. Advantageously, the elongation may easily comprise several repeating units, thus rendering the present invention particularly versatile and appreciated by the skilled person. In this direction in fact, it will be possible to choose the proper multiple repeating units fragment, and conjugate it to a carrier protein preferably CRM 197. By that a possible candidate for a vaccine can be obtained and prepared in a very reliable and convenient way as above described in details.

### Conjugation to Carrier Protein

In an additional embodiment, after deprotection according to standard methods, the synthesized structures were connected to carrier proteins through a linker Z, to give the desired conjugated derivatives. As general example, the conjugation may be carried out using procedure known in the art. The following Scheme 7 is an illustration of that:

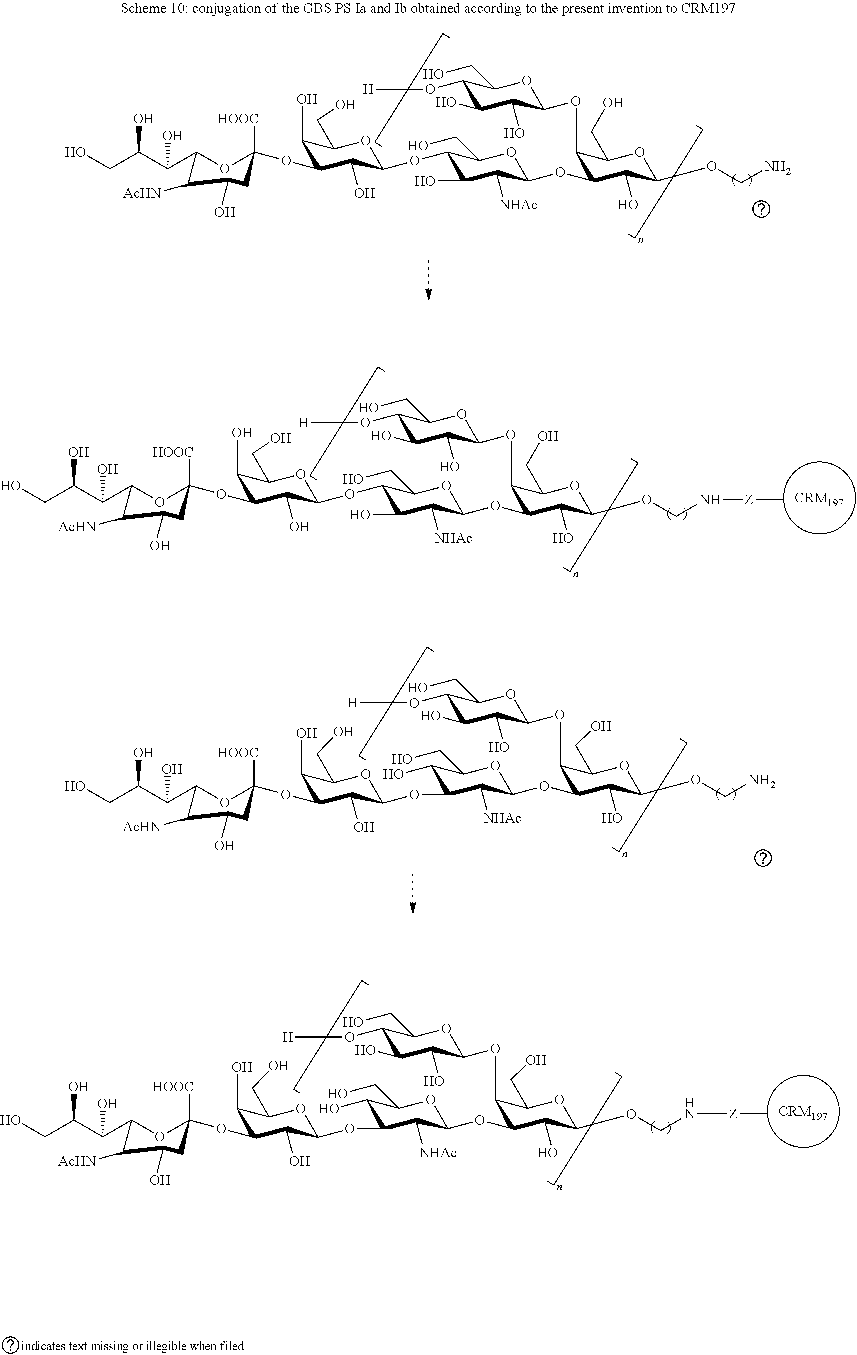

Thus, in a for the embodiment, the invention refers to conjugates of the above identified GBS PS Ia, Ib and III obtained by preparing the GBS PS Ia, Ib and III repeating unit according to the present invention, and connecting the thus obtained building blocks to a carrier protein.

In this direction, in general, covalent conjugation of oligosaccharides to carriers enhances the immunogenicity of oligosaccharides as it converts them from T independent antigens to T-dependent antigens, thus allowing priming for immunological memory. The term “conjugate” refers to an oligosaccharide linked covalently to a carrier protein. In some embodiments an oligosaccharide is directly linked to a carrier protein. In other embodiments an oligosaccharide is indirectly linked to a protein through a spacer or linker. As used herein, the term “directly linked” means that the two entities are connected via a chemical bond, preferably a covalent bond. As used herein, the term “indirectly linked” means that the two entities are connected via a linking moiety (as opposed to a direct covalent bond). In certain embodiments the linker is adipic acid dihydrazide. In other embodiments, the linker is a derivative of a repeating unit. Representative conjugates in accordance with the present invention include those formed by joining together of the oligosaccharide with the carrier protein. Covalent linkage of oligosaccharides to proteins is known in the art and is generally achieved by targeting the amines of lysines, the carboxylic groups of aspartic/glutamic acids or the sulfhydryls of cysteines. For example, cyanate esters randomly formed from sugar hydroxyls can be reacted with the lysines of the protein or the hydrazine of a spacer which are then condensed to the carboxylic acids of the carrier protein via carbodiimide chemistry. Alternatively, aldehydes generated by random periodate oxidation can either be directly used for reductive amination onto the amines of the carrier protein, or converted into amines for following insertion of a spacer enabling the conjugation step to the protein via thioether or amide bond formation. Another strategy employs partial hydrolysis of the purified oligosaccharide and a following fractionation to select population of fragments having a defined average length. A primary amino group can then be introduced at the oligosaccharide reducing termini to be used finally for insertion of either a diester or a bifunctional linker ready for conjugation to the protein.

The term “carrier protein” refers to a protein to which the oligosaccharide is coupled or attached or conjugated, typically for the purpose of enhancing or facilitating detection of the antigen by the immune system. Oligosaccharides are T-independent antigens that are poorly immunogenic and do not lead to long-term protective immune responses. Conjugation of the oligosaccharide antigen to a protein carrier changes the context in which immune effector cells respond to oligosaccharides. The term carrier protein is intended to cover both small peptides and large polypeptides (>10 kDa). The carrier protein may comprise one or more T-helper epitopes.

Useful carrier proteins include bacterial toxins or toxoids, such as diphtheria toxoid or tetanus toxoid. Fragments of toxins or toxoids can also be used e.g. fragment C of tetanus toxoid. The CRM197 mutant of diphtheria toxin [-] is a particularly useful with the invention. Other suitable carrier proteins include the N. meningitidis outer membrane protein, synthetic peptides, heat shock proteins, pertussis proteins, cytokines, lymphokines, hormones, growth factors, human serum albumin (preferably recombinant), artificial proteins comprising multiple human CD4+ T cell epitopes from various pathogen-derived antigens such as N19, protein D from H. influenzae, pneumococcal surface protein PspA, pneumolysin, iron-uptake proteins, toxin A or B from C. difficile, recombinant Pseudomonas aeruginosa exoprotein A (rEPA), a GBS protein, and the like.

Particularly suitable carrier proteins include CRM197, tetanus toxoid (TT), tetanus toxoid fragment C, protein D, non-toxic mutants of tetanus toxin and diphtheria toxoid (DT). Other suitable carrier proteins include protein antigens GBS80, GBS67 and GBS59 from Streptococcus agalactiae and fusion proteins, for example, GBS59(6×D3) disclosed in WO2011/121576 and GBS59(6×D3)-1523 disclosed in EP14179945.2. The use of such GBS protein antigens may be advantageous for a GBS vaccine because, in contrast to heterologous carriers like CRM197, the protein has a dual role increasing immunogenicity of the oligosaccharide whilst also provoking a protective immune response. Hence, the immunological response elicited against the carrier may provide an additional protective immunologic response against GBS, particularly against a GBS protein. In addition, GAS25 from Group A Streptococcus (GAS) could be used to prepare conjugates with immunological activity against GAS/GBS. Another carrier could be genetically modified OMVs (GMMA).

As used herein, the term “glycosylation degree” refers to the number of oligosaccharides per carrier protein molecule and is calculated on the basis of protein and carbohydrate concentration. A loading of between 2 and 9 oligosaccharides per carrier protein molecule has been found to be optimal. It should be understood that such loading values are average values reflecting all of the conjugates in the sample. Alternatively, the glycosylation degree may be described by reference to the oligosaccharide:protein ratio (w/w). For example, a ration between 1:5 (i.e. excess protein) and 10:1 (i.e. excess oligosaccharide).

Compositions may include a small amount of free carrier. When a given carrier protein is present in both free and conjugated form in a composition of the invention, the unconjugated form is preferably no more than 5% of the total amount of the carrier protein in the composition as a whole, and more preferably present at less than 2% by weight.

After conjugation, free and conjugated oligosaccharides can be separated. There are many suitable methods, including hydrophobic chromatography, tangential ultrafiltration, diafiltration etc.

The invention will be now described with the following experimental part, without posing any limitation to its scope.

### Experimental Part

### General Methods

Reactions were monitored by thin-layer chromatography (TLC) on Silica Gel 60 F254 (Sigma Aldrich); after exam under UV light, compounds were visualized by heating with 10% (v/v) ethanolic H2SO4. In the work up procedures, organic solutions were washed with the amounts of the indicated aqueous solutions, then dried with anhydrous Na2SO4, and concentrated under reduced pressure at 30-50° C. on a water bath. Column chromatography was performed on Silica Gel 60 (Sigma Aldrich, 0.040-0.063 nm) or using pre-packed silica cartridges RediSep (Teledyne-Isco, 0.040-0.063 nm) or Biotage SNAP Ultra (Biotage, silica 0.050 nm). Unless otherwise specified, a gradient 0→100% of the elution mixture was applied in a Combiflash Rf (Teledyne-Isco) or Biotage Isolera instrument. Solvent mixtures less polar than those used for TLC were used at the onset of separation. 1H NMR spectra were measured at 400 MHz and 298 K with a Bruker AvanceIII 400 spectrometer; δH values are reported in ppm, relative to internal Me4Si (δH=0.00, CDCl3); solvent peak for D₂O was calibrated at 4.79 ppm. 13C NMR spectra were measured at 100 MHz and 298 K with a Bruker AvanceIII 400 spectrometer; δC values are reported in ppm relative to the signal of CDCl3 (δC=77.0, CDCl3). Assignments of NMR signals were made by homonuclear and heteronuclear 2-dimensional correlation spectroscopy, run with the software supplied with the spectrometer. Assignment of 13C NMR spectra of some compounds was aided by comparison with spectra of related substances reported previously from this laboratory or elsewhere. When reporting assignments of NMR signals, sugar residues in oligosaccharides are indicated with capital letters, uncertain attributions are denoted “/”. Exact masses were measured by electron spray ionization cut-off spectroscopy, using a Q-Tof micro Macromass (Waters) instrument. Structures of these compounds follow unequivocally from the mode of synthesis, NMR data and m/z values found in their mass spectra.

### Syntheses of the Trichloroacetimidate Donors 3 and 4

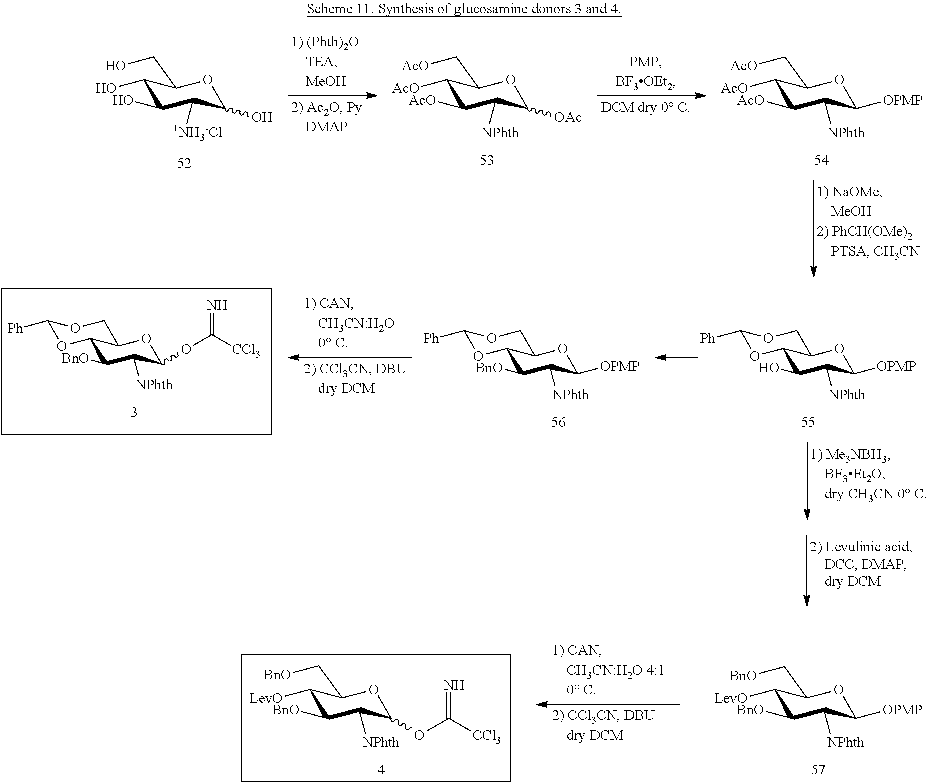

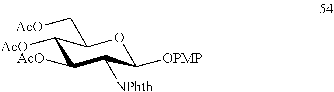

p-Methoxyphenyl 3,4,6-tri-O-acetyl-2-deoxy-2-phthalimido-β-D-glucopyranoside 54. Compound 53 (17 g, 35.6 mmol) was dissolved in dry DCM (60.0 mL) at 0° C. with 4 Å activated molecular sieves (40 g) and stirred for 10 min under nitrogen. Para-methoxyphenol (25 g, 201.4 mmol) and boron trifluoride etherate (24 mL, 194.5 mmol) were added at 0° C. After 1 h the mixture was allowed to warm up to room temperature. Stirring was continued for further 24 h, when TLC showed complete reaction (7:3 Cyclohexane: EtOAc). TEA was added, solid was filtered off and the solvent removed at reduced pressure. The crude was purified by flash chromatography (Cyclohexane:EtOAc) giving 54 (18 g, 89%) quantitative yield as a brown oil. [α]D25=+63.04° (c 1.3, CHCl3). ESI HR-MS m/z [M+Na]+ found 564.1473; calcd 564.1482.

1H NMR (400 MHz, CDCl3) δ 7.80-6.66 (m, 8H, H-Ar), 5.81 (m, 1H, H-1, H-3), 5.19 (t, J=9.7, 1H, H-4), 4.50 (dd, J=8.6, 10.6, 1H, H-2), 4.29 (dd, J=5.3, 12.3 Hz, 1H, H-6a), 4.16 (dd, J=1.9, 12.3, 1H, H-6b), 3.90-3.86 (m, 1H, H-5), 3.66 (s, 3H, OCH3), 2.04, 1.98, 1.82 (3×s, 3H each, 3×CH₃CO).

¹³C NMR (101 MHz, CDCl₃) δ 134.4-114.4 (C-Ar), 97.5 (C-1), 72.0 (C-5), 70.7 (C-3), 68.9 (C-4), 62.0 (C-6), 55.6 (OCH₃), 54.5 (C-2), 20.8, 20.7, 20.5 (3×CH₃CO).

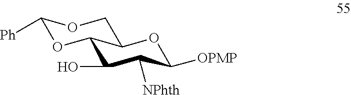

p-Methoxyphenyl 4,6-O-benzylidene-2-deoxy phthalimido-β-D-glucopyranoside 55. Sodium methoxide (until pH 9) was added to a stirred mixture of compound 54 (18.0 g, 35.6 mmol) in methanol (40 mL). After 20 hours the reaction was quenched with Dowex 50WX2. After the filtration of the resin, the filtrate was evaporated under reduced pressure.

To the crude material acetonitrile (30 mL), benzaldehyde dimethyl acetal (6.9 mL, 68 mmol) and para-toluenesulfonic acid (0.470 g, 2.73 mmol) were added. After 3 h the reaction was quenched with triethylamine (4.7 mL), and the mixture was evaporated under reduced pressure.
The crude was purified by flash chromatography (cyclohexane: EtOAc) to afford 55 (6.3 g, 84% yield) as a yellow solid. [α]_{D}²⁵=+64.10° (c 1.2, CHCl₃). ¹H NMR (400 MHz, CDCl₃) δ 7.95-6.77 (m, 13H, H-Ar), 5.84 (d, J=8.5 Hz, 1H, H-1), 5.62 (s, 1H, CHPh), 4.74 (dd, J=8.5, 10.3 Hz, 1H, H-3), 4.54 (dd, J=8.5, 10.7 Hz, 1H, H-2), 4.44 (dd, J=4.5, 10.7 Hz, 1H, H-6), 3.90 (t, J=9.8 Hz, 1H, H-6), 3.81-3.66 (m, 5H, H-4, H-5, OCH₃).

¹³C NMR (101 MHz, CDCl₃) δ 134.4-112.8 (C-Ar), 102.07 (CHPh), 98.10 (C-1), 82.04 (C-4), 68.69 (C-3), 68.63 (C-6), 66.32 (C-5), 56.47 (C-2), 55.70 (OCH₃).

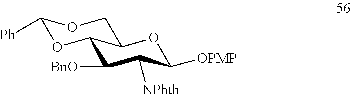

p-methoxyphenyl-3-O-benzyl-4,6-O-benzylidene-2-deoxy-2-phthalimido-β-D-glucopyranoside 55. Sodium hydride (0.148 g, 3.7 mmol) was added to a stirred solution of compound 55 (0.930 g, 1.85 mmol) in N,N-dimethylformamide (7.0 mL) at 0° C. under argon. After 15 min benzyl bromide (0.66 mL, 5.55 mmol) was added, and the mixture was allowed warming to room temperature. After 2 h methanol (10 mL) was added, and the mixture was evaporated under reduced pressure. The product was dissolved in EtOAc and washed with NaHCO₃ (×2), dried (Na₂SO₄) and evaporated under reduced pressure.

The crude was purified by flash chromatography (cyclohexane: EtOAc) to afford 56 (0.900 g, 82% yield) as a yellow solid. [α]_{D}²⁵=+65.17° (c 1.1, CHCl₃). ESI-HR MS m/z [M+Na]⁺ found 616.1866; calcd 616.1947.

¹H NMR (400 MHz, CDCl₃) δ 7.78-6.74 (m, 18H, H-Ar), 5.77 (d, J=7.9 Hz, 1H, H-1), 5.68 (s, 1H, CHPh), 4.86 (d, J=12.4 Hz, 1H, CHHPh), 4.57 (d, J=12.4 Hz, 1H, CHHPh), 4.53-4.48 (m, 2H, H-3, H-2), 4.45 (dd, J=4.9, 10.4 Hz, 1H, H-6), 3.98-3.88 (m, 2H, H-4, H-6), 3.80-3.74 (m, 1H, H-5), 3.73 (s, 3H, OCH₃)

¹³C NMR (101 MHz, CDCl₃) δ 134.00-114.53 (C-Ar), 101.40 (CHPh), 98.00 (H-1), 83.00 (C-4), 74.20 (CH₂Ph), 74.51 (C-3), 68.74 (C-6), 55.74 (C-2), 66.30 (C-5), 55.60 (OCH₃).

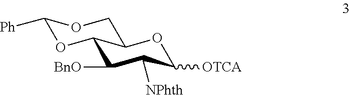

3-O-benzyl-4,6-O-benzylidene-2-deoxy-2-phthalimido-α,β-D-glucopyranoside-trichloroacetimidate 3. Cerium ammonium nitrate (5.110 g, 9.32 mmol) was added to a stirred solution of compound 56 (3 g, 4.66 mmol) in 4:1 acetonitrile: water (50 mL) at 0° C. After 3 h, TLC (7:3 cyclohexane: EtOAc) showed the disappearance of the starting material and the formation of one major spot. The reaction was washed with a solution of NaHCO₃ (×2) and the combined organic phases were dried with Na₂SO₄ and evaporated under reduced pressure. The crude (1.681 g, 3.45 mmol) was dissolved in DCM (10 mL) dry under nitrogen and trichloroacetonitrile (1.730 mL, 17.25 mmol) and 1,8-diazobicyclo[5.4.0]undec-7-ene (0.152 mL, 1.03 mmol) were added. After stirring for 2 h at rt, TLC (7:3 cyclohexane:EtOAc) showed complete reaction. The solvent was removed at reduced pressure and the crude was purified by flash chromatography (cyclohexane:EtOAc) to afford 3 (1.524 g) in 70% yield in 2:1α/β ratio. [α]_{D}²⁵=+64.25° (c 4.15, CHCl₃). ESI MS m/z [M+H]⁺ found 632.06; calcd 631.89.

¹H NMR (400 MHz, CDCl₃) δ 8.5 (s, 1H, NH)7.62-6.80 (m, 14H, H-Ar), 6.42 (d, J=8.4 Hz, H-1_{β}), 6.30 (d, J=3.8 Hz, H-1_{α}), 5.60 (s, 1H, CHPh_{α}), 5.57 (s, 1H, CHPh_{β}), 5.46 (t, H-3_{α}), 4.95 (d, J=11.1 Hz, 1H, CHHPh_{α}), 4.75 (d, J=12.4 Hz, 1H, CHHPh_{β}), 4.62 (d, J=11.1 Hz, 1H, CHHPh_{α}), 4.56 (dd, H-2α), 4.94-4.36 (m, H-2_{β}, H-3_{β}, CHHP_{β}, H-6_{aβ}), 4.31 (dd, H-6_{aα}), 4.18-4.12 (m, H-5_{α}), 3.86-3.76 (m, H-4_{α}, H-6b_{α}, H-4_{β}, H-5_{β}, H-6_{bβ})

¹³C NMR (101 MHz, CDCl₃) δ 134.0-123.4 (C-Ar), 101.4 (CHPh_{β}), 101.3 (CHPh_{α}), 95.4 (C-1_{α}), 94.3 (C-1_{β}), 83.4, 82.5 (C-4), 74.7, 74.3, 74.2 (C-3_{β}), 72.4 (C-3_{α}), 68.5, 66.9 (C-5_{β}), 65.4 (C-5_{α}), 54.7 (C-2).

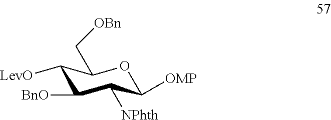

p-methoxyphenyl 3.6-di-O-benzyl-4-O-levulinoyl-2-deoxy-2-phthalimido-β-D-glucopyranoside 41. A solution of 56 (0.500 g, 0.9 mmol) in AcCN (5 mL) was cooled at 0° C. Me₃N BH₃ (0.274 g, 3.76 mmol) and BF₃·O(Et)₂ (0.464 mL, 3.76 mmol) were added, and the reaction was stirred for 2 h under nitrogen. TLC (7:3 cyclohexane:EtOAc) showed complete reaction. TEA was added, until neutral pH, followed by MeOH. The solvent was removed at reduced pressure and the crude was purified by flash chromatography (cyclohexane:EtOAc).

To the obtained product (0.400 g, 0.67 mmol) dissolved in DCM (5 mL), N-N-ethylcarbodiimide hydrochloride (0.206 g, 1.0 mmol), 4-dimethylaminopyridine (0.122 g, 1.0 mmol) and levulynic acid (0.156 g, 1.34 mmol) were added. The mixture was stirred overnight at rt. The solvent was removed by rotary evaporation, and the resulting crude material was purified by flash chromatography (cyclohexane: EtOAc) to give the compound 57 (0.325 g) in 70% yield. [α]_{D}²⁵=+78.3° (c 3.25, CHCl₃). ESI HR-MS m/z [M+Na]⁺ found 716.2447; calcd 716.2472.

¹H NMR (400 MHz, CDCl₃) δ 7.62-6.58 (m, 18H, H-Ar), 5.57 (d, J=8.8 Hz, H-1), 5.14 (t, J=8.9 Hz, 1H, H-4), 4.62 (d, J=11.9 Hz, 1H, CHPh), 4.46-4.42 (m, 3H, CH₂PH, H-3, H-2), 4.28 (d, J=11.9 Hz, 1H, CHHPh), 3.82-3.67 (m, 1H, H-5), 3.61 (s, 3H, OCH₃), 3.58-3.54 (m, 2H, H-6), 2.59 (t, J=6.4 Hz, 2H, CH₂CO), 2.41 (t, J=6.4 Hz, 2H, CH₂COO), 2.07 (s, 3H, CH₃).

¹³C NMR (101 MHz, CDCl₃) δ 137.0-114.0 (C-Ar), 97.4 (C-1), 72.9 (C-4), 74.2 (CH₂Ph), 73.59 (CH₂Ph), 77.24 (C-3), 55.41 (C-2), 73.8 (C-5), 69.56 (C-6), 55.55 (OCH₃), 37.77 (CH₂CO), 29.83 (CH₃), 27.94 (CH₂COO).

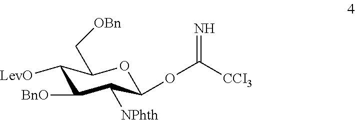

3.6-Di-O-benzyl-4-O-levulinoyl-2-deoxy-2-phthalimido-β-D-glucopyranosyl trichloroacetimidate 4. Cerium ammonium nitrate (0.515 g, 0.94 mmol) was added to a stirred solution of compound 57 (0.325 g, 0.47 mmol) in 4:1 acetonitrile: water (25 mL) at 0° C. After 3 h, a TLC (cyclohexane:ethyl acetate 1:1) showed the disappearance of the starting material and the formation of one major spots. The reaction was washed 2 times with a solution of NaHCO₃ and the organic phase was dried with Na₂SO₄ and evaporated under reduced pressure.

The crude was dissolved in DCM dry (10 mL) under nitrogen and trichloroacetonitrile (0.368 g, 2.55 mmol) and 1,8-diazobicyclo[5.4.0]undec-7-ene (0.023 g, 0.153 mmol) were respectively added. After stirring for 2 h at rt, TLC showed complete reaction (cyclohexane: ethyl acetate 1:1). The solvent was removed at reduced pressure and the crude was purified by flash chromatography (cyclohexane:ethyl acetate) to afford 4 in (0.261 g) 70% yield. NMR was in agreement with literature.

### Syntheses of the Thioglycoside Donors 11 and 2

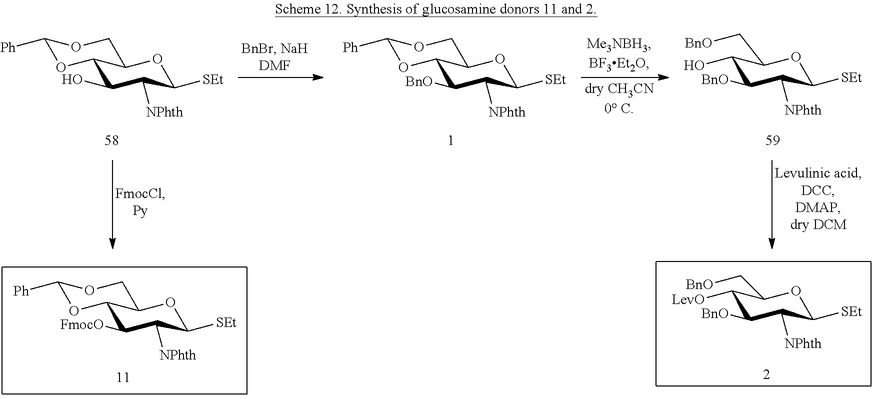

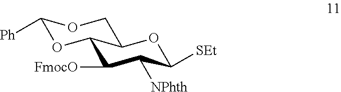

Ethylthiol-4,6-O-benzylidene-2-deoxy-3-O-(fluorenylmethoxy-carbonyl)-2-phthalimido-β-D-glucopyranoside 11. The known compound 58 (0.200 g, 0.45 mmol) was dissolved in dry DCM (10 mL) and Fmoc (0.351 g, 1.36 mmol), Pyridine (0.182 mL, 2.25 mmol) was added at 0° C., and the reaction stirred rt for 1 h. TLC (8:2 cyclohexane:EtOAc) showed complete reaction, the solvent was removed under reduced pressure and the crude was purified by flash chromatography (8: 2 cyclohexane: EtOAc) to afford 11 in 64% yield (0.202 g) as pale yellow oil. [α]_{D}²⁵=+13.53° (c 2.5, CHCl₃). ESI HR-MS (C₃₈H₃₃NO₈S): m/z=[M+Na]⁺ found 686.1807; calcd 686.1825.

¹H NMR (400 MHz, CDCl₃) δ 7.16-7.95 (m, 17H, H-Ar), 5.88 (t, J=9.5 Hz, 1H, H-3), 5.59-5.65 (m, 2H, CHPh, H-1), 4.58 (t, J=10.3 Hz, 1H, H-2), 4.47-4.52 (m, 1H, CH₂ₐ^{Fmoc}), 4.09-4.17 (m, 2H, H-6), 3.92-4.00 (m, 2H, H^{Fmoc}, H-4), 3.84-3.92 (m, 2H, CH_{2b}^{Fmoc}, H-5), 2.65-2.85 (m, 2H, SCH₂), 1.25 (t, J=7.3 Hz, 3H, SCH₂CH₃).

¹³C NMR (101 MHz, CDCl₃) δ 134.4-119.9 (C-Ar), 101.8 (CHPh), 81.9 (C-1), 79.2 (C-4), 74.4 (C-3), 70.5 (C-5), 70.3, 68.6 (C-6), 55.4, 54.1 (C-2), 46.3, 26.9, 24.4 (SCH₂), 14.9 (SCH₂CH₃).

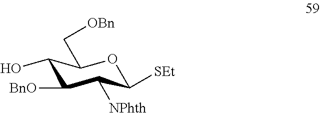

Ethylthio-3,6-di-O-benzyl-2-deoxy-2-phthalimido-13-D-glucopyranoside 59. To a solution of 1(0.500 g, 0.94 mmol) in AcCN (10 mL) was cooled at 0° C. Me₃NBH₃ (3.76 mmol, 0.274 g) and BF₃·OEt₂ (3.76 mmol, 0.464 mL) were added and the reaction was stirred for 2 h under nitrogen. TLC showed complete reaction (7:3 cyclohexane:EtOAc). First TEA and then MeOH were added until neutral pH. The solvent removed at reduced pressure and the crude was purified by flash chromatography (cyclohexane:EtOAc) to afford 59 in 78% yield (0.388 g). NMR spectra were in agreement with those reported in literature. {Barry et al J Am Chem Soc2013, 135, 16895}

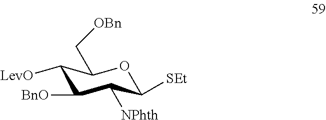

Ethylthio-3,6-di-O-benzyl-2-deoxy-4-O-levulinyl-2-phthalimido-β-D-glucopyranoside 2. Compound 59 (0.388 g, 0.73 mmol) was dissolved in dry DCM (10 mL). LevCl (0.170 g, 1.46 mmol), DCC (0.225 g, 1.09 mmol) and DMAP (0.132 g, 1.09 mmol) were added and the reaction stirred at rt for 3 h. The solvent was removed under reduced pressure, and the crude purified by flash chromatography (cyclohexane: EtOAc) to afford 2 in 77% yield (0.353 g). The NMR data were in agreement with those described in the literature.

### 4.3 Syntheses of Acceptors 5 and 6

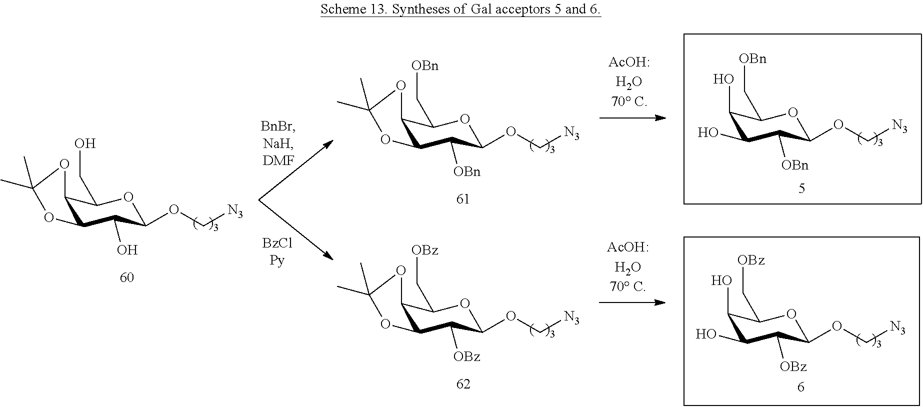

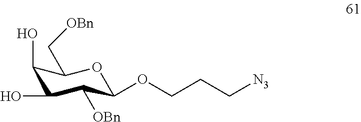

3-Azidopropyl-2,6-di-O-benzyl-β-D-galactopyranoside 5. A suspension of compound 60{Budhadev, 2014 #5477} (3.0 g, 5.7 mmol) in 80% aqueous AcOH (20 mL) was stirred at 70° C. for 2 h when TLC (cyclohexane: EtOAc; 7: 3) showed complete conversion of the starting material to a slower moving spot. Solvents were evaporated in vacuo, coevaporated with toluene to remove traces of AcOH. The residue was purified by flash chromatography using cyclohexane: EtOAc as eluent to give the pure product 61 (2.5 g, 92%) as yellow oil. [α]_{D}²⁵=+12.78° (c 1.05, CHCl₃). ESI HR-MS m/z [M+Na]⁺ found 466.2023; calcd 466.1954.

¹H NMR (400 MHz, CDCl₃) δ 7.46-7.25 (m, 10H, H-Ar), 4.95 (d, J=11.6 Hz, CHHPh), 4.69 (d, J=11.6 Hz, CHHPh), 4.62 (s, 2H, CH₂Ph), 4.38 (d, J=7.7 Hz, 1H, H-1), 4.07-4.00 (m, 2H, OCH_{2b}, H-4), 3.83-3.73 (m, 2H, H-6), 3.69-3.58 (m, 3H, OCH₂ₐ, H-3, H-2), 3.55-3.49 (m, 1H, H-5), 3.44 (t, J=5.4 Hz, 2H, CH₂N₃), 1.93 (m, 2H, CH₂CH₂N₃).

¹³C NMR (101 MHz, CDCl₃) δ 128.6-127.7 (C-Ar), 103.6 (C-1), 79.16 (C-5), 74.73 (CH₂Ph), 73.72 (CH₂Ph), 73.28, 73.13 (C-3), 69.34 (C-6), 68.94 (C-4), 68.51, 66.54 (OCH₂), 48.37 (CH₂N₃), 29.27 (CH₂CH₂N₃).

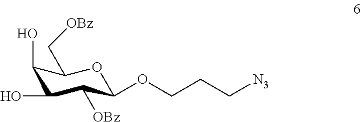

3-Azidopropyl-2,6-di-O-benzoyl-β-D-galactopyranoside 6. A suspension of compound 60 {Budhadev et al Carbohydr. Res. 2014, 394, 26} (3.0 g, 5.7 mmol) in 80% aqueous AcOH (20 mL) was stirred at 70° C. for 2 h when TLC (7:3 cyclohexane: EtOAc) showed complete conversion of the starting material to a slower moving spot. Solvents were evaporated in vacuo, coevaporated with toluene to remove traces of AcOH. The residue was purified by flash chromatography using cyclohexane: EtOAc as eluent to give the pure product 6 (2.5 g, 92%). [α]_{D}²⁵=−3.94° (c 0.45, CHCl₃). ESI HR-MS m/z [M+Na]⁺ found 494.1591; calcd 494.1539.

¹H NMR (400 MHz, CDCl₃) δ 8.06-7.38 (m, 10H, H-Ar), 5.14 (t, J=8.9 Hz, 1H, H-2), 4.69-4.64 (m, 1H, H-6a), 4.56-4.50 (m, 2H, H-1, H-6b), 3.98 (d, J=2.5 Hz, 1H, H-4), 3.97-3.88 (m, 1H, OCH₂a), 3.86-3.83 (m, 1H, H-5), 3.81-3.78 (m, 1H, H-3), 3.59-3.53 (m, 1H, OCH₂b), 3.21 (t, J=6.5 Hz, 2H, CH₂N₃), 1.82-1.65 (m, 2H, CH₂CH₂N3).

¹³C NMR (101 MHz, CDCl₃) δ 167.29, 166.62, 133.7-128.4 (C-Ar), 101.09 (C-1), 99.9, 74.31(C-2), 72.78 (C-3), 72.21 (C-5), 68.59 (C-4), 66.38 (OCH₂), 62.77 (C-6), 47.95 (CH₂N₃), 29.03 (CH₂CH₂N₃).

### Preparations of Disaccharides

Procedure a for Glycosylation with Thioglycoside Donors with NIS/TfOH.

Donor (0.11 mmol) and acceptor (0.1 mmol) with activated 4 Å molecular sieves (0.1 g) were added at the solution of dry DCM (5 mL) and stirred for 20 min under nitrogen. NIS (0.2 mmol) and TfOH (0.02 mmol) were added at −30° C. The reaction was stirred for 2 and then allowed to warm up to room temperature. Stirring was continued for 12 h, monitoring by TLC (Tol:EtOAc or cyclohexane:EtOAc). The reaction was stirred for 12 h monitoring by (Tol:EtOAc or cyclohexane:EtOAc). the reaction was quenched with TEA, the solid filter off and the solvent removed at reduced pressure. The crude was purified by flash chromatography (cyclohexane:EtOAc) to give the purified products.

Procedure B for Glycosylation with Thioglycoside Donors with NIS/AgOTf.

A solution of donor (0.11 mmol) and acceptor (0.1 mmol) with activated 4 Å molecular sieves (0.1 g) in dry DCM (5 mL) was stirred for 20 min under nitrogen. NIS (0.2 mmol) and AgOTf (0.02 mmol) were added at −30° C. The reaction was stirred in the dark allowing to warm up to room temperature. After TLC (Tol:EtOAc or cyclohexane:EtOAc) showed complete reaction, the mixture was quenched with TEA, the solid filter off and the solvent removed at reduced pressure. The crude was purified by flash chromatography (cyclohexane:EtOAc) to give the purified products.

Procedure C for Glycsylation with Trichloroacetimidate Donors

A solution of donor (0.11 mmol) and acceptor (0.1 mmol) with activated 4 Å molecular sieves (0.1 g) in dry DCM (5 mL) was stirred for 20 min under nitrogen. TMSOTf (0.02 mmol) was added at −10° C. After 4 h (TLC; Tol:EtOAc or cyclohexane:EtOAc) the reaction was quenched with TEA, the solid filter off and the solvent removed at reduced pressure. The crude was purified by flash chromatography (Tol:EtOAc or cyclohexane:EtOAc) to afford the purified products.

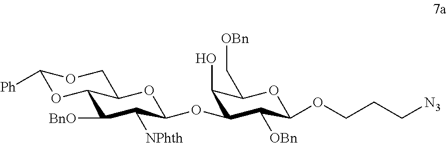

### 3-Azidopropyl-4,6-O-benzilidene-3-O-benzyl-2-deoxy-2-phthalimido-β-D-glucopyranosyl-(1→3)-2,6-di-O-benzyl-β-D-galactopyranoside 7a

Protocol A. After flash chromatography (cyclohexane:EtOAc) a 3:2 mixture (71% yield) of disaccharide 7a and the β-(1→4) product, which could not be isolated as a clean compound, was obtained.

Protocol B 7a, 45% yield.

[α]_{D}²⁵=+14.39° (c 0.25, CHCl₃). ESI HR-MS (C₅₁H₅₂N₄O₁₂): m/z=[M+Na]⁺ found 935.3396; calcd 935.3479.

¹H NMR (400 MHz, CDCl₃) δ 7.55-6.87 (m, 24H, 24H-Ar) 5.65 (s, 1H, CHPh), 5.48 (d, J=12.2 Hz, 1H, H-1^{B}), 4.81 (d, J=12.2 Hz, 1H, CHHPh), 4.59 (s, 2H, CHHPh), 4.50 (d, J=12.2 Hz, 1H, CHHPh), 4.45-4.43 (m, 1H, CHHPh), 4.34-4.32 (m, 2H, H-6^{B}ₐ, H-2^{B}), 4.23-4.20 (m, 2H, CHHPh, H-1^{A}), 4.05 (d, J=2.8 Hz, 1H, H-4^{A}), 3.89-3.78 (m, 4H, H-6^{B}_{b}, OCH₂ₐ, H-6^{A}ₐ, H-5^{B}), 3.74-3.67 (m, 2H, H-6^{A}_{b}, H-3^{B}), 3.60-3.57 (m, 3H, H-4^{B}, H-3^{A}, H-5^{A}), 3.48-3.40 (m, 2H, OCH_{2b}, H-2^{A}), 3.16 (dt, J=3.2, 6.5 Hz, 2H, CH₂N₃), 1.70 (dt, J=6.6, 13.3 Hz, 2H, CH₂CH₂N₃).

¹³C NMR (101 MHz, CDCl₃) δ 129.13-123.28 (C-Ar), 103.34 (C-1^{A}), 101.40 (CHPh), 99.68 (C-1^{B}), 82.86, 82.79 (C-5^{B}), 77.59 (C-2^{A}), 74.44, 74.30 (CH₂Ph), 74.16 (CH₂Ph), 73.67 (CH₂Ph), 69.07 (C-6^{A}), 68.67 (C-6^{B}), 68.19 (C-4^{A}), 66.42 (OCH₂), 66.22 (C-3^{B}), 55.87 (C-2^{B}), 48.12 (CH₂N₃), 29.07 (CH₂CH₂N₃).

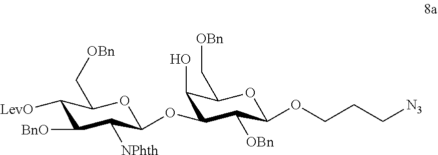

### 3-Azidopropyl 3,6-O-benzyl-2-deoxy-4-O-leyulinoyl-2-phthalimido-β-D-glucopyranosyl-(1→3)-2,6-di-O-benzyl-β-D-galactopyranoside 8a

Protocol A. No reaction observed.

Protocol B. 8a and 8b were obtained in 40% and 27% yield, respectively.

Protocol C. 8a was purified in 31% yield.

3-Azidopropyl-3,6-O-benzyl-2-deoxy-4-O-levulinoyl-2-phthalimido-β-D-glucopyranosyl-(1→3)-2,6-di-O-benzyl-β-D-galactopyranoside (8a). [α]_{D}²⁵=+43.28° (c 0.65, CHCl₃). ESI HR-MS (C₅₆H₆₀N₄O₁₄): m/z=[M+Na]⁺ found 1035.3871; calcd 1035.7878.

¹H NMR (400 MHz, CDCl₃) δ 7.51-6.85 (m, 24H, H-Ar), 5.41 (d, J=8.3 Hz, 1H, H-1^{B}), 5.12 (t, J=9.3 Hz, 1H, H-4^{B}), 4.66 (d, J=12.4 Hz, 1H, CHHPh^{a}), 4.59-4.36 (m, 7H, 5× each CHHPh, H-3^{B}, H-2^{B}), 4.33 (d, J=12.4 Hz, 1H, CHHPh_{b}), 4.23 (d, J=11.1 Hz, 1H, CHHPh), 4.20 (d, J=7.5 Hz, 1H, H-1^{A}), 4.08 (d, J=2.9 Hz, 1H, H-4^{A}), 3.90-3.80 (m, 1H, OCH₂ₐ), 3.73-3.42 (m, 9H, H-5^{B}, H-6^{B}_{a,b}, H-6^{A}_{a,b}, H-2,5,3^{A}, OCH₂b) 3.18 (t, J=6.9 Hz, CH₂N₃), 2.71-2.68 (m, 2H, CH₂^{Lev}), 2.59-2.43 (m, 2H, CH₂^{Lev}), 2.18 (s, 3H, CH₃^{Lev}) 1.76-1.67 (m, 2H, CH₂CH₂N₃).

¹³C NMR (101 MHz, CDCl₃) δ 128.42-123.29 (C-Ar), 103.15 (C-1^{A}), 98.59 (C-1B), 83.55 (C-3^{A}), 77.5 (C-5^{A}), 76.9 (C-3^{B}), 74.47 (CH₂Ph), 74.10 (CH₂Ph), 74.16 (CH₂Ph), 73.57 (CH₂Ph), 73.45 (C-5^{B}), 73.18 (C-2^{A}), 72.45 (C-4^{B}), 69.66 (C-6^{B}), 69.46 (C-6^{A}), 67.84 (C-4^{A}), 66.32 (OCH₂), 55.42 (C-2^{B}), 48.17 (CH₂N₃), 37.70 (cH₂^{Lev}), 29.79 (cH₃^{Lev}), 29.10 (CH₂CH₂N₃), 27.88 (CH₂^{Lev}).

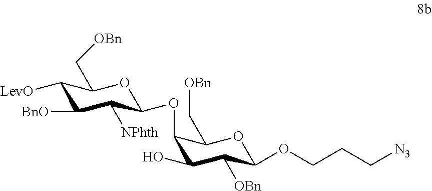

3-Azidopropyl-3,6-O-benzyl-2-deoxy-4-O-levulinoyl-2-phthalimido-β-D-glucopyranosyl-(1→4)-2,6-di-O-benzyl-β-D-galactopyranoside (8b). [α]_{D}²⁵=+18.87° (c 1.9, CHCl₃). ESI HR-MS (C₅₆H₆₀N₄O₁₄): m/z=[M+Na]⁺ found 1035.3914; calcd 1035.3878.

¹H NMR (400 MHz, CDCl₃) δ) δ 7.23-6.83 (m, 24H, H-Ar), 5.23 (d, J=8.4 Hz, 1H, H-1^{B}), 5.09 (t, J=9.7 Hz, 1H, H-4^{B}), 4.59 (d, J=8.5 Hz, 1H, CHHPh), 4.54-4.22 (m, 9H, 7×each CHHPh, H-3^{B}, H-2^{B}), 4.07 (d, J=7.7 Hz, 1H, H-1^{A}), 3.84 (d, J=2.7 Hz, 1H, H-4^{A}), 3.80-3.42 (m, 8H, OCH_{2a,b}, H-6^{B}_{a,b}, H-6^{A}_{a,b}, H-5^{A}, H-5^{B}), 3.32 (dd, J=2.8, 9.7 Hz, 1H, H-3^{A}), 3.24 (t, J=6.8 Hz, 2H, CH₂N₃), 2.87 (t, J=8.6 Hz, 1H, H-2^{A}), 2.54 (t, J=6.7 Hz, CH₂^{Lev}), 2.35 (t, J=6.7 Hz, 1H, CH₂^{Lev}), 2.08 (s, 1H, CH₃^{Lev}), 1.80-1.67 (m, 2H, CH₂CH₂N₃).

¹³C NMR (101 MHz, CDCl₃) δ 133.6-122.9 (C-Ar), 103.08 (C-1^{A}), 99.70 (C-1B), 79.89 (C-2^{A}), 76.8 (C-3B), 76.6 (C-4^{A}), 73.79 (C-5^{A}), 73.46 (CH₂Ph), 73.34 (2×each CH₂Ph), 72.86 (CH₂Ph), 72.70 (C-3^{A}), 72.64 (C-5^{B}), 69.84 (C-6^{A,B}), 69.71 (C-6^{A,B}), 66.02 (OCH₂), 55.68 (C-2^{B}), 48.35 (CH₂N₃), 37.74 (CH₂^{Lev}), 29.80 (CH₂^{Lev}), 29.17 (CH₂CH₂N₃), 27.95 (CH₃^{Lev}).

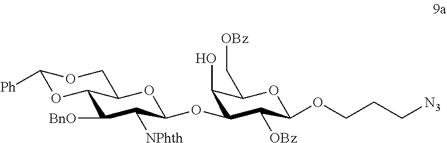

### 3-Azidopropyl-4,6-O-benzilidene-3-O-benzyl-2-deoxy-2-phthalimido-β-D-glucopyranosyl-(1→3)-2,6-di-O-benzoyl-β-D-galactopyranoside 9a

Procotol A. 9a, 53% yield.

Protocol B. 9a, 64% Yield.

[α]_{D}²⁵=+44.98° (c 0.4, CHCl₃). ESI HR-MS (C₅₁H₄₈N₄O₁₄): m/z=[M+Na]⁺ found 963.3200; calcd 963.3065.

¹H NMR (400 MHz, CDCl₃) δ 8.08-6.81 (m, 24H, H-Ar), 5.63 (s, 1H, CHPh), 5.41 (d, J=8.2 Hz, 1H, H-1^{B}), 5.32 (t, J=8.9 Hz, 1H, H-2^{A}), 4.74-4.60 (m, 3H, 2×each H-6^{A}, CHHPh), 4.43 (d, J=12.2 Hz, 1H, CHHPh), 4.40 (d, J=8.1 Hz, 1H, H-1^{A}), 4.37-4.33 (m, 2H, H-6^{B}ₐ, H-3^{B}), 4.27 (t, J=9.2 Hz, 1H, H-2^{B}), 4.21 (d, J=2.8 Hz, 1H, H-4^{A}), 3.92-3.80 (m, 5H, H-5^{A}, H-3^{A}, H-4^{B}, H-6^{B}_{b}, OCH₂ₐ), 3.70-3.64 (m, 1H, H-5^{B}), 3.42 (dt, J=4.3, 8.6 Hz, 1H, OCH_{2b}), 3.09-3.0 (m, 2H, CH₂N₃), 1.68-1.56 (m, 2H, CH₂CH₂N₃).

¹³C NMR (101 MHz, CDCl₃) δ 166.4-164.5 (2×C=O), 137.7-122.7 (C-Ar), 101.4 (CHPh), 101.2 (C-1^{A}), 99.9 (C-1^{B}), 82.7 (C-4^{B}), 80.8 (C-3^{A}), 74.2 (C-3^{B}), 74.0 (CH₂Ph), 71.9 (C-5^{A}), 70.5 (C-2^{A}), 68.6 (C-6^{B}), 68.5 (C-4^{A}), 66.3 (C-5^{B}), 65.9 (OCH₂), 63.5 (C-6^{A}), 55.5 (C-2^{B}), 47.8 (CH₂N₃), 28.9 (CH₂CH₂N₃).

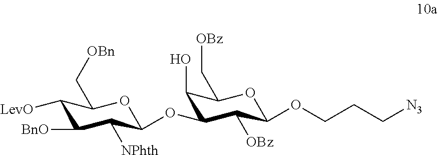

### 3-Azidopropyl-3,6-O-benzyl-2-deoxy-4-O-levulinoyl-2-phthalimido-β-D-glucopyranosyl-(1→3)-2,6-di-O-benzoyl-β-D-galactopyranoside

Protocol A. No product formation.

Protocol B. 10a, 63% yield.

Protocol C. 10a, 33% yield.

[α]_{D}²⁵=+62.78° (c 1.4, CHCl₃). ESI HR-MS (C₅₆H₅₆N₄O₁₆): m/z=[M+Na]⁺ found 1063.3577; calcd 1063.3589.

¹H NMR (400 MHz, CDCl₃) δ 8.05-6.84 (m, 24H, H-Ar), 5.36 (d, J=8.3 Hz, 1H, H-1^{B}), 5.31 (d, J=8.9 Hz, 1H, H-4^{B}), 5.08 (t, J=9.1 Hz, 1H, H-2^{A}), 4.61-4.44 (m, 5H, H-6^{A}_{a,b}, 3×each CHHPh), 4.39 (d, J=8.0 Hz, 1H, H-1^{A}), 4.36-4.34 (m, 1H, H-3^{B}), 4.31-4.29 (m, 1H, H-2^{B}), 4.27-4.24 (m, 2H, H-4^{A}, CHHPh), 3.86-3.79 (m, 4H, OCH₂ₐ, H-3,5^{A}, H-5^{B}), 3.60-3.58 (m, 2H, H-6^{B}), 3.41 (dt, J=4.5, 9.2 Hz, 1H, OCH_{2b}), 3.20 (t, J=6.5 Hz, 2H, CH₂N₃), 3.1-2.9 (m, 2H, CH₂^{Lev}), 2.7-2.65 (m, 2H, CH₂^{Lev}), 2.16 (s, 3H, CH₃^{Lev}), 1.64-1.56 (m, 2H, CH₂CH₂N₃).

¹³C NMR (101 MHz, CDCl₃) δ 166.8, 164.0 (C═O), 133.1-127.3 (C-Ar), 101.13 (C-1^{A}), 101.40, 98.82 (C-1^{B}), 81.05, 73.91, 73.49, 72.33, 72.04 (C-2^{A}), 70.49 (C-4^{B}), 69.52, 68.07, 65.84 (OCH₂), 63.73, 55.10 (C-2B), 47.79 (CH₂N₃/CH₂^{Lev}), 37.68 (cH₂^{Lev}), 29.78 (CH₃^{Lev}), 28.86 (CH₂CH₂N₃), 27.84 (CH₂^{Lev}).

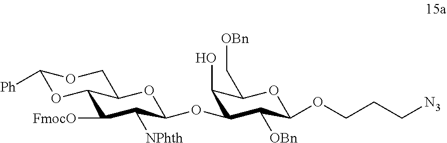

### 3-Azidopropyl-4,6-O-benzylidene-2-deoxy-2-phthalimido-3-O-(9-fluorenylmethyloxycarbonyl)-β-D-glucopyranosyl-(1→3)-2,6-di-O-benzyl-β-D-galactopyranoside 15a

Protocol A. After flash chromatography (Tol:EtOAc) 15a and 15b were purified in 30% and 10% yield, respectively.

Protocol B. 15a, 38% yield; 15b, 26% yield.

3-Azidopropyl-4,6-O-benzylidene-2-deoxy-2-phthalimido-3-O-(9-fluorenylmethyloxycarbonyl)-β-D-glucopyranosyl-(1→3)-2,6-di-O-benzyl-β-D-galactopyranoside (26). [α]_{D}²⁵=+10.37° (c 0.9, CHCl₃). ESI HR-MS (C₅₉H₅₆N₄O₁₄): m/z=[M+Na]⁺ found 1067.3629; calcd 1067.3691.

¹H NMR (400 MHz, CDCl₃) δ 7.63-6.85 (m, 27H, H-Ar), 5.71 (t, J=10.1 Hz, 1H, H-3^{B}), 5.62 (d, J=8.7 Hz, 1H, H-1^{B}), 5.51 (s, 1H, CHPh), 4.51, 4.48 (2 d, J=12.3 Hz, 1H each, CHHPh), 4.47 (dd, J=8.3, 10.4 Hz, 1H, H-2B), 4.38 (d, J=11.7 Hz, 1H, CHHPh), 4.31 (dd, J=4.6, 10.2 Hz, 1H, H-6^{A}), 4.17-4.13 (m, 2H, H-1^{A}, CHHPh), 4.01-3.99 (m, 2H, H-4^{A}, CH₂^{Fmoc}), 3.87-3.81 (m, 2H, H-4B, CH^{Fmoc}), 3.79-3.61 (m, 5H, H-6^{B}_{a,b}, H-6^{A}_{b}, OCH₂ₐ, H-5B), 3.57 (dd, J=3.3, 9.5 Hz, 1H, H-3^{A}), 3.51 (t, J=6.0 Hz, 1H, H-2^{A}), 3.39 (dd, J=7.7, 9.2 Hz, 1H, H-5^{A}), 3.36-3.33 (m, 1H, OCH_{2b}), 3.06 (dt, J=3.5, 6.81 Hz, 2H, CH₂N₃), 1.64-1.57 (m, 2H, CH₂CH₂N₃).

¹³C NMR (101 MHz, CDCl₃) δ 134.04-119.88 (C-Ar), 103.42 (C-1^{A}), 101.83 (CHPh), 99.45 (C-1^{B}), 83.06 (C-3^{A}), 78.91 (C-4^{B}), 77.55 (C-5^{A}), 74.27 (CH₂Ph), 73.69 (C-3^{B}), 73.49 (CH₂Ph), 73.38, 72.73 (C-2^{A}), 70.36, 69.02 (C-6B), 68.57 (C-6^{A}), 68.20 (C-4^{A}), 66.48 (OCHH), 66.29 (C-5^{B}), 60.42, 55.25 (C-2^{B}), 48.36, 48.12 (CH₂N₃), 46.32, 29.08 (CH₂CH₂N₃), 28.25, 21.07, 14.21.

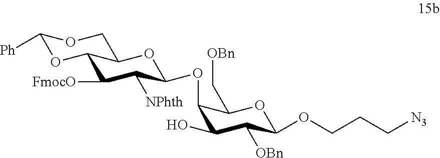

### 3-Azidopropyl-4,6-O-benzylidene-2-deoxy phthalimido-3-O-(9-fluorenylmethyloxycarbonyl)-β-D-glucopyranosyl-(1→4)-2,6-di-O-benzyl-β-D-galactopyranoside (15b)

[α]_{D}²⁵=−8.99° (c 0.85, CHCl₃). ESI HR-MS (C₅₉H₅₆N₄O₁₄): m/z=[M+Na]⁺ found 1067.3680; calcd 1067.3691.

¹H NMR (400 MHz, CDCl₃) δ 7.70-6.94 (m, 27H, H-Ar), 5.89 (t, J=9.3 Hz, 1H, H-3B), 5.51 (s, 1H, CHPh), 5.47 (d, J=7.8 Hz, 1H, H-1^{B}), 4.55-4.48 (m, 3H, 2 CHHPh, H-2^{B}), 4.16-4.08 (m, 3H, 2 CHHPh, includ. d, 4.12, J=7.7 Hz, H-1^{A}), 3.95 (m, 2H, H^{Fmoc}, H-4^{A}), 3.89-3.62 (m, 7H, includ. H-4,5,6^{B}, H-6^{A}, OCH₂ₐ), 3.58 (m, 1H, OCH_{2b}), 3.47 (m, 1H, H-5^{A}), 3.36 (dd, J=2.9, 7.2 Hz, 1H, H-3^{A}), 3.30 (m, 2H, CH₂N₃), 3.00 (dd, J=7.7, 9.8 Hz, 1H, H-2^{A}), 1.80 (m, 2H, CH₂CH₂N₃).

¹³C NMR (101 MHz, CDCl₃) δ 134.0-119.9 (C-Ar), 103.27 (C-1^{A}), 101.7 (PhCH), 100.4 (C-1^{B}), 79.9 (C-2^{A}), 79.1 (C-4^{B}), 77.2 (C-4^{A}), 74.9 (PhCH₂), 73.6 (C-3^{B}), 73.4 (PhCH₂), 72.8 (C-3^{A}), 70.2 (C-5^{A/B}), 68.8, 68.6 (C-6^{A,B}), 66.2 (OCH₂), 65.4 (C-5^{A/B}), 55.3 (C-2^{B}), 48.4 (CH₂N₃), 46.4 (CH^{Fmoc}) 29.2 (CH₂CH₂N₃).

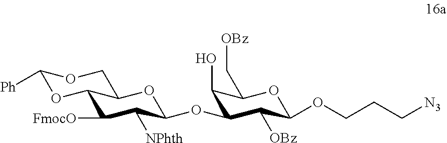

### 3-Azidopropyl-4,6-O-benzylidene-2-deoxy-2-phthalimido-3-O-(9-fluorenylmethyloxycarbonyl)-β-D-glucopyranosyl-(1→3)-2,6-di-O-benzoyl-β-D-galactopyranoside 16a

Protocol A. 16a, 40% yield.

Protocol B. 16a, 38% yield.

Protocol C. 16a, 63% yield.

[α]_{D}²⁵=+36.44° (c 0.65, CHCl₃). ESI HR-MS (C₅₉H₅₂N₄O₁₆): m/z=([M+Na]⁺ found 1095.3247; calcd 1095.3276.

¹H NMR (400 MHz, CDCl₃) δ 8.01-7.07 (m, 27H, H-Ar), 5.62-5.57 (m, 1H, H-3^{B}), 5.56 (d, J=8.5 Hz, 1H, H-1^{B}), 5.50 (s, 1H, CHPh), 5.27 (t, J=9.1 Hz, 1H, H-2^{A}), 4.63 (dd, J=11.0, 5.0 Hz, 1H, H-6^{A}ₐ), 4.55 (dd, J=11.0, 5.0 Hz, 1H, H-6^{A}_{b}), 4.41 (t, J=9.4 Hz, 1H, H-2^{B}), 4.34 (d, J=8.0 Hz, 1H, H-1^{A}), 4.29 (dd, J=4.5, 9.8 Hz, 1H, H-6Bₐ), 4.15 (d, J=3.2 Hz, 1H, H-4^{A}), 3.93 (d, J=7.8 Hz, 2H, CH₂^{Fmoc}), 3.85-3.59 (m, 7H, H-5,3^{A}, H-4,5^{B}, H-6^{B}_{b}, CH^{Fmoc}, OCH₂ₐ), 3.36-3.30 (m, 1H, OCH_{2b}), 3.01-2.87 (m, 2H, CH₂N₃), 1.63-1.43 (m, 2H, CH₂CH₂N₃).

¹³C NMR (101 MHz, CDCl₃) δ 133.78-119.84 (C-Ar), 101.84 (CHPh), 101.24 (C-1^{A}), 99.67 (C-1^{B}), 81.09 (C-4^{B}), 78.77 (C-3^{A}), 73.23, 71.88 (C-5^{A}), 70.48)(CH₂^{Fmoc}), 70.33 (C-2^{B}), 68.52 (C-4^{A}), 68.47 (C-6^{B}), 66.34 (C-5^{B}), 65.99 (OCH₂), 63.38 (C-6^{A}), 54.90 (C-2^{B}), 47.76 (CH₂N₃), 46.25)(CH^{Fmoc}), 28.86 (CH₂CH₂N₃).

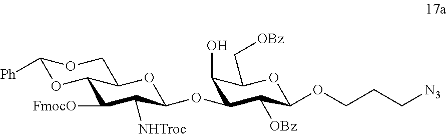

### 3-Azidopropyl-{4,6-O-benzylidene 3-O-(9H-fluoren-9-ylmethyl carbonate)-2-deoxy-2-[[(2,2,2-trichloroethoxy)carbonyl]amino]-β-D-glucopyranosyl-(1→3)}-2,6-di-O-benzoyl-β-D-galactopyranoside 17a

Protocol B. 17a, 65% yield.

Protocol C. 17a, 70% yield.

[α]_{D}²⁵=−10.29° (c 0.55, CHCl₃). ESI HR-MS (C₅₄H₅₁Cl₃N₄O₁₆): m/z=([M+Na]⁺ found 1134.2173; calcd (1134.2138).

¹H NMR (400 MHz, CDCl₃) δ 8.17-7.10 (m, 23H, Ar—H), 5.56-5.48 (m, 2H, CHPh, H-2^{A}), 5.24 (t, 1H, J=10.0 Hz, H-3B), 5.09 (d, 1H, J=8.25 Hz, NH), 4.99 (d, 1H, J=7.8 Hz, H-1B), 4.73 (dd, 1H, J=11.39 Hz, J=4.94 Hz, CHHCCl₃), 4.65 (dd, 1H, J=11.39 Hz, J=7.14 Hz, CHHCCl₃), 4.55 (d, 1H, J=8.0 Hz, H-1^{A}), 4.37-4.25 (m, 4H, incl. CH₂^{Fmoc}, H-6^{A}ₐ, H-6^{B}ₐ), 4.25-4.15 (m, 2H, CH^{Fmoc}, H-5^{B}), 4.09 (d, 1H, J=12.0 Hz, H-6^{A}_{b}), 4.03-3.91 (m, 3H, incl. OCHH, H-3^{A}, H-4^{A}), 3.86-3.70 (m, 3H, H-2^{B}, H-6^{B}_{b}, H-4^{B}), 3.69-3.48 (m, 2H, H-5^{A}, OCHH), 3.26-3.13 (m, 2H, CH₂N₃), 2.05-1.51 (m, 2H, CH₂CH₂N₃).

¹³C NMR (101 MHz, CDCl₃) δ 166.42, 164.95 (CO esters), 138.2-115.4 (m, 26C, C-Ar), 101.65 (CHPh), 101.56 (C-1^{A}), 101.20 (C-1^{B}), 80.43 (C-4^{A}), 78.45 (C-4^{B}), 74.22 (C-3^{B}), 73.77 (C-6^{A}), 72.10 (C-3^{A}), 70.68 (C-2^{A}), 70.37)(CH₂^{Fmoc}), 68.81 (C-5^{B}), 68.39 (C-6^{B}), 66.33 (OCH₂), 63.15 (CH₂CCl₃), 57.24 (C-5^{A}), 55.99 (C-2^{B}), 47.78 (CH₂N₃), 46.77)(CH^{Fmoc}), 29.6 (CH₂CH₂N₃).

### Syntheses of Trisaccharides

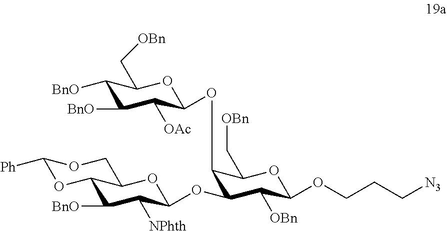

3-Azidopropyl-[2-O-acetyl-3,4,6-tri-O-benzyl-β-D-glucopyranosyl)-(1→4)]-{4,6-O-benzilidene-3-O-benzyl-2-deoxy-2-phthalimido-β-D-glucopyranosyl-(1→3)}-2,6-di-O-benzyl-β-D-galactopyranoside 19a. A solution of donor 18 (0.050 g, 0.08 mmol) and disaccharide acceptor 7a (0.061 g, 0.067) with activated 4 Å molecular sieves (0.100 g) in dry DCM (4 mL) was stirred for 20 min under nitrogen. TMSOTf (3 μL, 0.013 mmol) was added at −10° C. After 4 h (TLC; 9:1 Tol: EtOAc) the reaction was quenched with TEA, the solid filter off and the solvent removed under pressure. The crude was purified by flash chromatography (Tol:EtOAc) to afford the thrisaccharide 33 in 73% yield (0.068 g) as a pale yellow solid.

[α]_{D}²⁵=+24.86° (c 0.8, CHCl₃). ESI HR-MS (C₈₀H₈₂N₄O₁₈): m/z=[M+Na]⁺ found 1409.5419; calcd 1409.5522.

¹H NMR (400 MHz, CDCl₃) δ 7.55-6.85 (m, 39H, H-Ar), 5.66 (s, 1H, CHPh), 5.56 (d, J=8.8 Hz, 1H, H-1^{B}), 5.00-4.98 (m, 2H, H-1,2^{C}), 4.91-4.81 (m, 3H, 3 CHHPh), 4.60 (d, J=10.3 Hz, 1H, CHHPh), 4.53-4.36 (m, 8H, 7 CHHPh, H-6ₐ^{C}), 4.28 (t, J=9.20 Hz, 1H, H-2^{B}), 4.19 (d, J=7.6 Hz, 1H, H-1^{A}), 4.16 (d, J=2.3 Hz, 1H, H-4^{A}), 4.11 (d, J=11.5 Hz, 1H, CHHPh), 3.94-3.89 (m, 1H, H-3^{C}), 3.86-3.49 (m, 13H, H-3^{A,B}, H-4^{B,C}, H-5^{A,C}, H-6_{b}^{B}, OCH₂ₐ), 3.42-3.37 (m, 1H, OCH_{2b}), 3.30 (t, J=8.8 Hz, 1H, H-2^{A}), 3.13-3.10 (m, 2H, CH₂N₃), 1.79 (s, 3H, CH₃CO), 1.73-1.62 (m, 2H, CH₂CH₂N₃).

¹³C NMR (101 MHz, CDCl₃) δ 169.54 (C═O), 133.72-123.05 (C-Ar), 103.48 (C-1^{A}), 101.31 (PhCH), 100.32 (C-1^{C}), 100.21 (C-1^{B}), 83.4 (C-3^{C}), 83.1, 81.2, 78.6 (C-2^{A}), 77.8, 75.2, 75.0, 74.9 (3 PhCH₂), 74.5, 74.4 (C-4^{A}), 74.1, 73.7, 73.5, 73.4, 73.1 (3 PhCH₂), 69.8, 69.1, 68.7 (C-6^{A-C}), 66.3 (OCH₂), 65.91, 56.3 (C-2^{B}), 48.2 (CH₂N₃), 29.1 (CH₂CH₂N₃), 20.8 (CH₃CO).

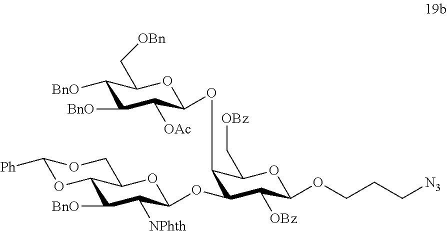

### 3-Azidopropyl-[(2-O-acetyl-3,4,6-tri-O-benzyl-β-D-glucopyranosyl)-(1→4)]-{4,6 benzilidene-3-O-benzyl-2-deoxy-2-phthalimido-β-D-glucopyranosyl-(1→3)}-2,6-di-O-benzoyl+D-galactopyranoside 19b

A solution of donor 18 (0.048 g, 0.077 mmol) and disaccharide acceptor 9a (0.060 g, 0.064 mmol) with activated 4 Å molecular sieves (0.100 g) in dry DCM (4 mL) was stirred for 20 min under nitrogen. TMSOTf (2 μL, 0.013 mmol) was added at −10° C. After 4 h (TLC; Tol: EtOAc 9: 1) the reaction was quenched with TEA, the solid filter off and the solvent removed under pressure. The crude was purified by flash chromatography (Tol: EtOAc) to afford the thrisaccharide 34 in 65% yield (0.058 g) as a pale yellow solid.

[α]_{D}²⁵=+18.18° (c 1.45, CHCl₃). ESI HR-MS (C₈₀H₇₈N₄O₂₀): m/z=[M+Na]⁺ found 1437.5027; calcd 1437.5107.

¹H NMR (400 MHz, CDCl₃) δ 8.08-6.80 (m, 39H, H-Ar), 5.66 (s, 1H, CHPh), 5.43 (d, J=8.4 Hz, 1H, H-1^{B}), 5.15 (t, J=8.3 Hz, 1H, H-2^{A}), 5.03 (t, J=7.4 Hz, 1H, H-2^{C}), 4.99 (d, J=8.4 Hz, 1H, H-1^{C}), 4.91 (br. s, 2H, 2 CHHPh), 4.86 (d, J=10.7 Hz, CHHPh), 4.75 (d, J=10.7 Hz, CHHPh), 4.71 (d, J=4.5, 12.3 Hz, H-6ₐ^{A}), 4.62-4.37 (m, 6H, 4 CHHPh, H-6_{b}^{A}, H-6ₐ^{B}), 4.35 (d, J=2.7 Hz, H-4^{A}), 4.32 (t, J=8.5 Hz, H-3^{B}), 4.22 (t, J=9.3 Hz, 1H, H-2^{B}), 3.94 (t, J=9.5 Hz, 1H, H-3^{C}), 3.90-3.61 (m, 9H, H-3^{A}, H-6_{b}^{B}, H-6^{C}, OCH₂ₐ), 3.55-3.52 (m, 1H, H-5^{C}), 3.44-3.39 (m, 1H, OCH_{2b}), 3.08-2.96 (m, 2H, CH₂CH₂N₃), 2.02 (s, 3H, CH₃CO), 1.71-1.55 (m, 2H, CH₂CH₂N₃).

¹³C NMR (101 MHz, CDCl₃) δ 171.2, 166.4, 164.7 (3 CO), 133.5-122.9 (C-Ar), 101.3 (PhCH), 101.1 (C-1^{A}), 100.4 (C-1^{C}), 100.3 (C-1^{B}), 83.4 (C-3^{C}), 83.0 (C-4^{B}), 80.0 (C-3^{A}), 78.0, 75.5 (C-5^{C}), 75.4, 75.3, 74.3 (3 PhCH₂), 74.2 (C-3^{B}), 74.0 (C-4^{A}), 73.5 (PhCH₂), 73.3 (C-2^{C}), 72.2, 70.6 (C-2^{A}), 69.2, 68.7 (C-6^{A,B}), 66.1 (C-5^{A}), 65.3 (OCH₂), 64.4 (C-6^{C}), 55.9 (C-2^{B}), 47.9 (CH₂N₃), 28.9 (CH₂CH₂N₃), 20.7 (CH₃CO).

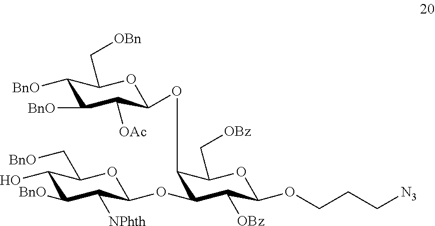

### 3-Azidopropyl-[(2-O-acetyl-3,4,6-tri-O-bensyl-β-D-glucopyranosyl)-(1→4)]-{3,6-di-O-benzyl-2-deoxy-2-phthalimido-β-D-glucopyranosyl-(1→3)}-2,6-di-O-benzoyl-13-D-galactopyranoside 20

A solution of trisaccharide 19b (0.258 g, 0.181 mmol) acetonitrile was cooled down to 0° C. Trimethylamino borane complex (0.053 g, 0.72 mmol) was added, followed by BF₃·Et₂O (0.090 mL, 0.72 mmol). The reaction mixture was stirred at 0° C. for 3 h. Analytical TLC which (Tol/EtOAc 8.2) showed formation of a new spot with lower R_{f}. The reaction was quenched by addition of Et₃N and MeOH, then evaporated under vacuum. The crude was purified by column chromatography (Tol/EtOAc). Clean fractions were collected and evaporated to dryness affording trisaccharide 20 (0.180 g, 70% yield) as a colorless oil.

[α]_{D}²⁵=+204.32 (c 0.39, CHCl³). ESI HR-MS (C₈₀H₈₀N₄O₂₀): m/z=([M+Na]⁺ found 1439.5051; calcd (1439.5264)

¹H NMR (400 MHz, CDCl₃) δ 8.15-6.54 (m, 39H, H-Ar), 5.29 (d, 1H, J=7.4 Hz, H-1^{B}), 5.08 (t, 1H, J=9.1 Hz, H-2^{A}), 5.18-4.9 (m, 2H, incl. H-1^{C}, H-2^{C}), 4.81 (s, 2H, 6-OCH₂Ph^{B}), 4.76 (d, 1H, J=11.1), 4.66-4.56 (m, 2H, incl. H-6^{A}ₐ), 4.56-4.33 (m, 7H, incl. H-6^{A}_{b}), 4.32-4.26 (m, 2H, incl. H-1^{A}, H-4^{A}), 4.13-4.01 (m, 2H, incl. H-2^{B}, H-3^{B}), 3.87-3.66 (m, 6H, incl. H-3^{C}, H-3^{A}, H-5^{A}, H-4^{B}, H-6^{B}_{a,b}, OCHHN₃), 3.66-3.52 (m, 4H, incl. H-4^{C}, H-5^{B}, H-6^{C}_{a,b}), 3.49-3.40 (m, 1H, H-5^{C}), 3.40-3.29 (m, 1H, —OCHH), 3.02-2.85 (m, 2H, —CH₂CH₂N₃), 1.87 (s, 3H, CH₃CO), 1.66-1.42 (m, 2H, CH₂N₃).

¹³C NMR (101 MHz, CDCl₃) δ 170.5, 166.4, 164.5 (3×CO esters), 138.7-122.9 (m, 43C, C-Ar), 101.0 (C-1^{A}), 100.5 (C-1^{C}), 99.7 (C-1^{B}), 83.4 (C-3^{C}), 79.7 (C-3^{A}), 78.5 (C-3^{B}), 77.9 (C-4^{C}), 75.3 (C-5^{C}), 75.2, 75.0, 74.6 (3×CH₂Ph), 74.3 (C-4^{A}), 74.2 (C-5^{A}), 73.7 (C-5^{B}), 73.7, 73.5 (2×CH₂Ph), 73.1 (C-2^{C}), 72.3 (C-4^{B}), 70.7 (C-6^{B}), 70.6 (C-2^{A}), 69.2 (C-6c), 65.1 (OCH₂), 64.8 (C-6^{A}), 55.5 (C-2^{B}), 47.9 (CH₂N₃), 28.9 (CH₃), 20.6 (CH₂CH₂N₃).

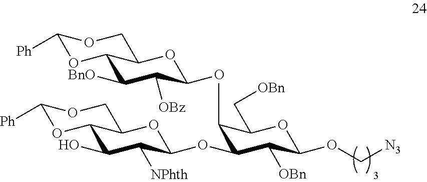

### 3-Azidopropyl [4,6-O-benziliden-2-O-benzoyl-3-O-benzyl-β-D-glucopyranosyl-(1-4)]-[4,6-benziliden-3-O-fluorenylmethyl-2-deoxy-2phthalimido-β-D-glucopyranosyl-(1-3)]-2,6-di-O-benzoyl-β-D-galactopyranoside 24

Compound 23 (41 mg, 0.08 mmol) and 15a (62 mg, 0.06 mmol) were dissolved in dry DCM (4 mL) with activated molecular sieves and the mixture was stirred for 15 min under nitrogen. NIS (36 mg, 0.16 mmol) and TfOH (1.7 mg, 0.18 mmol) were added at −40° C., and the reaction was stirred overnight at rt, when TLC (7: 3 Tol:EtOAc) showed complete reaction. The reaction was quenched with TEA, molecular sieves were filtered off and the solvent was removed at reduced pressure. The crude was purified by flash chromatography (Tol: EtOAc) to afford 23 (73 mg) in 81% yield.

Trisaccharide 23 (73 mg, 0.05 mmol) was dissolved in dry DCM (4 ml) and 10% of piperidine (0.4 ml) were added at the solution. 10 minutes later, TLC (Tol:Ethyl Acetate) showed complete conversion, and the reaction was concentrated under reduced pressure.

Purification of the crude material by flash chromatography (Tol:EtOAc) gave 24 (57 mg) in 90% yield.

¹H NMR (400 MHz, CDCl₃) δ 7.86-6.63 (m, 34H, H-Ar), 5.55 (s, 1H, CHPha), 5.52 (s, 1H, CHPhb), 5.43 (d, J=8.4 Hz, 1H, H-1^{B}), 5.40 (d, J=7.9 Hz, 1H, H-1^{B}), 5.24 (t, J=7.9 Hz, 1H, H-2^{C}), 4.87 (d, J=12.7 Hz, 1H, CHHPh), 4.73 (d, J=12.7 Hz, 1H, CHHPh), 4.57 (t, J=9.4 Hz, 1H), 4.47 (s, 2H, CH₂Ph), 4.31-4.21 (m, 5H, H-2^{B}, H-4^{A}, H-6^{A}ₐ, CH₂Ph), 4.10 (t, J=8.72 Hz, 1H, H-3^{C}), 4.02 (d, J=7.7 Hz, 1H, H-1^{A}), 3.79 (t, J=9.2 Hz, 1H, H-4^{C}), 3.72-3.51 (m, 10H, H-3^{A}, H-4^{B}, H-5^{B}, H-5^{C}, H-6^{A}_{b}, H-6^{B}_{a,b,} H-6^{C}_{a,b}, OCH₂ₐ), 3.44 (dd, J=2.5, 8.8 Hz, 1H, H-5^{A}), 3.22-3.16 (m, 1H, OCH_{2b}), 2.98-2.86 (m, 3H, H-2^{A}, CH₂N₃), 1.54-1.41 (m, 2H, CH₂CH₂N₃)

¹³C NMR (101 MHz, CDCl₃) δ 170.5, 164.5 (2×CO esters), 134.1-123.4 (C-Ar), 103.3 (C-1^{A}), 102.02 (CHPh), 101.4 (CHPh), 100.4 (C-1^{B}), 99.9 (C-1^{C}), 82.1, 81.8, 78.9, 78.3, 74.2, 73.8, 73.7, 73.6, 72.8, 72.57, 69.0, 68.9, 68.6, 68.3, 66.4, 66.1, 65.7, 57.0, 48.1, 29.0, 26.9.

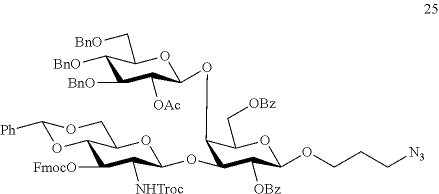

3-Azidopropyl [(2-O-acetyl-3,4,6-tri-O-benzyl-β-D-glucopyranosyl)-(1→4)]-{4,6-O-benzylidene 3-O-(9H-fluoren-9-ylmethyl carbonate)-2-deoxy-2-[[(2,2,2-trichloroethoxy)carbonyl]amino]-β-D-glucopyranosyl-(1→3)}-2,6-di-O-benzoyl-β-D-galactopyranoside 25. A solution of trichloroacetoimidate donor 18 (0.050 g, 0.078 mmol) and acceptor 17a (0.073 g, 0.065 mmol) with 4 Å molecular sieves (0.100 g) in dry DCM (5.0 mL) was stirred for 20 min under nitrogen. TMSOTf (2.4 μL, 0.013) was added at −20° C. After 4 h (TLC; 4:1 Tol: EtOAc) the reaction was quenched with TEA, the solid filtered off and the solvent removed under reduced pressure. The crude was purified by flash chromatography (Tol:EtOAc) to afford trisaccharide 25 in 45% yield (0.127 g).

[α]_{D}²⁵=+16.32° (c 0.25, CHCl₃). ESI HR-MS (C₈₃H₈₁C₁₃N₄O₂₂): m/z=([M+Na]⁺ found 1613.4491; calcd 1613.4306.

¹H NMR (400 MHz, CDCl₃) δ 8.17-7.08 (m, 38H, H-Ar), 5.55 (s, 1H, CHPh), 5.40 (dd, 1H, J=10.1 Hz, J=8.0, H-2^{A}), 5.34 (t, 1H, J=10.1, H-2_{B}), 5.06 (d, 1H, J=8.1 Hz, H-1^{B}), 5.00 (d, 1H, J=8.2 Hz, H-1^{C}), 4.96 (t, 1H, J=8.7 Hz, H-2^{C}), 4.91-4.78 (m, 3H, OCH₂Ph, OCHHPh), 4.74 (dd, 1H, J=12.2 Hz, J=4.2 Hz, OCHHCCl₃), 4.62 (d, 1H, J=10.7 Hz, OCHHPh), 4.59-4.48 (m, 4H, incl. H-1^{A}, OCHHCCl₃, OCH₂Ph), 4.40-4.33 (m, 4H, incl. CH₂^{Fmoc}, H-4^{A}, H-6^{B}ₐ), 4.33-4.26 (m, 1H, H-6^{A}ₐ), 4.26-4.16 (m, 1H, CH^{Fmoc}), 4.01-3.8 (m, 5H, incl. H-3^{C}, H-3^{A}, H-6^{A}_{b}, H-4^{C}, OCHH), 3.8-3.6 (m, 6H, incl. H-6B_{b}, 2H-6^{C}, H-4^{B}, H-5^{A}, OCHH), 3.62-3.40 (m, 4H, incl. H-2^{B}, OCHH, H-5^{C}, H-5^{B}), 3.30-3.08 (m, 2H, CH₂N₃), 2.24 (s, 3H, CH₃), 1.86-1.64 (m, 2H, CH₂CH₂N₃).

¹³C NMR (101 MHz, CDCl₃) δ 170.5, 166.4, 164.9, 154.7, 153.5 (5×CO esters), 143.2-120.0 (C-Ar), 102.1 (C-1^{C}), 101.6 (CHPh), 101.1 (C-1^{A}), 100.0 (C-1^{B}), 82.6 (C-3^{C}), 80.3 (C-3^{A}), 78.7 (C-5^{A}), 78.2 (C-4^{B}), 75.3 (C-5^{B}), 75.2, 74.9 (2×CH₂Ph), 74.2 (C-4^{A}), 74.1 (C-3^{B}), 74.0 (C-4^{A}), 73.7 (C-2^{C}), 73.5 (CH₂Ph), 72.3 (C-4^{C}), 70.8 (C-2^{A}), 70.3)(CH₂^{Fmoc}), 69.2 (C-6^{B}), 68.4 (C-6^{C}), 66.2 (C-5^{C}), 65.6 (OCH₂), 64.5 (CH₂CCl3), 57.5 (C-2^{B}), 48.0 (CH₂N₃), 46.5) (CH^{Fmoc}), 29.0 (CH₂CH₂N₃), 21.2 (CH₃).

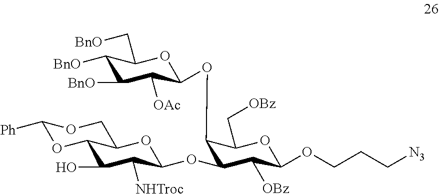

3-Azidopropyl[(2-O-acetyl-3,4,6-tri-O-benzyl-β-D-glucopyranosyl)-(1→4)]-{4,6-O-benzylidene-2-deoxy-2-[[(2,2,2-trichloroethoxy)carbonyl]amino]β-D-glucopyranosyl-(1→3)}-2,6-di-O-benzoyl-β-D-galactopyranoside 26. Trisaccharide 25 was (60.0 mg, 0.038 mmol) was dissolved in 2.0 mL of dry DCM and piperidine (0.2 mL) was added. After 1 h (TLC; 8:2 Tol:EtOAc) the solvent was evaporated under reduced pressure and the crude was purified by flash chromatography (Tol:EtOAc) affording compound 26 (90% yield). [α]_{D}²⁵=−59.72° (c 0.155, CHCl₃).

¹H NMR (400 MHz, CDCl₃) δ 8.12-7.01 (m, 30H, Ar-H), 5.54 (s, 1H, CHPh), 5.35 (dd, 1H; J=10.1 Hz, J=8.0 Hz, H-2^{A}), 5.00-4.86 (m, 3H, incl. H-1^{C}, H-2^{C}, H-1^{B}), 4.85-4.67 (m, 4H, incl. OCH₂Ph, OCHHPh, CHHCCl₃), 4.61-4.43 (m, 5H, incl. H-1^{A}, OCH₂Ph, OCHHPh, CHHCCl₃), 4.43-4.34 (m, 1H, H-6^{A}ₐ), 4.34-4.26 (m, 2H, incl. H-4^{A}, H-6^{B}ₐ), 4.15 (t, 1H, J=8.99 Hz, H-3^{B}), 3.95-3.81 (m, 4H, incl. H-3^{C}, H-3^{A}, OCHH), 3.75-3.60 (m, 5H, incl. H-5^{C}, H-6^{C}_{a,b}, H-6^{B}_{b}, H-6^{A}_{b}), 3.57-3.38 (m, 4H, incl. H-5^{A}, H-4^{B}, H-5^{B}, OCHH), 3.25-3.07 (m, 3H, incl. H-2^{B}, CH₂N₃), 1.80-1.57 (m, 2H, CH₂CH₂N₃).

¹³C NMR (101 MHz, CDCl₃) δ 170.4, 166.4, 165.1 (CO esters), 138.3-126.3 (m, 36C, C-Ar), 101.9 (CHPh), 101.8 (C-1^{B}), 101.1 (C-1^{A}), 100.3 (C1-^{C}), 82.6 (C-3^{C}), 81.4 (C-5^{A}), 80.0 (C-3^{A}), 78.1 (C-5^{C}), 75.3 (C-4^{B}), 75.2, 75.00 (2×CH₂Ph), 74.6 (C-4^{A}), 74.0 (C-2^{C}), 73.8 (OCH₂Ph), 73.5 (C-6^{A}), 72.3 (C-4^{C}), 71.0 (C-2^{A}), 69.7 (C-3^{B}), 69.1 (C-6^{C}), 68.5 (C-6^{B}), 66.1 (C-5^{B}), 65.6 (OCH₂), 64.5 (CH₂CCl₃), 59.4 (C-2^{B}), 47.9 (CH₂N₃), 29.0 (CH₂CH₂N₃), 21.1 (CH₃).

### Synthesis of GBS PSIa Repeating Unit

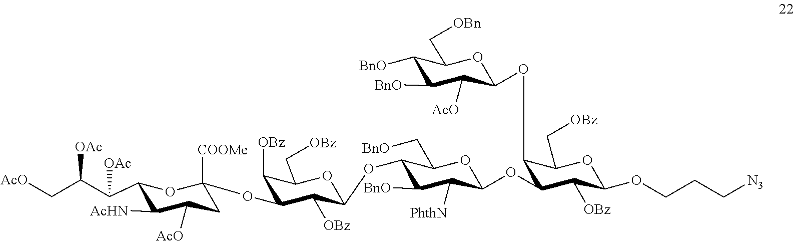

3-Azidopropyl-[(2-O-acetyl-3,4,6-tri-O-benzyl-β-D-glucopyranosyl)-(1→4)]-{2,4,6-tri-O-benzoyl-O-[methyl 4,7,8,9-tetra-O-acetyl-5-N-acetamido-3,5-dideoxy-D-glycero-α-D-galacto-non-2-ulopyranosylonate]-β-D-galactopyranosyl-(1→4)}-3,6-di-O-benzyl-2-deoxy-2-phthalimido-β-D-glucopyranosyl-(1→3)}-2,6-di-O-benzoyl-β-D-galactopyranoside 22. A solution of disaccharide donor 21 (0.124 g, 0.109 mmol) and acceptor 20 (0.110 g, 0.078 mmol) with 4 Å molecular sieves (0.200 g) in dry DCM (5.0 mL) was stirred for 20 min under nitrogen. TMSOTf (2.8 μL, 0.0156 mmol) was added at −20° C. After 4 h (TLC; 6:4 Tol: Acetone) the reaction was quenched with TEA, the solid filtered off and the solvent removed under reduced pressure. The crude was purified by flash chromatography (Tol:Acetone) to afford pentasaccharide 22 in 65% yield (0.120 g).

¹H NMR (400 MHz, CDCl₃) δ 8.39-6.56 (m, 54H, Ar—H), 5.77-5.70 (m, 1H, H-8^{E}), 5.51 (dd, 1H, J=9.8 Hz, J=7.8 Hz, H-2^{D}), 5.34 (d, 1H; J=3.0 Hz, NH), 5.26-5.19 (m, 2H, H-7^{E}, H-1^{D}), 5.16-4.98 (m, 4H, incl. H-1^{B}, H-2^{A}), 4.97-4.72 (m, 7H, incl. H-1^{C}, CHHPh), 4.70-4.42 (m, 7H, CHHPh), 4.42-4.30 (m, 2H, CHHPh), 4.30-4.09 (m, 7H, incl. H-1^{A}, H-6^{E}), 4.08-3.92 (m, 2H), 3.92-3.55 (m, 14, incl. COOCH₃, H-5^{E}, H-2^{B}, OCH₂ₐ), 3.54-3.32 (m, 3H, incl. OCH_{2b}), 3.14-2.88 (m, 2H, CH₂N₃), 2.51-2.41 (dd, 1H, J=12.7 Hz, J=4.3 Hz, H-3^{E}a), 2.13, 2.01, 1.92, 1.87, 1.79 (5×s, 3H each, 5×CH₃CO), 1.73-1.57 (m, 3H, CH₂CH₂N₃, H-3^{E}b), 1.50 (s, 3H, CH₃CO).

### Syntheses of GBS PSIII Structures

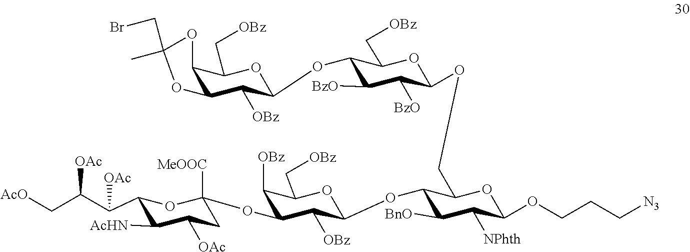

3-Azidopropyl [2,6-di-O-benzoyl-3,4-O-(1-bromomethylethylidene)-β-D-galactopyranosyl-(1-4)-2,3,6-tri-O-benzoyl-β-D-glucopyranosyl-(1-6)]-[2,4,6-tri-O-benzoyl-3-O-(methyl 4,7,8,9-tetra-O-acetyl-5-N-acetamido-3,5-dideoxy-D-glycero-α-D-galacto-non-2-ulopyranosylonate)-β-D-galactopyranosyl-(1-4)]-3-O-benzyl-2-deoxy-2-phthalimido-β-D-glucopyranoside 30. A solution of trisaccharide acceptor 28 (330 mg, 0.24 mmol) and donor 29 (420 mg, 0.60 mmol) with activated molecular sieves (4 A, 800 mg) in DCM (8 mL) was stirred for 20 min under nitrogen. AgOTf (77 mg, 0.30 mmol) was added at −40° C. The reaction mixture was stirred for 10 h at rt, when TLC (7: 3 Tol:acetone) showed complete reaction. TEA was added, the solid filter off and the solvent removed at reduced pressure.

The crude was purified by flash chromatography (Tol:acetone 8:2) to afford 30 (370 mg, 0.16 mmol) in 65% yield. [α]_{D}²⁵=+42.73° (c 1.4, CHCl₃).

¹H NMR (400 MHz, CDCl₃) δ 8.08-6.58 (m, 49H, H-Ar), 5.71 (t, J=8.90 Hz, 1H, H-8^{C}), 5.45-5.37 (m, 2H, H-2^{B}, H-3^{D}), 5.22-5.14 (m, 3H, H-2^{D}, H-4^{B}, H-2^{E}), 5.07 (dd, J=2.84, 9.86 Hz, 1H, H-7^{C}), 4.92 (d, J=10.2, 1H, NH), 4.82-4.68 (m, 5H, H-1^{A}, H-1^{B}, H-3^{D}, H-4^{C}, CHHPh^{A}), 4.42 (d, J=7.2, 1H, H-1^{E}), 4.39-4.24 (m, 5H), 4.20-4.16 (m, 2H, H-1^{D}, CHHPh^{A}), 4.05 (dd, J=5.4, 11.2 Hz, 2H, incl. H-9^{C}), 3.97-3.86 (m, 7H), 3.73 (m, 6H), 3.63-3.42 (m, 7H, incl. OCH₂a), 3.25 (q, J=8.7, 2H, CH₂Br), 3.10-3.05 (m, 1H, OCH_{2b}), 2.91-2.84 (m, 2H), 2.77-2.73 (m, 1H, H-5^{E}), 2.38 (dd, 1H, H-3^{C}ₑ), 2.03, 1.75, 1.71, 1.66, 1,53 (5×s, 3H each, 5×CH₃CO), 1.60 (s, 3H, C(CH₃)), 1.54 (m, 1H, H-3^{C}ₐ).

¹³C NMR (101 MHz, CDCl₃) δ 170.80-164.89 (13×C=O esters) 134.2-122.4 (m, 49, C-Ar), 101.9 (C-1^{D}), 101.0 (C-1^{B}), 100.5 (C-1^{E}), 97.9 (C-1^{A}), 96.9, 80.3, 78.2, 78.0, 75.7, 75.0, 74.6, 74.6, 73.4, 72.7, 72.5, 72.7, 72.6, 72.5, 72.4, 71.1, 70.6, 70.4, 69.5, 68.2, 67.5, 67.0, 66.8, 66.2, 63.2, 62.2, 62.8, 62.3, 61.6, 55.8, 53.2, 48.6, 48.0, 37.3 (C-3^{C}), 37.1, 28.6, 24.6, 23.1, 21.3, 20.8, 20.7, 20.4.

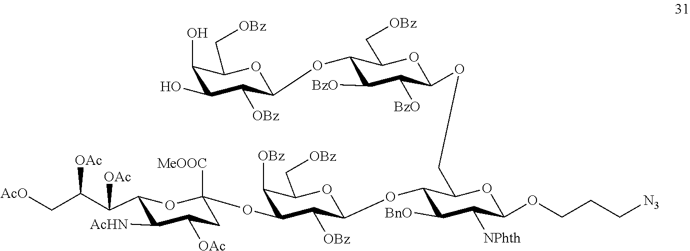

3-Azidopropyl [2,6-di-O-benzoyl-β-D-galactopyranosyl-(1-4)-2,3,6-tri-O-benzoyl-β-Dglucopyranosyl-(1-6)]-[2,4,6-tri-O-benzoyl-3-O-(methyl 4,7,8,9-tetra-O-acetyl-5-N-acetamido-3,5-dideoxy-D-glycero-α-D-galacto-non-2-ulopyranosylonate)-β-D-galactopyranosyl-(1-4)]-3-O-benzyl-2-deoxy-2-phthalimido-β-D-glucopyranoside 31. Pentasaccharide 30 (370 mg, 0.16 mmol) was dissolved in a 90% solution of TFA. After 1 h rt, TLC (Toluene:Acetone 6:4) showed complete reaction. Reaction was concentrated under reduced pressure and purified via flash chromatography (Tol:acetone 7:3) giving 31 (302 mg, 0.13 mmol) in 83% yield as a white solid. [α]_{D}²⁵=+38.78° (c 1.5, CHCl₃).

¹H NMR (400 MHz, CDCl₃) δ 8.08-6.58 (m, 49H, H-Ar), 5.69 (t, J=8.9 Hz, 1H, H-8^{C}), 5.46-5.38 (m, 2H, H-2^{B}, H-3^{B}), 5.26-5.17 (m, 3H, H-2^{B}, H-2^{E}, H-4^{B}), 5.08 (dd, J=2.4, 9.8 Hz, 1H, H-7^{C}), 4.98 (d, J=10.1, 1H, NH), 4.80-4.68 (m, 5H, H-1_{B}, H-1^{A}, H-4^{C}, H-3^{B}, CHHPh), 4.45 (d, J=7.8, 1H, H-1^{E}), 4.39-4.32 (m, 2H, H-9^{C}ₐ, H-6^{E}ₐ), 4.26 (d, J=7.6, 1H), 4.13 (d, J=12.3, 1H, CHHPh), 3.97-3.85 (m, 9H, incl. H-2^{A}, H-4^{E}), 3.72 (m, 6H, incl. H-5^{C}, H-9^{C}_{b}, COOCH₃), 3.64-3.59 (m, 3H), 3.56-3.44 (m, 5H, incl. OCH₂ₐ), 3.16-3.10 (m, 1H, OCH_{2b}), 2.91 (q, J=6.04, 2H), 2.83-2.79 (m, 1H), 2.37 (dd, J=4.62, 12.79, 1H, H-3^{C}), 2.01, 1.83, 1.70, 1.67, 1.52, (5×s, 3H each, 5×CH₃CO), 1.46 (m, 1H, H-3^{C}), 1.36 (m, 2H, CH₂CH₂N₃)

¹³C NMR (101 MHz, CDCl₃) δ 170.0-164.9 (13× C═O esters), 134.0-125.1 (C-Ar), 101.7 (C-1^{B}), 101.0 (C-1^{B}), 101.0 (C-1^{E}), 97.9 (C-1^{A}), 96.9, 80.2, 74.9, 74.6, 73.7, 73.0, 72.5, 72.4, 71.7, 71.6, 71.5, 70.6, 70.1, 69.5, 68.2, 67.0, 66.7, 66.2, 63.1, 62.9, 61.7, 61.5, 55.7, 53.2, 48.6, 48.0, 46.7, 37.3 (C-3^{C}), 28.6, 23.1, 21.3, 20.7, 20.7, 20.4.

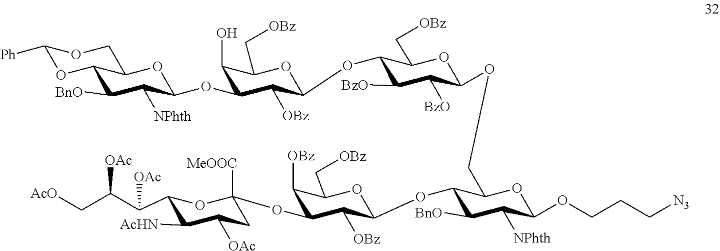

3-Azidopropyl [3-O-benzyl-4,6-O-benzyliden-2-deoxy-2-phthalimido-β-D-glucopyranosyl-(1-3)-2,6-di-O-benzoyl-β-D-galactopyranosyl-(1-4)-2,3,6-tri-O-benzoyl Dglucopyranosyl-(1-6)]-[2,4,6-tri-O-benzoyl-3-O-(methyl 4,7,8,9-tetra-O-acetyl-5-N-acetamido-3,5-dideoxy-D-glycero-α-D-galacto-non-2-ulopyranosylonate)-β-D-galactopyranosyl-(1-4)]-3-O-benzyl-2-deoxy-2-phthalimido-β-D-glucopyranoside 32. A solution of Glucosamine donor 3 (68 mg, 0.11 mmol) and acceptor 31 (190 mg, 0.08 mmol) was stirred for 20 min in dry DCM with activated molecular sieves 4 Å under nitrogen. TfOH was then added at −25° C. and the reaction was stirred for 1 h at 0° C. After that, TLC (Toluene:Acetone 6:4) showed complete reaction, so the mixture was quenched with TEA, the solid was filtered of and the crude was purified with flash chromatography. Hexasaccharide 32 (164 mg, 0.06 mmol) was obtained has a white solid in 69% yield.

[α]_{D}²⁵=+36.31° (c 0.32, CHCl₃).

¹H NMR (400 MHz, CDCl₃) δ 8.04-6.61 (m, 63H, H-Ar), 5.73 (t, J=9.09, 1H, H-8^{C}), 5.55 (s, 1H, CHPh), 5.46 (t, J=8.46, 1H, H-2^{B}), 5.30-5.10 (m, 6H, H-1^{F}, H-3^{D}, H-2^{D}, H-2^{E}, H-4^{B}, H-7^{C}), 4.91 (d, J=10.04, 1H, NH), 4.79-4.68 (m, 6H, H-1^{B}, H-1^{A}, CHHPh, CHHPh), 4.36-4.22 (m, 6H, incl. H-1^{E}), 4.12-4.03 (3H), 3.98-3.87 (m, 7H, incl. H-1^{D}), 3.83-3.67 (m, 11H), 3.62-3.47 (m, 8H, OCH₂ₐ), 3.14-3.08 (m, 1H, OCH_{2b}), 2.94-2.90 (m, 2H, CH₂N₃), 2.49-2.40 (m, 2H, H-3^{C}, H-5^{E}), 2.06, 1.91, 1.77, 1.67, 1.59 (5×s, 3H each, 5×CH₃CO), 1.58 (m, 1H, H-3^{C}), 1.37 (m, 2H, CH₂CH₂N₃).

¹³C NMR (101 MHz, CDCl₃) δ 170.8-164.2 (13×C=O esters), 138.0-125.1 (C-Ar), 102.0 (C-1^{D}), 101.4 (CHPh), 101.0 (C-1^{B}), 100.9 (C-1^{E}), 100.0 (C-1^{F}), 97.9 (C-1^{A}), 82.7, 80.6, 80.53, 78.1, 75.6, 75.0, 74.4, 74.2, 74.0, 72.5, 72.3, 72.1, 71.7, 71.5, 71.4, 71.3, 70.8, 70.6, 70.5, 69.5, 98.5, 68.4, 68.1, 67.1, 66.8, 66.2, 66.1, 63.3, 62.7, 62.5, 61.5, 55.7, 55.5, 53.3, 48.6, 48.0, 37.4 (C-3^{C}), 28.6, 23.1, 21.5, 21.3, 20.8, 20.3.

¹H NMR (400 MHz, D₂O) δ 4.68 (d, J=8.4 Hz, 1H, H.1^{F}), 4.60 (d, J=7.9 Hz, 1H, H-1^{B}), 4.53 (d, J=8.49 Hz, 1H, H-1^{D}), 4.50 (d, J=8.63 Hz, H1, H-1^{A}), 4.43 (d, J=7.77 Hz, 1H, H-1^{E}), 4.30, (d, J=10.5 Hz, 1H, H-6^{A}ₐ), 4.14 (d, J=2.7 Hz, 1H, H-4^{E}), 4.08, (dd, J=2.7, 9.8 Hz, 1H, H-3^{B}), 3.99-3.80 (8H), 3.79-3.54 (22H), 3.46-3.44 (2H), 3.34 (t, J=8.2 Hz, 1H, H-2^{E}), 3.20 (t, J=8.6 Hz, 2H, CH₂N₃), 2.75, (dd, J=4.6, 12.4 Hz, 1H, H-3^{C}_{eq}), 2.03 (s, 6H, 2×CH₃CO), 2.02 (s, 3H, CH₃CO), 1.97 (m, 2H, CH₂CH₂N₃), 1.80 (t, J=12.4 Hz, 1H, H-3^{C}ₐₓ)

¹³C NMR (101 MHz, D₂O) δ 102.91 (C-1^{E}), 102.81 (C-1^{F}), 102.42 (C-1^{D}), 102.12 (C-1^{B}), 101.33 (C-1^{A}), 81.87, 78.20, 77.25, 75.60, 75.00, 74.85, 74.58, 74.24, 73.50, 75.44, 72.91, 72.62, 72.08, 71.75, 69.98, 69.93, 69.33, 68.33, 67.98, 67.51, 67.30, 62.56, 61.05, 60.93, 60.42, 59.99, 55.60, 55.01, 51.63, 49.09, 47.37, 39.59 (C-3^{C}), 23.52, 22.11, 22.01.

### Syntheses of GBS PSIa Fragments

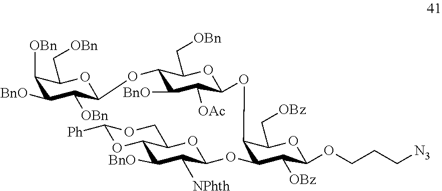

### 3-Azidopropyl 4,6-O-benzilidene-3-O-benzyl-2-deoxy-2-phthalimido-β-D-glucopyranosyl-(1→3)-[(2,3,4,6-tetra-O-benzyl-β-D-galactopyranosyl-(1→4)-2-O-acetyl-3,4,6-tri-O-benzyl-β-D-glucopyranosy-(1→4)]-2,6-di-O-benzoyl-β-D-galactopyranoside 41

A solution of donor 40 (0.900 g, 0.85 mmol) and disaccharide acceptor 9a (0.500 g, 0.53 mmol) with activated 4 Å molecular sieves (0.500 g) in dry DCM (7 mL) was stirred for 20 min under nitrogen. TMSOTf (19 μL, 0.109 mmol) was added at −10° C. After 4 h (TLC; 9:1 DCM: EtOAc) the reaction was quenched with TEA, the solid filtered off and the solvent removed under pressure. The crude was purified by flash chromatography (Tol:EtOAc) to afford the tetrasaccharide 41 in 60% yield (0.590 g) as a white amorphous solid.

¹H NMR (400 MHz, CDCl₃) δ 8.17-6.74 (m, 54H, H-Ar), 5.59 (s, 1H; CHPh), 5.44 (d, 1H, J=8.3 Hz, H-1^{B}), 5.21 (dd, 1H, J=8.3, 10.1, H-2^{C}), 5.18 (d, 1H, J=11.6 Hz, CHHPh), 5.08-5.00 (m, 3H, incl. H-1^{C}, H-2^{C}), 4.95 (d, 2H, J=2.5 Hz, CH₂Ph), 4.82-4.74 (m, 4H), 4.70 (dd, 1H, J=3.3, 12.0 Hz, H-6^{A}ₐ), 4.65-4.31 (m, 10H, incl. H-6^{A}_{b}, H-1^{D}, H-1^{A}), 4.30-4.22 (m, 2H, incl. H-2^{B}), 4.07-3.72 (m, 11H, incl. H-3^{A}, H-3^{C}, OCHH), 3.70-3.61 (m, 1H), 3.58-3.49 (m, 3H), 3.48-3.34 (m, 4H, incl. OCHH), 3.14-2.95 (m, 2H, CH₂N₃), 1.93 (s, 3H, COCH₃), 1.74-1.53 (m, 2H, CH₂CH₂N₃)

¹³C NMR (101 MHz, CDCl₃) δ 170.3, 167.6, 166.9, 166.4, 164.5 (5×CO), 139.6-122.9 (m, 68^{C}, C-Ar), 103.1 (C-1^{B}), 101.4 (CHPh), 100.9 (C-1^{A}), 100.3 (C-1^{C}), 99.8 (C-1^{B}), 83.1, 82.6, 81.4, 80.1, 79.2, 75.8, 75.3, 74.7, 74.6, 74.5, 74.3, 73.8, 73.7, 73.5, 73.2, 73.1, 72.6 (C-2^{C}), 72.6, 72.3, 70.7 (C-2^{A}), 68.7, 68.5, 68.2 (OCH²), 66.1, 65.0, 64.6 (C-6^{A}), 55.9 (C-2^{B}), 47.9 (CH₂N₃), 28.9 (CH₂CH₂N₃), 20.5 (CH₃).

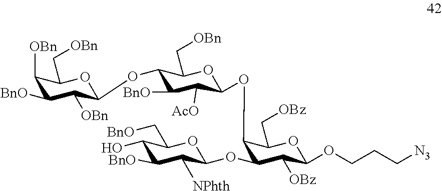

3-Azidopropyl 3,6-di-O-benzyl-2-deoxy-2-phthalimido-β-D-glucopyranosyl-(1→3)-[(2,3,4,6-tetra-O-benzyl-β-D-galactopyranosyl-(1→4)-2-O-acetyl-3,4,6-tri-O-benzyl-β-D-glucopyranosy-(1→4)]-2,6-di-O-benzoyl-β-D-galactopyranoside 42.

A solution of 41 (0.380 g, 0.21 mmol) in AcCN (10 mL) was cooled at 0° C. Me₃NBH₃ (0.075 g, 1.03 mmol) and BF₃·OEt₂ (0.127 mL, 1.03 mmol) were added and the reaction was stirred for 2 h under nitrogen. TLC showed complete reaction (8:2 toluene:EtOAc). First TEA and then MeOH were added until neutral pH. The solvent removed at reduced pressure and the crude was purified by flash chromatography (toluene:EtOAc) to afford 42 in 52% yield (0.200 g).

¹H NMR (400 MHz, CDCl₃) δ 8.11-6.77 (m, 54H, H-Ar), 5.31 (d, 1H, J=7.7 Hz, H-1^{B}), 5.18-5.08 (m, 2H, incl. H-2^{A}), 5.04-4.84 (m, 6H, incl. H-1^{C}, H-2^{C}, 3×CH₂Ph), 4.78-4.26 (m, 16H, incl. H-1D, H-6^{A}ₐ, H-6^{A}_{b}), 4.26-4.05 (m, 3H), 3.99 (t, 1H, J=9.4 Hz), 3.95-3.89 (m, 2H), 3.89-3.69 (m, 7H), 3.69-3.54 (m, 2H), 3.54-3.27 (m, 6H), 3.10-2.91 (m, 2H), 1.77 (s, 3H), 1.66-1.49 (m, 2H).

¹³C NMR (101 MHz, CDCl₃) δ 170.3, 166.3, 164.6 (3×CO), 139.7-126.8 (m, 68 C, C-Ar), 102.9 (C-1^{D}), 100.9 (C-1^{A}), 100.3 (C-1^{C}), 99.1 (C-1^{B}), 82.6, 81.3, 80.0, 78.7, 78.6, 75.5, 75.1, 74.7, 74.6, 74.5, 74.3, 74.0, 73.7, 73.6, 73.6, 73.4, 73.2, 73.0, 72.6, 72.5, 72.4 (C-2^{C}), 70.8, 70.6 (C-2^{A}), 68.5, 68.2 (OCH₂), 64.9, 64.8, 55.6 (C-2^{B}), 48.0 (CH₂N₃), 28.9 (CH₂CH₂N₃), 20.4 (CH₃).

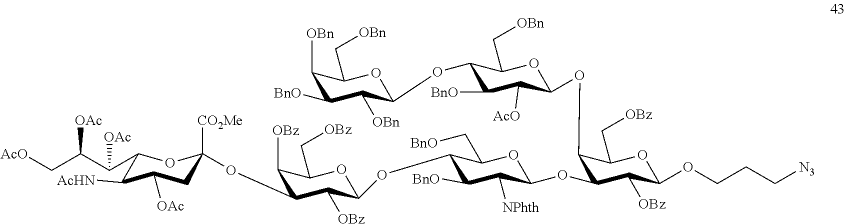

### 3-Azidopropyl 3-O-(5-acetamido-3,5-dideoxy-D-glycero-α-D-galacto-non-2-ulopyranosyl)-2,4,6-tri-O-benzoyl-β-D-galactopyranosyl-(1→4)-3,6-di-O-benzyl-2-deoxy-2-phthalimido-β-D-glucopyranosyl-(1→3)-[2,3,4,6-tetra-O-benzyl-β-D-galactopyranosyl-(1→4)-2-O-acetyl-3,4,6-tri-O-benzyl-β-D-glucopyranosyl)-(1→4)]-2,6-di-O-benzoyl-β-D-galactopyranoside 43

A solution of donor 21 (0.162 g, 0.143 mmol) and tetrasaccharide acceptor 42 (0.176 g, 0.095 mmol) with activated 4 Å molecular sieves (0.200 g) in dry DCM (3 mL) was stirred for 20 min under nitrogen. TMSOTf (3.4 μL, 0.019 mmol) was added at −10° C. After 4 h (TLC; 6:4 Toluene: acetone) the reaction was quenched with TEA, the solid filtered off and the solvent removed under pressure. The crude was purified by flash chromatography (Tol:acetone) to afford the tetrasaccharide 43 in 78% yield (0.206 g) as a white amorphous solid.

¹H NMR (400 MHz, CDCl₃) δ 8.36-6.54 (m, 69H, H-Ar), 5.74-5.66 (m, 1H, H-8^{F}), 5.48 (dd, 1H, J=7.4, 9.6 Hz, H-2^{E}), 5.33 (d, 1H, J=3.2 Hz, H-4^{E}), 5.22 (dd, 1H, J=2.3, 9.6 Hz, H-7^{F}), 5.18-5-04 (m, 4H, incl. H-2^{A}, H-1^{E}), 4.99-4.90 (m, 3H, incl. H-3^{E}, H-1^{B}), 4.90-4.77 (m, 4H), 4.75 (s, 2H, OCH₂Ph), 4.72-4.63 (m, 3H), 4.61-4.38 (m, 8H, incl. H-1^{A}), 4.35-4.08 (m, 10H), 4.09-3.93 (m, 4H, incl. H-9^{F}), 3.92-3.64 (m, 12H, COOCH₃), 3.64-3.31 (m, 8H, incl. OCHH), 3.30-3.24 (m, 1H, OCHH), 3.10-2.89 (m, 2H, CH₂N₃), 2.43 (dd, 1H, J=4.5, 12.4 Hz, H-3^{F}_{eq}), 2.14 (s, 3H, COCH₃), 1.98 (s, 3H, COCH₃), 1.91 (s, 3H, COCH₃), 1.78 (s, 3H, COCH₃), 1.70-1.57 (m, 7H, incl. CH₂CH₂N₃, H-3^{F}ₐₓ, COCH₃), 1.50 (S, 3H, COCH₃).

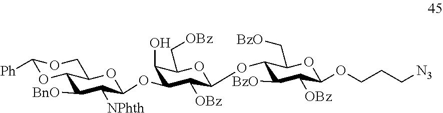

### 3-Azidopropyl 4,6-O-benzilidene-3-O-benzyl-2-deoxy-2-phthalimido-β-D-glucopyranosyl-(1→3)-2,6-di-O-benzoyl-β-D-galactopyranosyl-(1→4)-2,3,6-tri-O-benzoyl-β-D-glucopyranoside 45

A solution of donor 3 (0.450 g, 0.71 mmol) and disaccharide acceptor 44 (0.518 g, 0.548 mmol) with activated 4 Å molecular sieves (0.500 g) in dry DCM (7 mL) was stirred for 20 min under nitrogen. TMSOTf (19 μL, 0.109 mmol) was added at −10° C. After 4 h (TLC; 9:1 DCM: EtOAc) the reaction was quenched with TEA, the solid filtered off and the solvent removed under pressure. The crude was purified by flash chromatography (DCM:EtOAc) to afford the thrisaccharide 45 in 60% yield (0.465 g) as a white amorphous solid.

¹H NMR (400 MHz, CDCl₃) δ 8.14-6.72 (m, 39H, H-Ar), 5.62 (t, 1H, J=9.5 Hz, H-3^{A}), 5.59 (s, 1H, CHPh), 5.35 (dd, 1H, J=7.9, 9.6 Hz, H-2^{A}), 5.31 (d, 1H, J=7.5 Hz, H-1^{C}), 5.26 (dd, 1H, J=8.1, 9.5 Hz, H-2^{B}), 4.70 (d, 1H, J=12.4 Hz, OCHHPh), 4.55 (d, 1H, J=7.7 Hz, H-1^{A}), 4.45 (d, 1H, J=8.0 Hz, H-1^{B}), 4.39 (d, 1H, J=12.4 Hz, OCHHPh), 4.42-4.10 (m, 6H, incl. H-6^{A}, H-6^{B}a, H-3^{C}, H-2^{C}), 4.05 (t, 1H, J=9.5 Hz, H-4^{A}), 3.97 (d, 1H, J=3.0 Hz, H-4^{B}), 3.85-3.72 (m, 3H, incl. H-5^{A}, H-6^{C}b, OCHH), 3.69 (dd, 1H, J=3.3, 9.8 Hz, H-3^{B}), 3.66-3.55 (m, 3H, incl. H-4^{C}, H-6^{B}b, H-5^{C}), 3.55-3.49 (m, 1H, H-5^{B}), 3.49-3.41 (m, 1H, OCHH), 3.21-3.09 (m, 2H, CH₂N₃), 1.80-1.58 (m, 2H, CH₂CH₂N₃)

¹³C NMR (101 MHz, CDCl₃) δ 166.0, 165.8, 165.5, 165.2, 164.0 (5×CO esters), 137.6-126.1 (m, C-Ar), 101.3 (CHPh), 101.0 (C-1^{A}), 100.6 (C-1^{B}), 99.8 (C1-^{C}), 82.6, 80.6, 75.3, 74.2, 74.0, 72.9, 72.5, 72.1, 71.7, 70.6, 68.5, 68.3, 66.5, 66.1, 62.6, 62.3, 55.5 (C-2^{C}), 47.8 (CH₂N₃), 28.9 (CH₂CH₂N₃).

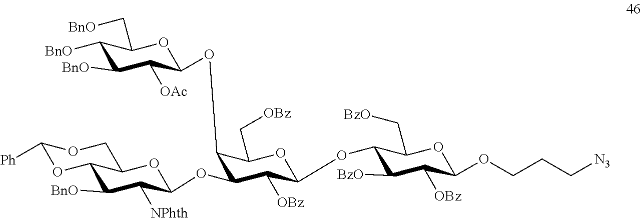

### 3-Azidopropyl 4,6-O-benzilidene-3-O-benzyl-2-deoxy-2-phthalimido-β-D-glucopyranosyl-(1→3)-[(2-O-acetyl-3,4,6-tri-O-benzyl-β-D-glucopyranosyl-(1→4)]-2,6-di-O-benzoyl-β-D-galactopyranosyl-(1→4)-2,3,6-tri-O-benzoyl-β-D-glucopyranoside 46

A solution of donor 47 (0.303 g, 0.476 mmol) and trisaccharide acceptor 46 (0.450 g, 0.317 mmol) with activated 4 Å molecular sieves (0.400 g) in dry DCM (7 mL) was stirred for 20 min under nitrogen. TMSOTf (11 μL, 0.063 mmol) was added at −10° C. After 4 h (TLC; 9:1 DCM: EtOAc) the reaction was quenched with TEA, the solid filtered off and the solvent removed under pressure. The crude was purified by flash chromatography (Tol:EtOAc) to afford the thrisaccharide x in 65% yield (0.380 g) as a white amorphous solid.

¹H NMR (400 MHz, CDCl₃) δ 8.17-6.73 (m, 54H, H-Ar), 5.66-5.57 (m, 2H, incl CHPh, H-3^{A}), 5.38 (dd, 1H, J=7.9, 9.6 Hz, H-2^{A}), 5.33 (d, 1H, J=8.2 Hz, H-1^{C}), 5.08 (dd, 1H, J=7.9, 10.0, H-2^{B}), 4.96-4.83 (m, 5H, incl. H-1^{D}, H-2^{D}, 3×CH₂OBn), 4.75-4.63 (m, 3H, incl. 3×CH₂OBn), 4.55 (d, 1H, J=7.7 Hz H-1^{A}), 4.51 (d, 1H, J=12.0 Hz, 1×CH₂OBn), 4.44, 4.04 (m, 8H, incl. 1×CH₂OBn, H-4^{B}, H-2^{C}, H-3^{C}), 4.01-3.87 (m, 2H, H-4^{A}, H-3^{D}), 3.85-3.65 (m, 7H, incl. OCH₂′, H-5^{B}, H-5^{A}, H-3^{B}, 2×CH₂OBn), 3.64-3.51 (m, 3H), 3.51-3.38 (m, 3H, incl. OCH₂″), 3.23-3.07 (m, 3H, incl. CH₂N₃), 2.09 (s, 3H, COCH₃), 1.81-1.58 (m, 2H, CH₂CH₂N₃).

¹³C NMR (101 MHz, CDCl₃) δ 170.4, 167.5, 166.8, 165.93, 165.8, 165.3, 164.0, 163.4 (8×CO), 138.6-122.8 (m, C-Ar), 101.3 (CHPh), 101.1 (C-1^{A}), 100.6 (C-1^{B}), 100.1 (s, 2^{C}, C-1^{C}, C-1^{D}), 83.4 (C-3^{D}), 82.9 (C-5^{B}), 79.7, 77.9, 75.6 (C-4^{A}), 75.4, 75.3, 75.1, 74.3, 74.2 (C-4^{B}), 73.5 (C-3^{C}), 73.4, 73.1 (C-2D), 72.9, 72.4 (C-3^{A}), 72.3, 71.5 (C-2^{A}), 70.9 (C-3^{B}), 69.1, 68.5, 66.6 (OCH₂), 66.0, 63.1, 62.4, 55.8 (C-2^{C}), 47.9 (CH₂N₃), 28.9 (CH₂CH₂N₃), 20.6 (CH₃).

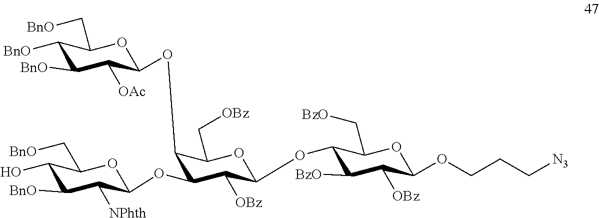

### 3-Azidopropyl 3,6-di-O-benzyl-2-deoxy-2-phthalimido-β-D-glucopyranosyl-(1→3)-[(2-O-acetyl-3,4,6-tri-O-benzyl-β-D-glucopyranosyl)-(1→4)]-2,6-di-O-benzoyl-β-D-galactopyranosyl-(1→4)-2,3,6-tri-O-benzoyl-β-D-glucopyranoside 47

A solution of 46 (0.370 g, 0.195 mmol) in AcCN (10 mL) was cooled at 0° C. Me₃NBH₃ (0.071 g, 0.98 mmol) and BF₃·OEt₂ (0.120 mL, 0.98 mmol) were added and the reaction was stirred for 2 h under nitrogen. TLC showed complete reaction (8:2 toluene:EtOAc). First TEA and then MeOH were added until neutral pH. The solvent removed at reduced pressure and the crude was purified by flash chromatography (toluene:EtOAc) to afford 47 in 55% yield (0.200 g).

¹H NMR (400 MHz, CDCl₃) δ 8.36-6.63 (m, 54H Ar—H), 5.59 (t, 1H, J=9.4 Hz, H-3^{A}), 5.37 (t, 1H, J=8.2 Hz, H-2^{A}), 5.24 (d, 1H, 7.3 Hz, H-1^{C}), 5.05 (dd, 1H, J=8.6, 9.8 Hz, H-2^{B}), 4.93-4.80 (m, 5H, incl. H-1^{D}, H-2^{D}, 3×OCH₂Ph), 4.70-4.58 (m, 2H, 2×OCH₂Ph), 4.58-4.23 (m, 8H, incl. H-1^{A}, H-1^{B}, H-6^{A}ₐ, 5×CH₂OPh), 4.23-4.00 (m, 6H, incl. H-6^{A}_{b}, H-2^{C}), 3.95 (t, 1H, J=9.1 Hz, H-4^{A}), 3.91-3.82 (m, 1H, H-3^{D}), 3.83-3.32 (m, 14H, incl. OCH₂, H-4^{D}, H-5^{A}, H-3^{B}), 3.23-3.01 (m, 3H, incl. CH₂N₃), 2.00 (s, 3H·COCH₃), 1.80-1.55 (m, 2H, CH₂CH₂N₃).

¹³C NMR (101 MHz, CDCl₃) δ 170.2, 165.9, 165.8, 165.5, 165.3, 164.0 (6×CO), 138.7-122.9 (m, C-Ar), 101.1 (C-1^{A}), 100.6 (C-1^{B}), 100.1 (C-1^{D}), 99.5 (C-1^{C}), 83.4 (C-3^{D}), 79.4, 78.4, 77.8, 75.5 (C-4^{A}), 75.4, 75.2, 75.0, 74.2, 73.6, 73.5, 73.4, 73.0 (C-2^{D}), 72.4 (C-3^{A}), 71.5 (C-2^{A}), 71.0 (C-2^{B}), 70.6, 69.1, 66.6 (OCH²), 63.4, 62.5 (C-6^{A}), 55.4 (C-2^{C}), 47.9 (CH₂N₃), 28.9 (CH₂CH₂N₃), 20.5 (CH₃).

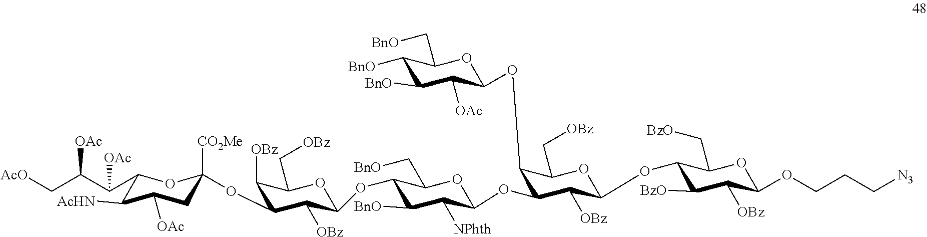

### 3-Azidopropyl 3-O-(5-acetamido-3,5-dideoxy-D-glycero-α-D-galacto-non-2-ulopyranosyl)-2,4,6-tri-O-benzoyl-β-D-galactopyranosyl-(1→4)-3,6-di-O-benzyl-2-deoxy-2-N-phthalimido-β-D-glucopyranosyl-(1→3)-[O-acetyl-3,4,6-tri-O-benzyl-β-D-glucopyranosyl-(1→4)]-2,6-di-O-benzoyl-β-D-galactopyranosyl}-(1→4)-2,3,6-tri-O-benzoyl-β-D-glucopyranoside 48

A solution of donor x (0.900 g, 0.85 mmol) and disaccharide acceptor 9a (0.500 g, 0.53 mmol) with activated 4 Å molecular sieves (0.500 g) in dry DCM (7 mL) was stirred for 20 min under nitrogen. TMSOTf (19 μL, 0.109 mmol) was added at −10° C. After 4 h (TLC; 9:1 DCM: EtOAc) the reaction was quenched with TEA, the solid filtered off and the solvent removed under pressure. The crude was purified by flash chromatography (Tol:EtOAc) to afford the tetrasaccharide 48 in 60% yield (0.590 g) as a white amorphous solid.

¹H NMR (400 MHz, CDCl₃) δ 8.28-6.55 (m, 69H, H-Ar), 5.71-5.63 (m, 1H, H-8^{F}), 5.54 (t, 1H, J=9.0 Hz, H-3^{A}), 5.46 (dd, 1H, J=8.0, 10.0 Hz, H-2^{E}), 5.34 (t, 1H, J=8.5 Hz, H-2^{A}), 5.29 (d, 1H, J=3.3 Hz, H-4^{E}), 5.20 (dd, 1H, J=2.2, 9.7, H-7^{F}), 5.07 (dd, 2H, J=5.9, 7.8 Hz, H-1^{E}, H-1^{D}), 5.02-4.73 (m, 9H, incl. H-2^{B}, H-3^{E}, H-1^{C}, H-4^{F}, H-5^{E}), 4.60 (d, 1H, J=10.9 Hz, OCHHPh), 4.55-4.36 (m, 5H, incl. H-1^{A}), 4.33-4.23 (m, 4H, incl. H-1^{B}), 4.23-3.88 (m, 10H, incl. H-9^{F}, OCHH), 3.88-3.61 (m, 10H, incl. H-2^{C}, COOCH₃), 3.61-3.53 (m, 3H), 3.50 (bd, 1H, J=7.8 Hz, H-3^{C}), 3.46-3.34 (m, 3H), 3.33-3.25 (m, 2H), 3.19-3.08 (m, 2H, CH₂N₃); 3.01 (dd, 1H, J=7.7, 11.6 Hz, OCHH), 2.41 (dd, 1H, J=4.4, 12.4, H-3^{F}_{eq}), 2.09 (s, 3H, CH₃), 1.98 (s, 3H, CH₃), 1.93 (s, 3H, CH₃), 1.90 (s, 3H, CH₃), 1.78 (s, 3H, CH₃), 1.65-1.54 (m, 4H, H-3^{F}ₐₓ, CH₂CH₂N₃), 1.47 (s, 3H, CH₃).

### General Procedure for Deprotection

A mixture of protected oligosaccharide (0.1 mmol) and LiI (3 mmol) in pyridine (5 mL) was heated for 24 h at 120° C. The reaction mixture was concentrated under vacuum, and the residue was purified by silica gel column chromatography (gradient 2% MeOH in DCM) to afford the demethylated product. This material was dissolved in ethanol (4 mL), and ethylenediamine (400 μL) was added. After being stirred for 16 h at 90° C., the reaction mixture was then concentrated in vacuo, and the residue was coevaporated from Toluene (2×10 mL) and EtOH (2×5 mL). The crude mixture was re-dissolved in pyridine (5 mL), and acetic anhydride (5 mL) was added. After being stirred for 16 h at room temperature, the reaction mixture was concentrated under reduced pressure and the residue was purified by silica gel column chromatography (gradient 10% MeOH in DCM). The residue was dissolved in MeOH and MeONa was added until pH=13.

After 48 h the reaction was neutralized and the solvent removed under vacuum and the crude was purified with C18 5 g column (gradient 20% MeOH in H₂O). The residue was finally dissolved in MeOH and Pd/C (1: 1 w/w in respect to the sugar) was added. The reaction mixture was stirred under pressure of H₂ (3 bar) for 72 h. Then, the catalyst was filtered off and the filtrate concentrated under reduced pressure. The reaction mixture was purified by G-10 size-exclusion column chromatography using water for elution.

Fractions containing the sugar were quantified by sialic acid assay and freeze-dried to afford the deprotected oligosaccharide as an amorphous powder (31% yield for 32a, 45% for 36, 30% for 37).

Compound 32a: [α]_{D}²⁵=+1.56° (c 0.81, H₂O). ESI MS m/z [M+Na]⁺ found 1281,4932; calcd 1281,4610.

¹H NMR (400 MHz, D₂O) δ 4.68 (d, J=8.4 Hz, 1H, H-1^{F}), 4.60 (d, J=7.9 Hz, 1H, H-1^{B}), 4.53 (d, J=8.49 Hz, 1H, H-1^{B}), 4.50 (d, J=8.63 Hz, H1, H-1^{A}), 4.43 (d, J=7.77 Hz, 1H, H-1^{E}), 4.30, (d, J=10.5 Hz, 1H, H-6^{A}ₐ), 4.14 (d, J=2.7 Hz, 1H, H-4^{E}), 4.08, (dd, J=2.7, 9.8 Hz, 1H, H-3^{B}), 3.99-3.80 (8H), 3.79-3.54 (22H), 3.46-3.44 (2H), 3.34 (t, J=8.2 Hz, 1H, H-2^{E}), 3.20 (t, J=8.6 Hz, 2H, CH₂N₃), 2.75, (dd, J=4.6, 12.4 Hz, 1H, H-3^{C}_{eq}), 2.03 (s, 6H, 2×CH₃CO), 2.02 (s, 3H, CH₃CO), 1.97 (m, 2H, CH₂CH₂N₃), 1.80 (t, J=12.4 Hz, 1H, H-3^{C}ₐₓ). (FIG. 1)

Compound 36: ¹H NMR (400 MHz, D₂O) δ 4.56 (d, 1H, J=8.4 Hz), 4.42 (d, 1H, 7.9 Hz), 4.39-4.34 (m, 1H), 4.30 (d, 1H, J=7.8 Hz), 4.02 (d, 1H, J=3.3 Hz), 3.98 (dd, 1H, J=3.1, 9.9 Hz), 3.95-3.39 (m, 28H), 3.23-3.13 (m, 2H), 3.06-2.98 (m, 1H), 2.62 (dd, 1H, J=4.7, 12.6 Hz, H-3D_{eq}), 1.96-1.82 (m, 8H, incl. 2×CH₃), 1.66 (t, 1H, J=12.2 Hz, H-3^{D}ₐₓ). (FIG. 3) Identical as reported in the literature (Cattaneo, V et al. Synthesis of Group B Streptococcus type III polysaccharide fragments for evaluation of their interactions with monoclonal antibodies. Pure and Applied Chemistry 2017, 89(7), 855-875).

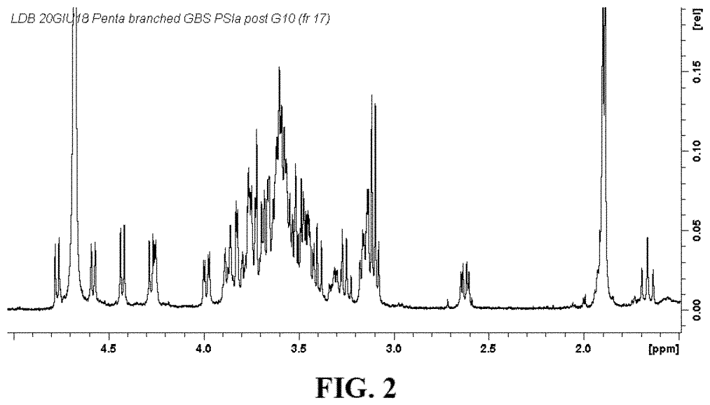

Compound 37: ¹H NMR (400 MHz, D₂O) δ 4.77 (d, 1H, J=7.7 Hz), 4.58 (d, 1H, 8.4 Hz), 4.42 (d, 1H, J=8.4 Hz), 4.31-4.22 (m, 2H), 3.98 (dd, 1H, J=2.9, 9.7 Hz), 3.91-3.36 (m, 30H), 3.36-3.20 (m, 2H), 3.20-3.06 (m, 2H), 2.62 (dd, 1H, J=4.6, 12.3 Hz, H-3^{D}_{eq}), 1.90 (s, 3H, COCH₃), 1.88 (s, 3H, COCH₃), 1.96-1.86 (m, 2H), 1.66 (t, 1H, J=12.3 Hz, H-3^{D}ₐₓ). (FIG. 2)

### Typical Protocol for Conjugation

A solution of di-N-hydroxysuccinimidyl adipate (10 eq) and triethylamine (0.2 eq) in DMSO was added to amine oligosaccharides. The reaction was stirred for 3 h, then the product was precipitate at 0° C. by adding ethyl acetate (9 volumes). The solid was washed 10 times with ethyl acetate (2 volumes each) and lyophilized. The activated sugar was incubated overnight with CRM₁₉₇ in sodium phosphate 100 mM at a protein concentration of 5-10 mg/ml, using 50-100 mol saccharide/mol protein ratio.

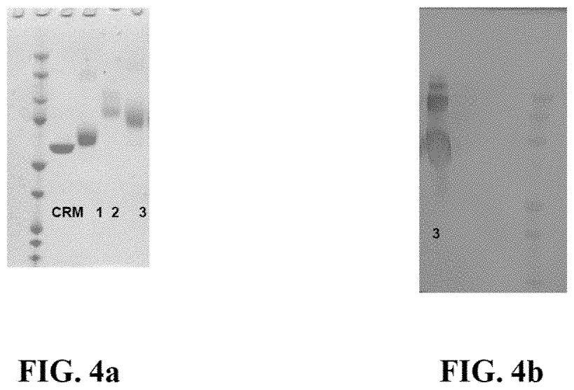

SDS-Page and Western immunoblotting analysis (FIG. 4). Sodium Dodecyl Sulfate-Polyacrilamide gel electrophoresis (SDS-Page) was performed on 4-12% pre-casted polyacrylamide gel (NuPAGE®Invitrogen) using MOPS 1× as running buffer (NuPAGE®Invitrogen). 5 μg of protein were loaded for each sample. After electrophoretic running with a voltage of 150V for about 45 minutes, the gel was stained with blue coomassie.

For western blot, the protein bands of the SDS-page were transferred onto a nitrocellulose membrane in an iBlot® 7-Minute Blotting System (Invitrogen). The membrane was blocked for 1 h at room temperature with 2% BSA in PBS-T (blocking buffer), then it was incubated for 2 h with a 1:1000 dilution of anti PSIII serum (from mice immunized with PSIII conjugated to a GBS pilus protein) in the same buffer. The membrane was washed 3 times with H₂O and incubated with peroxidase-labeled goat anti-mouse (Sigma-Aldrich) in blocking buffer at room temperature for 1 h. After washing in PBS-T, PBS and H₂O, the membrane was dipped in the color development solution (BIO RAD) for 10 min at room temperature and finally washed with H₂O.

FIG. 4. Characterization of glycoconjugate 32a-CRM₁₉₇. (A) SDS Page electrophoresis and (B) Western blot with anti GBS PSIII murine serum. (1. branched-CRM₁₉₇ ˜2.5 mol/mol ratio; 2. branched-CRM₁₉₇˜20 mol/mol ratio; 3.32a-CRM197)

TABLE 4
Characteristics of the synthesized glycoconjugate 32a-CRM₁₉₇

| | Saccharide | Protein | Sacch/Prot | Structure |
|---|---|---|---|---|
| Sample | (Gal) μg/mL | μg/mL | (mol/mol) | MW |
| GBS PSIII Hexa- | 44.9 | 270.0 | 7.8 | 1250 g/mol |
| CRM lot. | | | | |
| LDB04Apr18 | | | | |

1. A compound of formula: 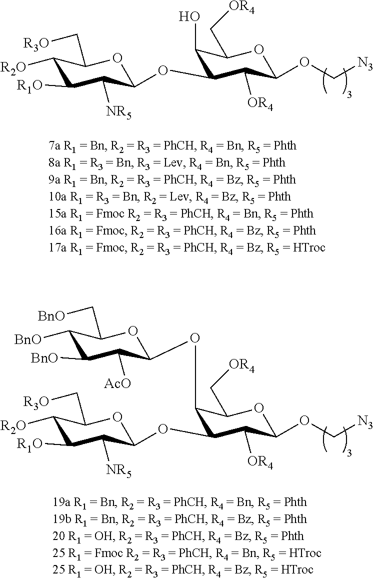
or a salt thereof.

2. A compound according to claim 1 of formula: 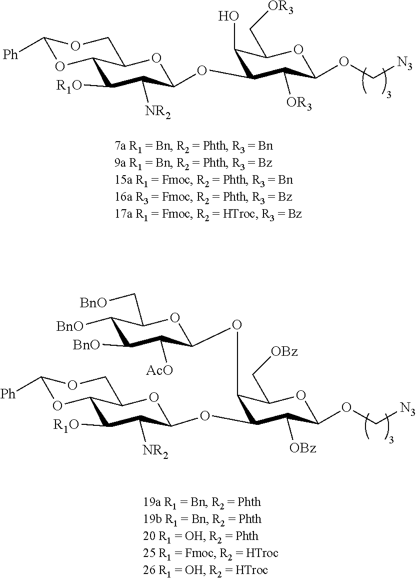
or a salt thereof.

3. The compound according to claim 2 selected from 7a and 9a.

4. The compound according to claim 2 selected from 19a and 19b.

5. The compound according to claim 2 selected from 16a and 17a.

6. The compound according to claim 2 selected from 25 and 26.

7. Use of the compound 7a, or 9a as intermediate for the preparation of compounds of formula 19a and 19b.

8. Use of the compound 16a or 17a as intermediate for the preparation of compounds 25 or 26.

9. Use of compound 19a or 19b as intermediate for the preparation of the repeating unit of the GBS PS Ia

10. Use of compound 25 or 26 as intermediate for the preparation of the repeating unit of the GBS PS Ib or Ia.

11. Process for the preparation of the repeating unit of the GBS PS Ia or Ib, comprising reacting the compound of formula 25 or 26 with compound 21, in the presence of Me₃NBH₃/BF₃Et₂O, or TMSOTf/DCM, according to the following scheme:

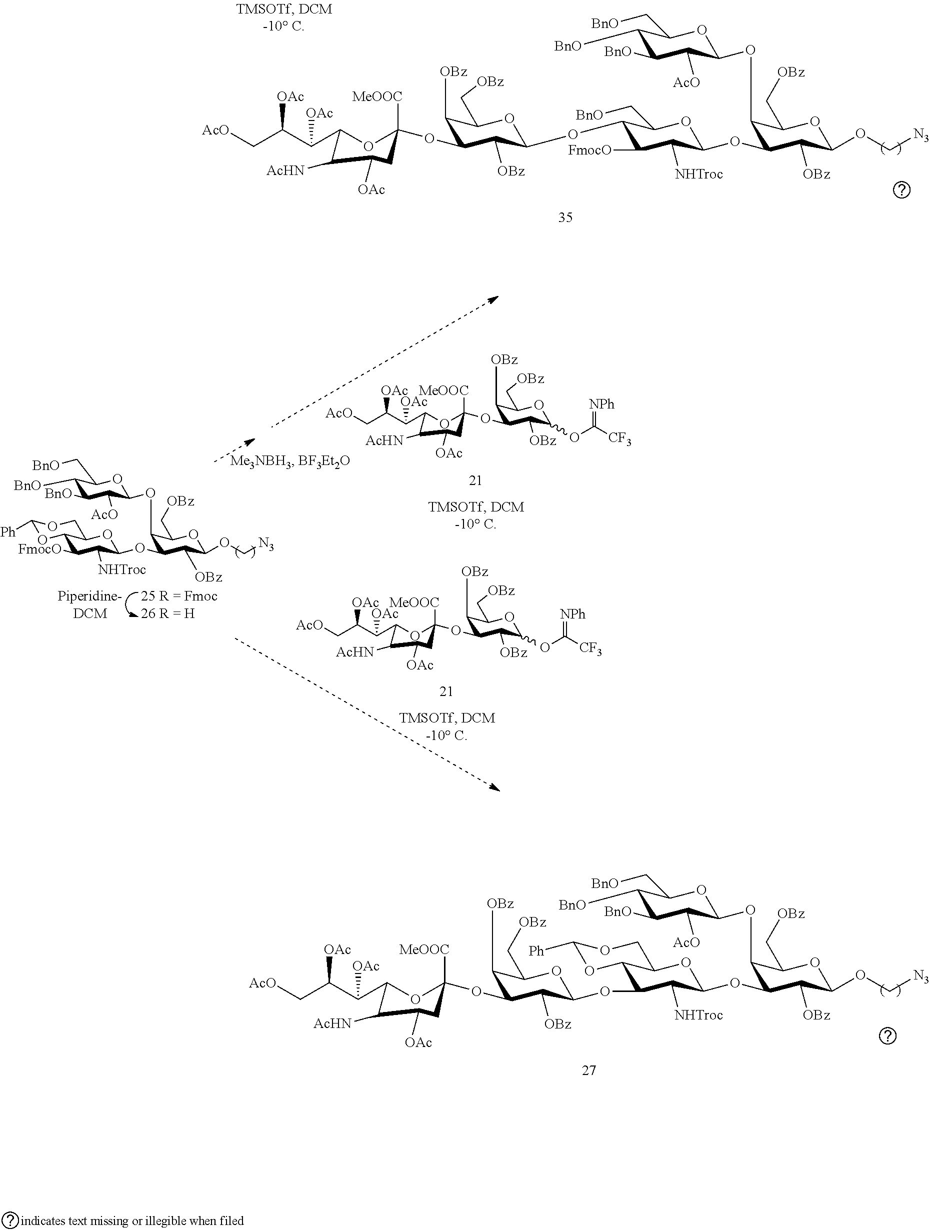

12. The compounds of claims 1-6, conjugated to a carrier protein.

13. The compounds of claim 12, wherein said carrier protein is selected from the group consisting of: CRM197, tetanus toxoid (TT), tetanus toxoid fragment C, protein D, non-toxic mutants of tetanus toxin and diphtheria toxoid (DT).

14. The compounds of claim 13, wherein said carrier protein is CRM197.
